(19) United States
(12) Patent Application Publication
(10) Pub. No.: US 2023/0346633 A1
(43) Pub. Date: Nov. 2, 2023

(54) VIBRATING CAPSULE SYSTEMS AND TREATMENT METHODS USING SAME

(71) Applicant: Vibrant Ltd., Yokneam (IL)

(72) Inventors: Lior BEN-TSUR, Netanya (IL); Shai MOLNAR, Shorashim (IL); Ronny SHABBAT, Kibbutz Yizra'el (IL)

(21) Appl. No.: 18/141,892

(22) Filed: May 1, 2023

Related U.S. Application Data
(63) Continuation-in-part of application No. 17/044,258, filed on Oct. 1, 2020.
Application No. 17/044,258 is a continuation-in-part of application No. 16/185,044, filed on Nov. 9, 2018, now Pat. No. 10,543,348.
Application No. 16/185,044 is a continuation-in-part of application No. 16/178,425, filed on Nov. 1, 2018, now Pat. No. 10,537,720.
Application No. 16/178,425 is a continuation-in-part of application No. 16/377,213, filed on Apr. 7, 2019, now Pat. No. 11,638,678.
Application No. 16/377,213 is a continuation-in-part of application No. 17/419,187, filed on Jun. 30, 2021.
Application No. 17/419,187 is a continuation-in-part of application No. 17/246,643, filed on May 2, 2021.
Application No. 17/246,643 is a continuation-in-part of application No. PCT/IB2020/050433, filed on Jan. 21, 2020.
Application No. PCT/IB2020/050433 is a continuation-in-part of application No. 16/747,635, filed on Jan. 21, 2020, now Pat. No. 11,020,018.
(60) Provisional application No. 62/655,031, filed on Apr. 9, 2018.
(60) Provisional application No. 62/654,538, filed on Apr. 9, 2018.
(60) Provisional application No. 62/655,031, filed on Apr. 9, 2018.
(60) Provisional application No. 62/655,031, filed on Apr. 9, 2018.

(30) Foreign Application Priority Data
Jan. 3, 2019 (GB) 1900082.7
Jan. 19, 2019 (GB) 1900780.6

(51) Int. Cl. A61H 21/00; A61H 23/00

(52) U.S. Cl. CPC A61H 21/00; A61H 23/00; A61H 2201/1207

(57) ABSTRACT

A vibrating gastrointestinal capsule and method of use thereof for treating Parkinsonism in a subject, the method including: (a) providing a vibrating gastrointestinal capsule having a housing; a vibrating agitation mechanism adapted such that, in a vibrating mode of operation, the housing exerts vibrations on an environment surrounding the capsule; and a power supply disposed within the housing and adapted to power the vibrating agitation mechanism; (b) ingesting the capsule, by the subject; and (c) controlling the vibrating agitation mechanism such that at least a portion of the vibrating mode of operation occurs within the stomach and/or the small intestine of the subject.

### RELATED APPLICATIONS

The present application is a continuation of U.S. patent application Ser. No. 16/377,213, filed Apr. 7, 2019 and entitled VIBRATING CAPUSLE SYSTEM AND TREATMENT METHOD, which gains priority from U.S. Provisional Patent Application No. 62/654,538 filed Apr. 9, 2018 having the same title.

The present application is a continuation of U.S. patent application Ser. No. 17/044,258, filed Oct. 1, 2020 and entitled METHOD OF ENHANCING

ABSORPTION OF INGESTED MEDICAMENTS. U.S. patent application Ser. No. 17/044,258 is a National Phase application of PCT Patent Application No. PCT/IB2019/052866, filed Apr. 8, 2019 and entitled VIBRATING CAPSULE FOR ENHANCING ABSOPRTION OF INGESTED MEDICAMENTS. PCT Patent Application No. PCT/IB2019/052866 is a Continuation In Part of U.S. patent application Ser. No. 16/178,425 filed Nov. 1, 2018 and entitled METHOD OF ENHANCING ABSORPTION OF INGESTED MEDICAMENTS FOR TREATMENT OF PARKINSONISM and of U.S. patent application Ser. No. 16/185,044 filed Nov. 9, 2018, and entitled METHOD OF ENHANCING ABSORPTION OF INGESTED MEDICAMENTS FOR TREATMENT OF AN AILMENT OF THE GI TRACT. PCT Patent Application No. PCT/IB2019/052866, U.S. patent application Ser. No. 16/178,425, and U.S. patent application Ser. No. 16/185,044 all gain priority from U.S. Provisional Patent Application No. 62/655,031 filed Apr. 9, 2018 entitled METHOD OF ENHANCING ABSORPTION OF INGESTED MEDICAMENTS.

The present application is a continuation of U.S. patent application Ser. No. 17/419,187 filed Jun. 30, 2021, and entitled DEVICE AND METHOD FOR DELIVERING AN INGESTIBLE MEDICAMENT INTO THE GASTROINTESTINAL TRACT OF A USER. U.S. patent application Ser. No. 17/419,187 is a National Phase application of PCT Patent Application No. PCT/IB2020/050008, filed Jan. 2, 2020, having the same title, which gains priority from GB Patent Application No. 1900082.7, filed Jan. 3, 2019, and having the same title.

The present application is a continuation of U.S. patent application Ser. No. 17/246,643 filed May 2, 2021 and entitled DEVICE AND METHOD FOR DELIVERING A FLOWABLE INGESTIBLE MEDICAMENT INTO THE GASTROINTESTINAL TRACT OF A USER. U.S. patent application Ser. No. 17/246,643 is a Continuation In Part of U.S. patent application Ser. No. 16/747,635 filed Jan. 21, 2020, and of PCT Patent Application No. PCT/IB2020/050433 filed Jan. 21, 2020, both having the same title. PCT Patent Application No. PCT/IB2020/050433 gains priority from GB Patent Application No. 1900780.6 filed Jan. 21, 2019, and having the same title.

U.S. patent application Ser. Nos. 16/377,213; 17/044,258; 16/178,425; 16/185,044; 17/419,187; 17/246,643; and Ser. No. 16/747,635; US Provisional Patent Application Nos. 62/654,538; and 62/655,031; PCT Patent Application Numbers PCT/IB2019/052866; PCT/IB2020/050008; and PCT/IB2020/050433; and GB Patent Application Numbers 1900082.7 and 1900780.6 are all incorporated by reference as if fully set forth herein.

### FIELD OF THE INVENTION

The present invention relates in general to vibrating capsule systems including one or more vibrating capsules, and to treatment methods using such systems and capsules, and more particularly, to vibrating capsule systems and methods for treating Parkinsonism, enhancing the absorption to the bloodstream of an ingested medicament, and delivery of ingestible medicaments.

### SUMMARY OF THE INVENTION

In accordance with an embodiment of the present invention, there is provided a method of using a vibrating gastrointestinal capsule in the treatment of Parkinsonism in a subject, the method including:
(a) providing the vibrating gastrointestinal capsule, the vibrating gastrointestinal capsule having:
   a housing;
   a vibrating agitation mechanism adapted such that, in a first vibrating mode of operation, the housing exerts vibrations on an environment surrounding the vibrating gastrointestinal capsule; and
   a power supply disposed within the housing and adapted to power the vibrating agitation mechanism;
(b) ingesting the vibrating gastrointestinal capsule, by the subject;
(c) controlling or activating the vibrating agitation mechanism such that at least a portion of the first vibrating mode of operation occurs within a portion of a gastrointestinal tract of the subject, the portion consisting at most of a stomach, a small intestine, and large intestine.

In some embodiments, the portion of the gastrointestinal tract consists at most of the stomach and the small intestine. In some such embodiments, the portion of the gastrointestinal tract consists of the stomach. In other such embodiments, the portion of the gastrointestinal tract consists of the small intestine.

In some embodiments, the vibrating gastrointestinal capsule is adapted and/or dimensioned to transit the gastrointestinal tract.

In some embodiments, the vibrating gastrointestinal capsule includes, or is associated with, a control mechanism adapted to activate the vibrating agitation mechanism to operate in the first vibrating mode of operation.

In some embodiments, the treatment of Parkinsonism is, or includes, delaying an onset of Parkinsonism. In some embodiments, the treatment of Parkinsonism is, or includes, mitigating or retarding a development of Parkinsonism.

In some embodiments, the treatment of Parkinsonism is, or includes, managing a condition of Parkinsonism. In some embodiments, managing a condition of Parkinsonism includes effecting an increased absorption of a medicament used in the treatment of the Parkinsonism, thereby improving a therapeutic efficacy of the medicament. In some embodiments, managing a condition of Parkinsonism includes effecting an increased absorption of a medicament used in the treatment of the Parkinsonism, thereby enabling the use of a lower dosage of the medicament, optionally without impairing or diminishing therapeutic efficacy.

In some embodiments, the treatment of Parkinsonism is the treatment of Parkinson's disease.

In some embodiments, the first vibrating mode of operation is effected within the portion of the gastrointestinal tract so as to stimulate the enteric nervous system of the subject.

In some embodiments, the first vibrating mode of operation is effected within the portion of the gastrointestinal tract so as to induce at least one peristaltic wave in a wall of the gastrointestinal tract.

In some embodiments, the first vibrating mode of operation is effected within the portion of the gastrointestinal tract so as to increase peristalsis in a wall of the gastrointestinal tract. In some embodiments, the increasing of the peristalsis is effected so as to stimulate the enteric nervous system of the subject.

In some embodiments, the method further includes diagnosing a pre-disposition to Parkinsonism or Parkinson's disease in the subject, wherein the treatment of Parkinsonism is, or includes, delaying an onset of Parkinsonism.

In some embodiments, the method further includes timing at least one of the ingesting of the vibrating gastrointestinal capsule and the activating of the vibrating agitation mechanism such that the first vibrating mode of operation occurs during an absorption time of an ingested medicament within the gastrointestinal tract of the subject. In some embodiments, the absorption time is an estimated absorption time. In some embodiments, the absorption time is an actual absorption time.

In some embodiments, the timing is effected such that the ingesting of the vibrating gastrointestinal capsule transpires within 5 hours, within 4 hours, within 3.5 hours, within 3 hours, within 2.5 hours, within 2 hours, within 1.5 hours, within 1 hour, or within 0.5 hours of the ingesting of the medicament.

In some embodiments, the medicament includes a substance for treating Parkinsonism or Parkinson's disease. In some embodiments, the medicament includes at least one dopaminergic agent. In some embodiments, the medicament includes at least one catecholamine precursor. In some embodiments, the at least one catecholamine precursor includes a dopamine precursor. In some embodiments, the dopamine precursor includes at least one dopamine precursor agent such as (L)-3,4-dihydroxyphenylalanine. In some embodiments, the medicament includes N-methyl-N-(2-propynyl)-2-methyl-1-phenylethyl-2-amine.

In some embodiments, the capsule further includes a control mechanism adapted, in response to receipt of an activation input, to activate the vibrating agitation mechanism to operate in the first vibrating mode of operation.

In some embodiments, the capsule further includes at least one sensor adapted to provide the activation input.

In some embodiments, the at least one sensor includes an illumination sensor, and the receiving the at least one activation input includes receiving input indicating transition of the capsule from an illuminated environment to a dark environment.

In some embodiments, the at least one sensor includes a pressure sensor and the receiving the at least one activation input includes receiving input indicting pressure applied to the capsule, which pressure is indicative of the capsule moving through a pharynx of the subject.

In some embodiments, the at least one sensor includes a temperature sensor, and the receiving the at least one activation input includes receiving input indicating transition of the capsule from an area with ambient temperature to an area with a human body temperature.

In some embodiments, the at least one sensor includes an accelerometer, and the receiving the at least one activation input includes receiving the activation input in response to a detected activation motion carried out with the gastrointestinal capsule.

In some embodiments, the at least one sensor includes a moisture sensor, and the receiving the at least one activation input includes receiving input indicating transition of the capsule from a dry environment to a humid environment.

In some embodiments, the receipt of the activation input includes receiving the activation input from a control unit remote from the gastrointestinal capsule.

In some embodiments, the receiving the activation input includes receiving the activation input following the ingesting.

In some embodiments, the receiving the activation input includes receiving the activation input prior to the ingesting.

In some embodiments, the receiving the activation input additionally includes receiving a vibration protocol to be used by the control mechanism to control operation of the vibrating agitation mechanism.

In some embodiments, the vibrating agitation mechanism includes at least a radial agitation mechanism, and the controlling includes controlling the radial agitation mechanism, in the first vibrating mode of operation, to exert radial forces on the housing, in a radial direction with respect to a or the longitudinal axis of the housing, thereby to cause the vibrations exerted by the housing.

In some embodiments, the vibrating agitation mechanism includes at least an axial agitation mechanism, and wherein the control mechanism is adapted to control the axial agitation mechanism, in the first vibrating mode of operation, to exert axial forces on the housing, in an axial direction with respect to a or the longitudinal axis of the housing, thereby to cause the vibrations exerted by the housing.

In some embodiments, the control mechanism is adapted to control the vibrating agitation mechanism, in the first vibrating mode of operation, to exert radial forces on the housing in a radial direction with respect to a or the longitudinal axis of the housing and to exert axial forces on the housing in an axial direction with respect to a or the longitudinal axis of the housing, thereby to cause the vibrations exerted by the housing.

In some embodiments, the vibrating agitation mechanism includes a radial agitation mechanism adapted to exert the radial forces and a separate axial agitation mechanism adapted to exert the axial forces.

In some embodiments, the vibrating agitation mechanism includes a single agitation mechanism adapted to exert the radial forces and the axial forces.

In some embodiments, the control mechanism is adapted to control the vibrating agitation mechanism such that the vibrating mode of operation includes a plurality of cycles, each of the cycles including a vibration duration followed by a repose duration, wherein the housing exerts the vibrations during the vibration duration.

In some embodiments, the repose duration is greater than the vibration duration.

In some embodiments, the vibration duration is in the range of 0.1 second to 10 seconds, 1 second to 10 seconds, 1 second to 9 seconds, 2 seconds to 9 seconds, 3 seconds to 9 seconds, 3 seconds to 8 seconds, 3 seconds to 7 seconds, 3 seconds to 6 seconds, 4 seconds to 6 seconds, or 5 seconds to 6 seconds.

In some embodiments, the repose duration is in the range of 1 second to 180 seconds, 3 seconds to 180 seconds, 5 seconds to 180 seconds, 5 seconds to 150 seconds, seconds to 120 seconds, 8 seconds to 100 seconds, 8 seconds to 30 seconds, 10 seconds to 80 seconds, 10 seconds to 70 seconds, 10 seconds to 60 seconds, 10 seconds to 50 seconds, 10 seconds to 40 seconds, 10 seconds to 30 seconds, 10 seconds to 20 seconds, or 15 seconds to 20 seconds.

In some embodiments, a duration of each of the plurality of cycles is in the range of 1.1 seconds to 200 seconds, 5 seconds to 200 seconds, 10 seconds to 200 seconds, 10 seconds to 150 seconds, 10 seconds to 100 seconds, 10 seconds to 80 seconds, 10 seconds to 50 seconds, 10 seconds to 40 seconds, 10 seconds to 30 seconds, 15 seconds to 50 seconds, 15 seconds to 40 seconds, 15 seconds to 30 seconds, or 15 seconds to 25 seconds.

In some embodiments, the control mechanism is adapted to control the vibrating agitation mechanism such that a cumulative duration of the vibrating mode of operation is in the range of 1 hour to 12 hours, 2 hours to 10 hours, 2 hours to 8 hours, 2 hours to 6 hours, 2 hours to 4 hours, or 2 hours to 3 hours.

In some embodiments, in the first vibration mode of operation, the vibrating agitation mechanism is configured such that a net force exerted by the housing on the environment is in the range of 50 grams-force to 600 grams-force.

In some embodiments, in the first vibration mode of operation the vibrating agitation mechanism is configured to exert the forces on the housing to attain a vibrational frequency within a range of 10 Hz to 650 Hz, 15 Hz to 600 Hz, 20 Hz to 550 Hz, 30 Hz to 550 Hz, 50 Hz to 500 Hz, 70 Hz to 500 Hz, 100 Hz to 500 Hz, 130 Hz to 500 Hz, or 150 Hz to 500 Hz.

In some embodiments, the activating the vibrating agitation mechanism is effected such that at least a portion of the first vibrating mode of operation occurs within 6 hours, within 5 hours, within 4.5 hours, within 4 hours, within 3.5 hours, within 3 hours, within 2.5 hours, within 2 hours, within 1.5 hours, within 1 hour, or within 0.5 hours of the ingesting.

In accordance with another embodiment of the present invention, there is provided a vibrating gastrointestinal capsule for use in the treatment of Parkinsonism in a subject, the capsule including:
(a) a housing;
(b) a vibrating agitation mechanism adapted such that, in a first vibrating mode of operation, the housing exerts vibrations on an environment surrounding the vibrating gastrointestinal capsule; and
(c) a power supply disposed within the housing and adapted to power the vibrating agitation mechanism.

In some embodiments, the capsule further includes at least one of the structural features as described hereinabove.

In accordance with a further embodiment of the present invention, there is provided a method of using a vibrating gastrointestinal capsule in the treatment of Parkinsonism in a subject, the method including:
(a) providing the vibrating gastrointestinal capsule, the vibrating gastrointestinal capsule having:
   a housing;
   a vibrating agitation mechanism adapted such that, in a first vibrating mode of operation, the housing exerts vibrations on an environment surrounding the vibrating gastrointestinal capsule; and
   a power supply disposed within the housing and adapted to power the vibrating agitation mechanism;
(b) ingesting the vibrating gastrointestinal capsule, by the subject;
(c) activating the vibrating agitation mechanism such that at least a portion of the first vibrating mode of operation occurs within 6 hours, within 5 hours, within 4.5 hours, within 4 hours, within 3.5 hours, within 3 hours, within 2.5 hours, within 2 hours, within 1.5 hours, within 1 hour, or within 0.5 hours of the ingesting of the vibrating gastrointestinal capsule.

In accordance with an embodiment of the present invention, there is provided a vibrating ingestible capsule for promoting absorption of an ingested medicament into the blood stream, the vibrating ingestible capsule including:
a housing;
a vibrating agitation mechanism adapted such that, in a vibration mode of operation, the housing exerts vibrations on an environment surrounding the vibrating gastrointestinal capsule;
a power supply disposed within the housing and adapted to power the vibrating agitation mechanism; and
a control mechanism adapted to activate the vibrating agitation mechanism to be operative in the vibration mode of operation, the control mechanism adapted to control a timing or activation delay of the vibration mode of operation such that a first occurrence of the vibration mode of operation at least partially transpires within at least one of an estimated absorption time period and an actual absorption time period of the ingested medicament within the gastrointestinal tract of the subject.

In some embodiments, the vibrating ingestible capsule is devoid of a chamber for containing the ingested medicament, prior to ingestion thereof.

In some embodiments, operation of the vibrating agitation mechanism in the vibration mode of operation maintain the integrity of the housing.

In some embodiments, operation of the vibrating agitation mechanism in the vibration mode of operation maintain the integrity of the vibrating ingestible capsule.

In some embodiments, the control mechanism is further adapted to control a timing of the vibration mode of operation such that a second occurrence of the vibration mode of operation transpires while the capsule is within at least one of the large intestine and the colon of the subject.

In some such embodiments, the control mechanism is further adapted to control a timing of the vibration mode of operation such that between the first and the second occurrences of the vibration mode of operation, the vibration agitation mechanism is in a rest mode of operation.

In some embodiments, the control mechanism is adapted to activate the vibration agitation mechanism to be operative in the vibration mode of operation in response to receipt of an activation input.

In some embodiments, the vibrating ingestible capsule further includes at least one sensor adapted to provide the activation input.

In some embodiments, the at least one sensor includes an illumination sensor, and the receiving the at least one activation input includes receiving input indicating transition of the capsule from an illuminated environment to a dark environment.

In some embodiments, the at least one sensor includes a pressure sensor and the receiving the at least one activation input includes receiving input indicting pressure applied to the capsule, which pressure is indicative of the capsule moving through a pharynx of the subject.

In some embodiments, the at least one sensor includes a temperature sensor, and the receiving the at least one activation input includes receiving input indicating transition of the capsule from an area with ambient temperature to an area with a human body temperature.

In some embodiments, the at least one sensor includes an accelerometer, and the receiving the at least one activation input includes receiving the activation input in response to a detected activation motion carried out with the gastrointestinal capsule.

In some embodiments, the at least one sensor includes a moisture sensor, and the receiving the at least one activation input includes receiving input indicating transition of the capsule from a dry environment to a humid environment.

In some embodiments, the vibrating ingestible capsule is functionally associated with a control unit remote from the vibrating ingestible capsule, and the control mechanism is adapted to receive the activation input from the control unit.

In some embodiments, the control mechanism is adapted to receive the activation input following ingesting of the vibrating capsule.

In some embodiments, the control mechanism is adapted to receive the activation input prior to ingesting of the vibrating capsule.

In some embodiments, the control mechanism is adapted to receive the activation input by receiving a vibration protocol to be used by the control mechanism to control operation of the vibrating agitation mechanism.

In some embodiments, the vibrating agitation mechanism includes at least a radial agitation mechanism adapted, in the vibration mode of operation, to exert radial forces on the housing, in a radial direction with respect to a longitudinal axis of the housing, thereby to cause the vibrations exerted by the housing.

In some embodiments, the vibrating agitation mechanism includes at least an axial agitation mechanism adapted, in the vibration mode of operation, to exert axial forces on the housing, in an axial direction with respect to a longitudinal axis of the housing, thereby to cause the vibrations exerted by the housing.

In some embodiments, the vibrating agitation mechanism includes a radial agitation mechanism adapted to exert the radial forces and a separate axial agitation mechanism adapted to exert the axial forces.

In some embodiments, the vibrating agitation mechanism includes a single agitation mechanism adapted to exert the radial forces and the axial forces.

In some embodiments, the control mechanism is adapted to control the vibrating agitation mechanism such that the vibrating mode of operation includes a plurality of cycles, each of the cycles including a vibration duration followed by a repose duration, wherein the housing exerts the vibrations during the vibration duration.

In some embodiments, the repose duration is greater than the vibration duration.

In some embodiments, the vibration duration is in the range of 0.1 second to 10 seconds, 1 second to 10 seconds, 1 second to 9 seconds, 2 seconds to 9 seconds, 3 seconds to 9 seconds, 3 seconds to 8 seconds, 3 seconds to 7 seconds, 3 seconds to 6 seconds, 4 seconds to 6 seconds, or 5 seconds to 6 seconds.

In some embodiments, the repose duration is in the range of 1 second to 180 seconds, 3 seconds to 180 seconds, 5 seconds to 180 seconds, 5 seconds to 150 seconds, seconds to 120 seconds, 8 seconds to 100 seconds, 8 seconds to 30 seconds, 10 seconds to 80 seconds, 10 seconds to 70 seconds, 10 seconds to 60 seconds, 10 seconds to 50 seconds, 10 seconds to 40 seconds, 10 seconds to 30 seconds, 10 seconds to 20 seconds, or 15 seconds to 20 seconds.

In some embodiments, a duration of each of the plurality of cycles is in the range of 1.1 seconds to 200 seconds, 5 seconds to 200 seconds, 10 seconds to 200 seconds, 10 seconds to 150 seconds, 10 seconds to 100 seconds, 10 seconds to 80 seconds, 10 seconds to 50 seconds, 10 seconds to 40 seconds, 10 seconds to 30 seconds, 15 seconds to 50 seconds, 15 seconds to 40 seconds, 15 seconds to 30 seconds, or 15 seconds to 25 seconds.

In some embodiments, the control mechanism is adapted to control the vibrating agitation mechanism such that a cumulative duration of the vibrating mode of operation is in the range of 1 hour to 12 hours, 2 hours to 10 hours, 2 hours to 8 hours, 2 hours to 6 hours, 2 hours to 4 hours, or 2 hours to 3 hours.

In some embodiments, in the first vibration mode of operation, the vibrating agitation mechanism is configured such that a net force exerted by the housing on the environment is in the range of 50 grams-force to 600 grams-force.

In some embodiments, in the first vibration mode of operation the vibrating agitation mechanism is configured to exert the forces on the housing to attain a vibrational frequency within a range of 10 Hz to 650 Hz, 15 Hz to 600 Hz, 20 Hz to 550 Hz, 30 Hz to 550 Hz, 50 Hz to 500 Hz, 70 Hz to 500 Hz, 100 Hz to 500 Hz, 130 Hz to 500 Hz, or 150 Hz to 500 Hz.

In accordance with another embodiment of the present invention, there is provided a method of using a vibrating gastrointestinal capsule in coordination with an ingestible medicament, the method including:
(a) providing the vibrating gastrointestinal capsule, the capsule including:
   a housing;
   a vibrating agitation mechanism adapted such that, in a vibration mode of operation, the housing exerts vibrations on an environment surrounding the vibrating gastrointestinal capsule;
   a power supply disposed within the housing and adapted to power the vibrating agitation mechanism; and
   a control mechanism adapted to activate the vibrating agitation mechanism to operate in the vibration mode of operation;
(b) ingesting the ingestible medicament;
(c) ingesting the vibrating gastrointestinal capsule; and
(d) controlling at least one of a time of the ingesting of the vibrating gastrointestinal capsule and a timing or activation delay of the vibration mode of operation, such that a first occurrence of the vibration mode of operation at least partially transpires within at least one of an estimated absorption time period and an actual absorption time period of the ingestible medicament within the gastrointestinal tract of the subject.

In some embodiments, the controlling comprises controlling the operation of the vibrating agitation mechanism in the vibration mode of operation to maintain the integrity of the housing.

In some embodiments, the controlling comprises controlling the operation of the vibrating agitation mechanism in the vibration mode of operation to maintain the integrity of the vibrating ingestible capsule.

In some embodiments, the controlling further includes controlling a timing of the vibration mode of operation such that a second occurrence of the vibration mode of operation transpires while the capsule is within at least one of the large intestine and the colon of the subject.

In some embodiments, the controlling further includes controlling a timing of the vibration mode of operation such that between the first and the second occurrences of the vibration mode of operation, the vibration agitation mechanism is in a rest mode of operation.

In some embodiments, the controlling is effected such that the vibration mode of operation at least partially transpires during the actual absorption time.

In some embodiments, the actual absorption time period occurs when the ingestible medicament is disposed in a stomach of the subject. In some embodiments, the actual absorption time period occurs when the ingestible medicament is disposed in a small intestine of the subject. In some embodiments, the actual absorption time period occurs when the ingestible medicament is disposed in a large intestine of the subject.

In some embodiments, the controlling of is effected such that the vibration mode of operation at least partially transpires during the estimated absorption time. In some embodiments, the estimated absorption time is within a range of 0.5 to 1.5 hours. In some embodiments, the estimated absorption time is within a range of 1.0 to 5 hours. In some embodiments, the estimated absorption time is within a range of 0.5 to 5 hours. In some embodiments, the estimated absorption time is within a range of 4 to 30 hours.

In some embodiments, the ingesting of the vibrating gastrointestinal capsule transpires within 4 hours, within 3.5 hours, within 3 hours, within 2.5 hours, within 2 hours, within 1.5 hours, within 1 hour, or within 0.5 hours of, or after, the ingesting of the ingestible medicament.

In some embodiments, the ingesting of the vibrating gastrointestinal capsule is simultaneous with the ingesting of the ingestible medicament.

In some embodiments, the method further includes timing the vibration mode of operation to at least partially transpire within 5 hours, within 4 hours, within 3.5 hours, within 3 hours, within 2.5 hours, within 2 hours, within 1.5 hours, within 1 hour, or within 0.5 hours of the ingesting of the ingestible medicament.

In some embodiments, the ingestible medicament is at least partially absorbable in a stomach of a subject or in the stomach of the subject. In some embodiments, the ingestible medicament is at least partially absorbable in a small intestine of a subject or in the small intestine of the subject.

In some embodiments, the ingestible medicament includes an ingestible medicament for treatment of Parkinsonism. In some embodiments, the ingestible medicament for treatment of Parkinsonism includes levodopa. In some embodiments, the ingestible medicament for treatment of Parkinsonism includes at least one dopaminergic agent. In some embodiments, the ingestible medicament for treatment of Parkinsonism includes at least one catecholamine precursor. In some embodiments, the at least one catecholamine precursor includes a dopamine precursor. In some such embodiments, the dopamine precursor includes at least one dopamine precursor agent such as (L)-3,4-dihydroxyphenylalanine. In some embodiments, the ingestible medicament for treatment of Parkinsonism includes N-methyl-N-(2-propynyl)-2-methyl-1-phenylethyl-2-amine. In some embodiments, the ingestible medicament for treatment of Parkinsonism includes tyrosine hydroxylase. In some embodiments, the ingestible medicament for treatment of Parkinsonism includes apomorphine. In some embodiments, the ingestible medicament for treatment of Parkinsonism includes at least one anticholinergic agent. In some embodiments, the ingestible medicament for treatment of Parkinsonism includes at least one agent selected to antagonize at least one cholinergic receptor. In some embodiments, the ingestible medicament for treatment of Parkinsonism includes at least one of benzhexol and orphenadrine. In some embodiments, the ingestible medicament for treatment of Parkinsonism includes at least one selective allosteric potentiator of metabotropic glutamate receptor 4 (mGluR4), optionally N-phenyl-7-(hydroxylimino)cyclopropa[b] chromen-1a-carboxamide.

In some embodiments, the ingestible medicament for treatment of Parkinsonism is adapted to delay an onset of Parkinsonism. In some embodiments, the ingestible medicament for treatment of Parkinsonism is adapted to mitigate or retard a development of Parkinsonism. In some embodiments, the ingestible medicament for treatment of Parkinsonism is adapted to manage a condition of Parkinsonism.

In some embodiments, the ingestible medicament includes an ingestible medicament for treatment of an ailment of the GI tract.

In some embodiments, the ingestible medicament for treatment of an ailment of the GI tract includes an ingestible medicament for treatment of constipation. In some embodiments, the ingestible medicament for treatment of an ailment of the GI tract includes an ingestible medicament for treatment of Crohn's disease. In some embodiments, the ingestible medicament for treatment of an ailment of the GI tract includes an ingestible medicament for treatment of gastroparesis. In some embodiments, the ingestible medicament for treatment of an ailment of the GI tract includes an ingestible medicament for treatment of irritable bowel syndrome (IBS). In some embodiments, the ingestible medicament for treatment of an ailment of the GI tract includes an ingestible medicament for treatment of diarrhea or loose bowel movements. In some embodiments, the ingestible medicament for treatment of an ailment of the GI tract includes an ingestible medicament for treatment of colitis. In some embodiments, the ingestible medicament for treatment of an ailment of the GI tract includes an ingestible medicament for treatment of Hirschsprung's disease. In some embodiments, the ingestible medicament for treatment of an ailment of the GI tract includes an ingestible medicament for treatment of dyspepsia. In some embodiments, the ingestible medicament for treatment of an ailment of the GI tract includes an ingestible medicament for treatment of dysphagia.

In some embodiments, the ingestible medicament for treatment of an ailment of the GI tract includes at least one osmotic agent, such as magnesium citrate, magnesium hydroxide, polyethylene glycol, or sodium phosphate. In some embodiments, the ingestible medicament for treatment of an ailment of the GI tract includes MiraLAX®. In some embodiments, the ingestible medicament for treatment of an ailment of the GI tract includes at least one contraction stimulating agent, such as bisacodyl or senna. In some embodiments, the ingestible medicament for treatment of an ailment of the GI tract includes at least one of Correctol, Ducodyl, Dulcolax, Senexon, and Senokot. In some embodiments, the ingestible medicament for treatment of an ailment of the GI tract includes at least one stool softening agent, such as docusate sodium. In some embodiments, the ingestible medicament for treatment of an ailment of the GI tract includes Colace. In some embodiments, the ingestible medicament for treatment of an ailment of the GI tract includes linaclotide. In some embodiments, the ingestible medicament for treatment of an ailment of the GI tract includes lactulose. In some embodiments, the ingestible medicament for treatment of an ailment of the GI tract includes lubiprostone. In some embodiments, the ingestible medicament for treatment of an ailment of the GI tract includes plecanatide. In some embodiments, the ingestible medicament for treatment of an ailment of the GI tract includes prucaltride. In some embodiments, the ingestible medicament for treatment of an ailment of the GI tract includes a fluid absorption agent, such as loperamide or bismuth subsalicylate.

In some embodiments, the medicament for treatment of an ailment of the GI tract is adapted to reduce constipation. In some embodiments, the medicament for treatment of an ailment of the GI tract is adapted to change the consistency of stool. In some embodiments, the medicament for treatment of an ailment of the GI tract is adapted to reduce straining while defecating. In some embodiments, the medicament for treatment of an ailment of the GI tract is adapted to reduce a sensation of abdominal bloating. In some embodiments, the medicament for treatment of an ailment of the GI tract is adapted to affect the microbiome of at least a portion of the GI tract. In some embodiments, the medicament for treatment of an ailment of the GI tract is adapted to manage a condition of the ailment of the GI tract.

In some embodiments, the vibration mode of operation at least partially transpiring within at least one of an estimated absorption time period and an actual absorption time period of the ingestible medicament within the gastrointestinal tract of the subject the effects an increased absorption of the ingestible medicament, thereby improving a therapeutic efficacy of the medicament.

In some embodiments, the vibration mode of operation at least partially transpiring within at least one of an estimated absorption time period and an actual absorption time period of the ingestible medicament within the gastrointestinal tract of the subject effects an increased absorption of the ingestible medicamen, thereby enabling the use of a lower dosage of the medicament, optionally without impairing or diminishing therapeutic efficacy.

In some embodiments, the vibration mode of operation is effected within the gastrointestinal tract so as to stimulate the enteric nervous system of the subject. In some embodiments, the vibration mode of operation is effected within the gastrointestinal tract so as to induce at least one peristaltic wave in a wall of the gastrointestinal tract. In some embodiments, the vibration mode of operation is effected within the gastrointestinal tract so as to effect increasing peristalsis in a wall of the gastrointestinal tract. In some embodiments, increasing peristalsis is effected so as to stimulate the enteric nervous system of the subject.

In some embodiments, the vibrating gastrointestinal capsule is adapted and/or dimensioned to transit the gastrointestinal tract of the subject or of a subject.

In some embodiments, the capsule further includes a control mechanism adapted, in response to receipt of an activation input, to activate the vibrating agitation mechanism to operate in the vibration mode of operation.

In some embodiments, the capsule further includes at least one sensor adapted to provide the activation input.

In some embodiments, the at least one sensor includes an illumination sensor, and the receiving the at least one activation input includes receiving input indicating transition of the capsule from an illuminated environment to a dark environment.

In some embodiments, the at least one sensor includes a pressure sensor and the receiving the at least one activation input includes receiving input indicting pressure applied to the capsule, which pressure is indicative of the capsule moving through a pharynx of the subject.

In some embodiments, the at least one sensor includes a temperature sensor, and the receiving the at least one activation input includes receiving input indicating transition of the capsule from an area with ambient temperature to an area with a human body temperature.

In some embodiments, the at least one sensor includes an accelerometer, and the receiving the at least one activation input includes receiving the activation input in response to a detected activation motion carried out with the gastrointestinal capsule.

In some embodiments, the at least one sensor includes a moisture sensor, and the receiving the at least one activation input includes receiving input indicating transition of the capsule from a dry environment to a humid environment.

In some embodiments, the receipt of the activation input includes receiving the activation input from a control unit remote from the gastrointestinal capsule. In some embodiments, receiving the activation input includes receiving the activation input following the ingesting. In some embodiments, receiving the activation input includes receiving the activation input prior to the ingesting. In some embodiments, receiving the activation input additionally includes receiving a vibration protocol to be used by the control mechanism to control operation of the vibrating agitation mechanism.

In some embodiments, the vibrating agitation mechanism includes at least a radial agitation mechanism, and the controlling includes controlling the radial agitation mechanism, in the vibration mode of operation, to exert radial forces on the housing, in a radial direction with respect to a or the longitudinal axis of the housing, thereby to cause the vibrations exerted by the housing.

In some embodiments, the vibrating agitation mechanism includes at least an axial agitation mechanism, and wherein the control mechanism is adapted to control the axial agitation mechanism, in the vibration mode of operation, to exert axial forces on the housing, in an axial direction with respect to a or the longitudinal axis of the housing, thereby to cause the vibrations exerted by the housing.

In some embodiments, the control mechanism is adapted to control the vibrating agitation mechanism, in the vibration mode of operation, to exert radial forces on the housing in a radial direction with respect to a or the longitudinal axis of the housing and to exert axial forces on the housing in an axial direction with respect to a or the longitudinal axis of the housing, thereby to cause the vibrations exerted by the housing.

In some embodiments, the vibrating agitation mechanism includes a radial agitation mechanism adapted to exert the radial forces and a separate axial agitation mechanism adapted to exert the axial forces.

In some embodiments, the vibrating agitation mechanism includes a single agitation mechanism adapted to exert the radial forces and the axial forces.

In some embodiments, the control mechanism is adapted to control the vibrating agitation mechanism such that the vibrating mode of operation includes a plurality of cycles, each of the cycles including a vibration duration followed by a repose duration, wherein the housing exerts the vibrations during the vibration duration.

In some embodiments, the repose duration is greater than the vibration duration.

In some embodiments, the vibration duration is in the range of 0.1 second to 10 seconds, 1 second to 10 seconds, 1 second to 9 seconds, 2 seconds to 9 seconds, 3 seconds to 9 seconds, 3 seconds to 8 seconds, 3 seconds to 7 seconds, 3 seconds to 6 seconds, 4 seconds to 6 seconds, or 5 seconds to 6 seconds.

In some embodiments, the repose duration is in the range of 1 second to 180 seconds, 3 seconds to 180 seconds, 5 seconds to 180 seconds, 5 seconds to 150 seconds, seconds to 120 seconds, 8 seconds to 100 seconds, 8 seconds to 30 seconds, 10 seconds to 80 seconds, 10 seconds to 70 seconds, 10 seconds to 60 seconds, 10 seconds to 50 seconds, 10 seconds to 40 seconds, 10 seconds to 30 seconds, 10 seconds to 20 seconds, or 15 seconds to 20 seconds.

In some embodiments, a duration of each of the plurality of cycles is in the range of 1.1 seconds to 200 seconds, 5 seconds to 200 seconds, 10 seconds to 200 seconds, 10 seconds to 150 seconds, 10 seconds to 100 seconds, 10 seconds to 80 seconds, 10 seconds to 50 seconds, 10 seconds to 40 seconds, 10 seconds to 30 seconds, 15 seconds to 50 seconds, 15 seconds to 40 seconds, 15 seconds to 30 seconds, or 15 seconds to 25 seconds.

In some embodiments, the control mechanism is adapted to control the vibrating agitation mechanism such that a cumulative duration of the vibrating mode of operation is in the range of 1 hour to 12 hours, 2 hours to 10 hours, 2 hours to 8 hours, 2 hours to 6 hours, 2 hours to 4 hours, or 2 hours to 3 hours.

In some embodiments, in the first vibration mode of operation, the vibrating agitation mechanism is configured such that a net force exerted by the housing on the environment is in the range of 50 grams-force to 600 grams-force.

In some embodiments, in the first vibration mode of operation the vibrating agitation mechanism is configured to exert the forces on the housing to attain a vibrational frequency within a range of 10 Hz to 650 Hz, 15 Hz to 600 Hz, 20 Hz to 550 Hz, 30 Hz to 550 Hz, 50 Hz to 500 Hz, 70 Hz to 500 Hz, 100 Hz to 500 Hz, 130 Hz to 500 Hz, or 150 Hz to 500 Hz.

In accordance with a further embodiment of the present invention, there is provided a method of using a vibrating gastrointestinal capsule in coordination with an ingestible medicament, the method including:
(a) providing the vibrating gastrointestinal capsule, the capsule including:
   a housing;
   a vibrating agitation mechanism adapted such that, in a vibration mode of operation, the housing exerts vibrations on an environment surrounding the vibrating gastrointestinal capsule;
   a power supply disposed within the housing and adapted to power the vibrating agitation mechanism; and
   a control mechanism adapted to activate the vibrating agitation mechanism to operate in the vibration mode of operation;
(b) ingesting the vibrating gastrointestinal capsule;
(c) ingesting the ingestible medicament; and
(d) controlling at least one of a time of ingesting the vibrating gastrointestinal capsule and a timing of the vibration mode of operation such that a first occurrence of the vibration mode at least partially transpires within a particular time period with respect to the ingesting of the ingestible medicament.

In some embodiments, the controlling comprises controlling the operation of the vibrating agitation mechanism in the vibration mode of operation to maintain the integrity of the housing.

In some embodiments, the controlling comprises controlling the operation of the vibrating agitation mechanism in the vibration mode of operation to maintain the integrity of the vibrating ingestible capsule.

In some embodiments, the controlling further includes controlling a timing of the vibration mode of operation such that a second occurrence of the vibration mode of operation transpires while the capsule is within at least one of the large intestine and the colon of the subject.

In some embodiments, the controlling further includes controlling a timing of the vibration mode of operation such that between the first and the second occurrences of the vibration mode of operation, the vibration agitation mechanism is in a rest mode of operation.

In some embodiments, the particular time period is within 5 hours, within 4 hours, within 3.5 hours, within 3 hours, within 2.5 hours, within 2 hours, within 1.5 hours, within 1 hour, or within 0.5 hours of the ingesting of the ingestible medicament.

In some embodiments, controlling includes both controlling the time of ingesting the vibrating gastrointestinal capsule and the timing of the vibration mode of operation.

In some embodiments, controlling is effected so as to improve absorption of the ingestible medicament within a gastrointestinal tract of a subject.

In accordance with another embodiment of the present invention, there is provided a method of using a vibrating gastrointestinal capsule in coordination with an ingestible medicament, the method including:
(a) providing the vibrating gastrointestinal capsule, the capsule including:
   a housing;
   a vibrating agitation mechanism adapted such that, in a vibration mode of operation, the housing exerts vibrations on an environment surrounding the vibrating gastrointestinal capsule;
   a power supply disposed within the housing and adapted to power the vibrating agitation mechanism; and
   a control mechanism adapted to activate the vibrating agitation mechanism to operate in the vibration mode of operation;
(b) ingesting the ingestible medicament;
(c) ingesting the vibrating gastrointestinal capsule; and
(d) controlling at least one of a time of the ingesting of the vibrating gastrointestinal capsule and a timing or activation delay of the vibration mode of operation, such that a first occurrence of the vibration mode of operation at least partially transpires within an estimated absorption time period of the ingestible medicament within the gastrointestinal tract of the subject.

In some embodiments, the controlling comprises controlling the operation of the vibrating agitation mechanism in the vibration mode of operation to maintain the integrity of the housing.

In some embodiments, the controlling comprises controlling the operation of the vibrating agitation mechanism in the vibration mode of operation to maintain the integrity of the vibrating ingestible capsule.

In some embodiments, the controlling further includes controlling a timing of the vibration mode of operation such that a second occurrence of the vibration mode of operation transpires while the capsule is within at least one of the large intestine and the colon of the subject.

In some embodiments, the controlling further includes controlling a timing of the vibration mode of operation such that between the first and the second occurrences of the vibration mode of operation, the vibration agitation mechanism is in a rest mode of operation.

In accordance with a further embodiment of the present invention, there is provided a method of using a vibrating gastrointestinal capsule in coordination with an ingestible medicament, the method including:
(a) providing the vibrating gastrointestinal capsule, the capsule including:
   a housing;
   a vibrating agitation mechanism adapted such that, in a vibration mode of operation, the housing exerts vibrations on an environment surrounding the vibrating gastrointestinal capsule;
   a power supply disposed within the housing and adapted to power the vibrating agitation mechanism; and
   a control mechanism adapted to activate the vibrating agitation mechanism to operate in the vibration mode of operation;
(b) ingesting the ingestible medicament;
(c) ingesting the vibrating gastrointestinal capsule; and
(d) controlling at least one of a time of the ingesting of the vibrating gastrointestinal capsule and a timing or activation delay of the vibration mode of operation, such that a first occurrence of the vibration mode of operation at least partially transpires within an actual absorption time period of the ingestible medicament within the gastrointestinal tract of the subject.

In some embodiments, the controlling comprises controlling the operation of the vibrating agitation mechanism in the vibration mode of operation to maintain the integrity of the housing.

In some embodiments, the controlling comprises controlling the operation of the vibrating agitation mechanism in the vibration mode of operation to maintain the integrity of the vibrating ingestible capsule.

In some embodiments, the controlling further includes controlling a timing of the vibration mode of operation such that a second occurrence of the vibration mode of operation transpires while the capsule is within at least one of the large intestine and the colon of the subject.

In some embodiments, the controlling further includes controlling a timing of the vibration mode of operation such that between the first and the second occurrences of the vibration mode of operation, the vibration agitation mechanism is in a rest mode of operation.

In accordance with yet another embodiment of the present invention, there is provided a kit for promoting absorption of an ingested medicament into the blood stream, the kit including:
a medicament to be ingested; and
vibrating ingestible capsule including:
   a vibrating agitation mechanism adapted such that, in a vibration mode of operation, the housing exerts vibrations on an environment surrounding the vibrating gastrointestinal capsule;
   a power supply disposed within the housing and adapted to power the vibrating agitation mechanism; and
   a control mechanism adapted to activate the vibrating agitation mechanism to operative in the vibration mode of operation, the control mechanism adapted to control a timing or activation delay of the vibration mode of operation such that a first occurrence of the vibration mode of operation at least partially transpires within at least one of an estimated absorption time period and an actual absorption time period of the ingested medicament within the gastrointestinal tract of the subject.

In some embodiments, the vibrating ingestible capsule is devoid of a chamber for containing the medicament to be ingested.

In accordance with a further embodiment of the present invention, there is provided use of a vibrating ingestible capsule for promoting absorption of an ingested medicament into the blood stream, the vibrating ingestible capsule including:
a housing;
a vibrating agitation mechanism adapted such that, in a vibration mode of operation, the housing exerts vibrations on an environment surrounding the vibrating gastrointestinal capsule;
a power supply disposed within the housing and adapted to power the vibrating agitation mechanism; and
a control mechanism adapted to activate the vibrating agitation mechanism to operative in the vibration mode of operation, the control mechanism adapted to control a timing or activation delay of the vibration mode of operation such that a first occurrence of the vibration mode of operation at least partially transpires within at least one of an estimated absorption time period and an actual absorption time period of the ingested medicament within the gastrointestinal tract of the subject.

In accordance with an embodiment of the present invention, there is provided a device for delivering an ingestible medicament of a medicament tablet into the gastrointestinal tract of a user, the device including:
a vibrating ingestible capsule including:
   a housing;
   a vibrating agitator disposed within the housing and adapted such that, in a vibration mode of operation, the housing exerts vibrations on an environment surrounding the vibrating gastrointestinal capsule;
   a power supply disposed within the housing and adapted to power the vibrating agitator; and
   a control element adapted to activate the vibrating agitator to be operative in the vibration mode of operation; and
a hollow medicament compartment housing, associated with the housing, and having at least one aperture formed in the medicament compartment housing,
wherein a hollow of the medicament compartment housing is dimensioned and configured to have the medicament tablet disposed therein, and
wherein the at least one aperture is dimensioned and configured to enable fluid communication between an environment surrounding the medicament compartment housing and the hollow.

In some embodiments, the at least one aperture is dimensioned and configured such that, when the medicament tablet is disposed within the hollow and the device is in the gastrointestinal tract of the user, the ingestible medicament of the medicament tablet enters the environment surrounding the medicament compartment housing for delivery thereof to the body of the user.

In some embodiments, at least one vibration parameter of the vibrating ingestible capsule is set so as to promote absorption of the ingestible medicament into the bloodstream of the user. In some embodiments, the at least one vibration parameter includes at least one of a vibration frequency, a cumulative vibration duration, a number of vibration cycles per time unit, a duration of a vibration duration within a vibration cycle, a duration of a repose duration within a vibration cycle, a total duration of a single vibration cycle, and a net force exerted by the housing on the environment.

In some embodiments, the device further includes at least one biasing mechanism adapted, when the medicament tablet is disposed within the hollow of the medicament compartment housing, to bias the medicament tablet toward the housing.

In some embodiments, the at least one biasing mechanism includes at least one longitudinal biasing mechanism adapted to bias the medicament tablet by application of pressure along a longitudinal axis of the medicament tablet. In some embodiments, the at least one biasing mechanism includes at least one radial biasing mechanism adapted to bias the medicament tablet by application of radial pressure along a perimeter of the medicament tablet.

In some embodiments, the housing of the vibrating ingestible capsule includes an attachment mechanism and the medicament compartment housing includes a corresponding attachment mechanism, for mutual attachment of the vibrating ingestible capsule to the hollow medicament compartment housing.

In some embodiments, the control element is adapted to control a timing or activation delay of the vibration mode of operation such that the vibration mode of operation at least partially transpires within at least one of an estimated absorption time period and an actual absorption time period of the ingestible medicament released from the medicament tablet within the gastrointestinal tract of the user.

In some embodiments, the control element is adapted to activate the vibrating agitator to be operative in the vibration mode of operation in response to receipt of an activation input.

In some embodiments, the device further includes at least one sensor adapted to provide the activation input to the control element. In some embodiments, the at least one sensor forms part of the vibrating ingestible capsule.

In some embodiments, the at least one sensor includes an illumination sensor, adapted to provide an input indicating transition of the device from an illuminated environment to a dark environment.

In some embodiments, the at least one sensor includes a pressure sensor, adapted to provide an input indicating pressure applied to the device, which pressure is indicative of the device moving through a pharynx of the user.

In some embodiments, the at least one sensor includes a temperature sensor, adapted to provide an input indicating transition of the device from an area with ambient temperature to an area with a human body temperature.

In some embodiments, the at least one sensor includes an accelerometer, adapted to provide an input in response to a detected activation motion carried out with the device.

In some embodiments, the at least one sensor includes a moisture sensor, adapted to provide an input indicating transition of the device from a dry environment to a humid environment.

In some embodiments, the device is functionally associated with a control unit remote from the device, and wherein the control element is adapted to receive the activation input from the control unit.

In some embodiments, the control element is adapted to receive the activation input following ingesting of the device.

In some embodiments, the control element is adapted to receive the activation input prior to ingesting of the device.

In some embodiments, the control element is adapted to receive the activation input by receiving a vibration protocol to be used by the control element to control operation of the vibrating agitator.

In some embodiments, the vibrating agitator includes at least a radial agitator adapted, in the vibration mode of operation, to exert radial forces on the housing, in a radial direction with respect to a or the longitudinal axis of the housing, thereby to cause the vibrations exerted by the housing.

In some embodiments, the vibrating agitator includes at least an axial agitator adapted, in the vibration mode of operation, to exert axial forces on the housing, in an axial direction with respect to a or the longitudinal axis of the housing, thereby to cause the vibrations exerted by the housing.

In some embodiments, the vibrating agitator includes a radial agitator adapted, in the vibration mode of operation, to exert radial forces on the housing, in a radial direction with respect to a or the longitudinal axis of the housing, and a separate axial agitator adapted, in the vibration mode of operation, to exert axial forces on the housing, in an axial direction with respect to a or the longitudinal axis of the housing.

In some embodiments, the vibrating agitator includes a single agitator adapted, in the vibration mode of operation, to exert radial forces on the housing in a radial direction with respect to a or the longitudinal axis of the housing, and to exert axial forces on the housing, in an axial direction with respect to the longitudinal axis of the housing.

In some embodiments, the control element is adapted to control the vibrating agitator such that the vibrating mode of operation includes a plurality of cycles, each of the cycles including a vibration duration followed by a repose duration, wherein the housing exerts the vibrations during the vibration duration.

In some embodiments, the repose duration is greater than the vibration duration.

In some embodiments, the vibration duration is in the range of 0.1 second to 10 seconds, 1 second to 10 seconds, 1 second to 9 seconds, 2 seconds to 9 seconds, 3 seconds to 9 seconds, 3 seconds to 8 seconds, 3 seconds to 7 seconds, 3 seconds to 6 seconds, 4 seconds to 6 seconds, or 5 seconds to 6 seconds.

In some embodiments, the repose duration is in the range of 1 second to 180 seconds, 3 seconds to 180 seconds, 5 seconds to 180 seconds, 5 seconds to 150 seconds, seconds to 120 seconds, 8 seconds to 100 seconds, 8 seconds to 30 seconds, 10 seconds to 80 seconds, 10 seconds to 70 seconds, 10 seconds to 60 seconds, 10 seconds to 50 seconds, 10 seconds to 40 seconds, 10 seconds to 30 seconds, 10 seconds to 20 seconds, or 15 seconds to 20 seconds.

In some embodiments, a duration of each of the plurality of cycles is in the range of 1.1 seconds to 200 seconds, 5 seconds to 200 seconds, 10 seconds to 200 seconds, 10 seconds to 150 seconds, 10 seconds to 100 seconds, 10 seconds to 80 seconds, 10 seconds to 50 seconds, 10 seconds to 40 seconds, 10 seconds to 30 seconds, 15 seconds to 50 seconds, 15 seconds to 40 seconds, 15 seconds to 30 seconds, or 15 seconds to 25 seconds.

In some embodiments, the control element is adapted to control the vibrating agitator such that a cumulative duration of the vibrating mode of operation is in the range of 1 hour to 12 hours, 2 hours to 10 hours, 2 hours to 8 hours, 2 hours to 6 hours, 2 hours to 4 hours, or 2 hours to 3 hours.

In some embodiments, the vibrating agitator is configured to exert forces on the housing of the vibrating ingestible capsule, such that a net force exerted by the housing on the environment thereof is in the range of 50 grams force (gf) to 600 gf, 50 gf to 550 gf, 100 gf to 550 gf, 100 gf to 500 gf, 150 gf to 500 gf, 200 gf to 500 gf, or 200 gf to 450 gf.

In some embodiments, the vibrating agitator is configured to exert the forces on the housing to attain a housing vibrational frequency within a range of 10 Hz to 650 Hz, 15 Hz to 600 Hz, 20 Hz to 550 Hz, 30 Hz to 550 Hz, 50 Hz to 500 Hz, 70 Hz to 500 Hz, 100 Hz to 500 Hz, 130 Hz to 500 Hz, or 150 Hz to 500 Hz.

In some embodiments, the medicament compartment housing is at least partially attached to the vibrating ingestible capsule. In some embodiments, the medicament compartment housing is attached to the vibrating ingestible capsule. In some embodiments, the medicament compartment housing is fixedly attached to the vibrating ingestible capsule.

In some embodiments, the medicament compartment housing is attached to the vibrating ingestible capsule by snap fit engagement. In some embodiments, the medicament compartment housing is attached to the vibrating ingestible capsule by threaded engagement. In some embodiments, the medicament compartment housing is attached to the vibrating ingestible capsule by adhering. In some embodiments, the medicament compartment housing is attached to the vibrating ingestible capsule by soldering.

In some embodiments, the medicament compartment housing at least partially envelops the vibrating ingestible capsule. In some embodiments, the medicament compartment housing fully envelops the vibrating ingestible capsule. In some embodiments, the medicament compartment housing includes a hollow capsule including the at least one aperture, having the vibrating ingestible capsule disposed therein.

In some embodiments, the hollow of the medicament compartment housing has a volume in the range of 200 mm³ to 800 mm³, 300 mm³ to 700 mm³, or 400 mm³ to 600 mm³.

In some embodiments, the at least one aperture is dimensioned and configured such that the medicament tablet, while whole, cannot be removed from the hollow.

In some embodiments, wherein the at least one aperture is dimensioned and configured to enable insertion of the medicament tablet, via the at least one aperture, into the hollow.

In some embodiments, in use, the at least one biasing mechanism is adapted to continue biasing the medicament tablet toward the housing, while the ingestible medicament from the medicament tablet is delivered to the environment surrounding the hollow.

In some embodiments, the medicament compartment housing is biodegradable. the medicament compartment housing is digestible by the gastrointestinal tract of the user. In some embodiments, the medicament compartment housing is flexible.

In some embodiments, an exterior contour of at least a portion of the housing is adapted to match an exterior contour of the medicament tablet. In some embodiments, the exterior contour of at least a portion of the housing is concave.

In some embodiments, the at least one biasing mechanism includes at least one pre-loaded compression spring. In some embodiments, the at least one biasing mechanism includes at least one flexible and resilient leaf.

In some embodiments, the device further includes the medicament tablet including the ingestible medicament.

In some embodiments, the medicament tablet has a diameter of up to 5 mm, up to 6 mm, up to 7 mm, up to 8 mm, or up to 9 mm.

In some embodiments, the medicament tablet has a maximal dimension of up to 10 mm.

In some embodiments, the medicament tablet has a volume of up to 100 mm³, up to 150 mm³, up to 200 mm³, up to 250 mm³, or up to 300 mm³.

In some embodiments, the medicament tablet has a height of up to 3 mm, up to 4 mm, or up to 5 mm.

In some embodiments, the ingestible medicament of the medicament tablet is absorbable in the stomach of the user. In some embodiments, the ingestible medicament of the medicament tablet is absorbable in the small intestine of the user.

In some embodiments, the ingestible medicament of the medicament tablet is suitable for treatment of one or more symptom or disease, selected from the group consisting of: Parkinsonism; Parkinson's Disease; progressive supranuclear palsy; corticobasal degeneration; multiple system atrophy; Parkinson-plus syndromes; any neurodegenerative disease in which the subject exhibits at least one, at least two, or at least three of the classical features of Parkinson's disease: tremor, postural instability, and akinesia or bradykesia; any neurodegenerative disease in which the subject positively responds to a dopaminergic treatment; any neurodegenerative disease in which the particular subject positively responds to an anticholinergic treatment; Constipation; Crohn's disease; Gastroparesis; irritable bowel syndrome (IBS); diarrhea or loose bowel movements; colitis; Hirschsprung's disease; Dyspepsia; and dysphagia.

In some embodiments, the ingestible medicament of the medicament tablet includes or includes an ingestible medicament selected from the group consisting of: levodopa; at least one dopaminergic agent; at least one catecholamine precursor; a dopamine precursor; at least one dopamine precursor agent; (L)-3,4-dihydroxyphenylalanine; N-methyl-N-(2-propynyl)-2-methyl-1-phenylethyl-2-amine; tyrosine hydroxylase; apomorphine; at least one anticholinergic agent; at least one agent selected to antagonize at least one cholinergic receptor; benzhexol; orphenadrine; at least one selective allosteric potentiator of metabotropic glutamate receptor 4 (mGluR4); N-phenyl-7-(hydroxylimino)cyclopropa[b] chromen-1a-carboxamide; at least one osmotic agent; magnesium citrate; magnesium hydroxide; polyethylene glycol; sodium phosphate; MiraLAX®; Polyethylene Glycol 3350; at least one contraction stimulating agent; bisacodyl; senna; Correctol®; Ducodyl; Dulcolax®; Senexon®; docusate sodium; Senokot®; senna glycoside; at least one stool softening agent; docusate sodium; Colace®; Linaclotide; Lactulose; lubiprostone; plecanatide; prucalopride; loperamide; and bismuth subsalicylate.

In accordance with an embodiment of the present invention, there is provided a method of delivering an ingestible medicament into a gastrointestinal tract of a user, the method including:
providing to the user the device as described hereinabove, for ingestion by the user;
following the user ingesting the device, controlling the vibrating ingestible capsule such that the vibration mode of operation at least partially transpires within at least one of an estimated absorption time period and an actual absorption time period of the ingestible medicament within the gastrointestinal tract of the user.

In accordance with an embodiment of the present invention, there is provided a method of delivering an ingestible medicament into a gastrointestinal tract of a user, the method including:
providing to the user the device as described herein;
inserting into the hollow of the medicament compartment housing the medicament tablet; and
following the user ingesting the device having the medicament tablet disposed in the hollow, controlling the vibrating ingestible capsule such that the vibration mode of operation at least partially transpires within at least one of an estimated absorption time period and an actual absorption time period of the ingestible medicament within the gastrointestinal tract of the user.

In accordance with an embodiment of the present invention, there is provided a hollow medicament delivery compartment adapted to be associated with a vibrating ingestible capsule for delivery of an ingestible medicament of a medicament tablet into the gastrointestinal tract of a user, the hollow medicament delivery compartment being dimensioned and configured to have the medicament tablet disposed therein, and including at least one aperture formed in the hollow medicament delivery compartment,
wherein the at least one aperture is dimensioned and configured to enable fluid communication between an environment surrounding the hollow medicament delivery compartment.

In some embodiments, the at least one aperture is dimensioned and configured such that, when the medicament tablet is disposed within the hollow and the hollow medicament delivery compartment is in the gastrointestinal tract of the user, the ingestible medicament of the medicament tablet enters the environment surrounding the hollow medicament delivery compartment for delivery thereof to the body of the user. In some embodiments, the hollow medicament delivery compartment further includes at least one biasing mechanism adapted, when the medicament tablet is disposed within the hollow of the hollow medicament delivery compartment, to bias the medicament tablet toward the vibrating ingestible capsule.

In some embodiments, the at least one biasing mechanism includes at least one longitudinal biasing mechanism adapted to bias the medicament tablet by application of pressure along a longitudinal axis of the medicament tablet. In some embodiments, the at least one biasing mechanism includes at least one radial biasing mechanism adapted to bias the medicament tablet by application of radial pressure along a perimeter of the medicament tablet.

In some embodiments, the hollow medicament delivery compartment includes an attachment mechanism for mutual attachment to a corresponding attachment mechanism of the vibrating ingestible capsule.

In some embodiments, the hollow medicament delivery compartment is adapted to be at least partially attached to the vibrating ingestible capsule. In some embodiments, the hollow medicament delivery compartment is adapted to be attached to the vibrating ingestible capsule. In some embodiments, the hollow medicament delivery compartment is adapted to be fixedly attached to the vibrating ingestible capsule.

In some embodiments, the hollow medicament delivery compartment is adapted to be attached to the vibrating ingestible capsule by snap fit engagement. In some embodiments, the hollow medicament delivery compartment is adapted to be attached to the vibrating ingestible capsule by threaded engagement. In some embodiments, the hollow medicament delivery compartment is adapted to be attached to the vibrating ingestible capsule by adhering. In some embodiments, the hollow medicament delivery compartment is adapted to be attached to the vibrating ingestible capsule by soldering.

In some embodiments, the hollow medicament delivery compartment is adapted to at least partially envelop the vibrating ingestible capsule. In some embodiments, the hollow medicament delivery compartment is adapted to fully envelop the vibrating ingestible capsule. In some embodiments, the hollow medicament delivery compartment includes a hollow capsule including the at least one aperture, having the vibrating ingestible capsule disposed therein.

In some embodiments, the hollow medicament delivery compartment has a volume in the range of 200 mm³ to 800 mm³, 300 mm³ to 700 mm³, 400 mm³ to 600 mm³, or 500 mm³.

In some embodiments, the at least one aperture is dimensioned and configured such that when the hollow medicament delivery compartment is attached to the vibrating ingestible capsule, the medicament tablet, while whole, cannot be removed from the hollow medicament delivery compartment.

In some embodiments, the at least one aperture is dimensioned and configured to enable insertion of the medicament tablet, via the at least one aperture, into the hollow medicament delivery compartment.

In some embodiments, in use within the gastrointestinal tract of the user, the biasing mechanism is adapted to continue biasing the medicament tablet toward the ingestible vibrating capsule, while the ingestible medicament from the medicament tablet is delivered to the environment surrounding the hollow medicament delivery compartment.

In some embodiments, the hollow medicament delivery compartment is biodegradable. In some embodiments, the hollow medicament delivery compartment is digestible by the gastrointestinal tract of the user. In some embodiments, the hollow medicament delivery compartment is flexible.

In some embodiments, the at least one biasing mechanism includes at least one pre-loaded compression spring. In some embodiments, the at least one biasing mechanism includes at least one flexible and resilient leaf.

In some embodiments, the hollow medicament delivery compartment further includes the medicament tablet including the medicament disposed within the hollow medicament delivery compartment.

In some embodiments, the medicament tablet has a diameter of up to 5 mm, up to 6 mm, up to 7 mm, up to 8 mm, or up to 9 mm.

In some embodiments, the medicament tablet has a maximal dimension of up to 10 mm.

In some embodiments, the medicament tablet has a volume of 100 mm³, up to 150 mm³, up to 200 mm³, up to 250 mm³, or up to 300 mm³.

In some embodiments, the medicament tablet has a height of up to 3 mm, up to 4 mm, or up to 5 mm.

In some embodiments, the ingestible medicament of the medicament tablet is absorbable in the stomach of the user. In some embodiments, the ingestible medicament of the medicament tablet is absorbable in the small intestine of the user.

In some embodiments, the ingestible medicament of the medicament tablet is suitable for treatment of one or more symptom or disease, selected from the group consisting of: Parkinsonism; Parkinson's Disease; progressive supranuclear palsy; corticobasal degeneration; multiple system atrophy; Parkinson-plus syndromes; any neurodegenerative disease in which the subject exhibits at least one, at least two, or at least three of the classical features of Parkinson's disease: tremor, postural instability, and akinesia or bradykesia; any neurodegenerative disease in which the subject positively responds to a dopaminergic treatment; any neurodegenerative disease in which the particular subject positively responds to an anticholinergic treatment; Constipation; Crohn's disease; Gastroparesis; irritable bowel syndrome (IBS); diarrhea or loose bowel movements; colitis; Hirschsprung's disease; Dyspepsia; and dysphagia.

In some embodiments, the ingestible medicament of the medicament tablet includes or includes an ingestible medicament selected from the group consisting of: Levodopa; at least one dopaminergic agent; at least one catecholamine precursor; a dopamine precursor; at least one dopamine precursor agent; (L)-3,4-dihydroxyphenylalanine; N-methyl-N-(2-propynyl)-2-methyl-1-phenylethyl-2-amine; tyrosine hydroxylase; apomorphine; at least one anticholinergic agent; at least one agent selected to antagonize at least one cholinergic receptor; benzhexol; orphenadrine; at least one selective allosteric potentiator of metabotropic glutamate receptor 4 (mGluR4); N-phenyl-7-(hydroxylimino)cyclopropa[b] chromen-1a-carboxamide; at least one osmotic agent; magnesium citrate; magnesium hydroxide; polyethylene glycol; sodium phosphate; MiraLAX®; at least one contraction stimulating agent; bisacodyl; senna; Correctol; Ducodyl; Dulcolax; Senexon; Senokot; at least one stool softening agent; docusate sodium; Colace; Linaclotide; Lactulose; Lubiprostone; Plecanatide; Prucaltride; Loperamide; and bismuth subsalicylate.

In accordance with an embodiment of the present invention, there is provided a vibrating ingestible capsule adapted to be associated with a hollow medicament delivery compartment for delivering an ingestible medicament of a medicament tablet into the gastrointestinal tract of a user, the vibrating ingestible capsule including:
a housing including an attachment mechanism adapted for mutual attachment to a corresponding attachment mechanism of the hollow medicament delivery compartment;
a vibrating agitator disposed within the housing and adapted such that, in a vibration mode of operation, the housing exerts vibrations on an environment surrounding the vibrating gastrointestinal capsule;
a power supply disposed within the housing and adapted to power the vibrating agitator; and
a control element adapted to activate the vibrating agitator to be operative in the vibration mode of operation,
wherein at least one vibration parameter of the vibrating agitator is set so as to promote absorption of the ingestible medicament into the bloodstream of the user.

In some embodiments, the at least one vibration parameter includes at least one of a vibration frequency, a cumulative vibration duration, a number of vibration cycles per time unit, a duration of a vibration duration within a vibration cycle, a duration of a repose duration within a vibration cycle, a total duration of a single vibration cycle, and a net force exerted by the housing on the environment.

In some embodiments, the control element is adapted to control a timing or activation delay of the vibration mode of operation such that the vibration mode of operation at least partially transpires within at least one of an estimated absorption time period and an actual absorption time period of medicament released from the medicament tablet within the gastrointestinal tract of the user.

In some embodiments, the control element is adapted to activate the vibrating agitator to be operative in the vibration mode of operation in response to receipt of an activation input.

In some embodiments, the vibrating ingestible capsule further includes at least one sensor adapted to provide the activation input to the control element. In some embodiments, the at least one sensor forms part of the vibrating ingestible capsule.

In some embodiments, the at least one sensor includes an illumination sensor, adapted to provide an input indicating transition of the vibrating ingestible capsule from an illuminated environment to a dark environment.

In some embodiments, the at least one sensor includes a pressure sensor, adapted to provide an input indicating pressure applied to the vibrating ingestible capsule, which pressure is indicative of the vibrating ingestible capsule moving through a pharynx of the user.

In some embodiments, the at least one sensor includes a temperature sensor, adapted to provide an input indicating transition of the vibrating ingestible capsule from an area with ambient temperature to an area with a human body temperature.

In some embodiments, the at least one sensor includes an accelerometer, adapted to provide an input in response to a detected activation motion carried out with the vibrating ingestible capsule.

In some embodiments, the at least one sensor includes a moisture sensor, adapted to provide an input indicating transition of the vibrating ingestible capsule from a dry environment to a humid environment.

In some embodiments, the vibrating ingestible capsule is functionally associated with a control unit remote from the vibrating ingestible capsule, and wherein the control element is adapted to receive the activation input from the control unit.

In some embodiments, the control element is adapted to receive the activation input following ingesting of the vibrating ingestible capsule.

In some embodiments, the control element is adapted to receive the activation input prior to ingesting of the vibrating ingestible capsule.

In some embodiments, the control element is adapted to receive the activation input by receiving a vibration protocol to be used by the control element to control operation of the vibrating agitator.

In some embodiments, the vibrating agitator includes at least a radial agitator adapted, in the vibration mode of operation, to exert radial forces on the housing, in a radial direction with respect to a or the longitudinal axis of the housing, thereby to cause the vibrations exerted by the housing.

In some embodiments, the vibrating agitator includes at least an axial agitator adapted, in the vibration mode of operation, to exert axial forces on the housing, in an axial direction with respect to a or the longitudinal axis of the housing, thereby to cause the vibrations exerted by the housing.

In some embodiments, the vibrating agitator includes a radial agitator adapted, in the vibration mode of operation, to exert radial forces on the housing, in a radial direction with respect to a or the longitudinal axis of the housing, and a separate axial agitator adapted, in the vibration mode of operation, to exert axial forces on the housing, in an axial direction with respect to a or the longitudinal axis of the housing.

In some embodiments, the vibrating agitator includes a single agitator adapted, in the vibration mode of operation, to exert radial forces on the housing in a radial direction with respect to a or the longitudinal axis of the housing, and to exert axial forces on the housing, in an axial direction with respect to the longitudinal axis of the housing.

In some embodiments, the control element is adapted to control the vibrating agitator such that the vibrating mode of operation includes a plurality of cycles, each of the cycles including a vibration duration followed by a repose duration, wherein the housing exerts the vibrations during the vibration duration.

In some embodiments, the repose duration is greater than the vibration duration. In some embodiments, the vibration duration is in the range of 0.1 second to 10 seconds, 1 second to 10 seconds, 1 second to 9 seconds, 2 seconds to 9 seconds, 3 seconds to 9 seconds, 3 seconds to 8 seconds, 3 seconds to 7 seconds, 3 seconds to 6 seconds, 4 seconds to 6 seconds, or 5 seconds to 6 seconds.

In some embodiments, the repose duration is in the range of 1 second to 180 seconds, 3 seconds to 180 seconds, 5 seconds to 180 seconds, 5 seconds to 150 seconds, seconds to 120 seconds, 8 seconds to 100 seconds, 8 seconds to 30 seconds, 10 seconds to 80 seconds, 10 seconds to 70 seconds, 10 seconds to 60 seconds, 10 seconds to 50 seconds, 10 seconds to 40 seconds, 10 seconds to 30 seconds, 10 seconds to 20 seconds, or 15 seconds to 20 seconds.

In some embodiments, a duration of each of the plurality of cycles is in the range of 1.1 seconds to 200 seconds, 5 seconds to 200 seconds, 10 seconds to 200 seconds, 10 seconds to 150 seconds, 10 seconds to 100 seconds, 10 seconds to 80 seconds, 10 seconds to 50 seconds, 10 seconds to 40 seconds, 10 seconds to 30 seconds, 15 seconds to 50 seconds, 15 seconds to 40 seconds, 15 seconds to 30 seconds, or 15 seconds to 25 seconds.

In some embodiments, the control element is adapted to control the vibrating agitator such that a cumulative duration of the vibrating mode of operation is in the range of 1 hour to 12 hours, 2 hours to 10 hours, 2 hours to 8 hours, 2 hours to 6 hours, 2 hours to 4 hours, or 2 hours to 3 hours.

In some embodiments, the vibrating agitator is configured to exert forces on the housing of the vibrating ingestible capsule, such that a net force exerted by the housing on the environment thereof is in the range of 50 grams force (gf) to 600 gf, 50 gf to 550 gf, 100 gf to 550 gf, 100 gf to 500 gf, 150 gf to 500 gf, 200 gf to 500 gf, or 200 gf to 450 gf. In some embodiments, the vibrating agitator is configured to exert the forces on the housing to attain a housing vibrational frequency within a range of 10 Hz to 650 Hz, 15 Hz to 600 Hz, 20 Hz to 550 Hz, 30 Hz to 550 Hz, 50 Hz to 500 Hz, 70 Hz to 500 Hz, 100 Hz to 500 Hz, 130 Hz to 500 Hz, or 150 Hz to 500 Hz.

In some embodiments, the housing is adapted to be at least partially attached to the hollow medicament delivery compartment. In some embodiments, the housing is adapted to be attached to the hollow medicament delivery compartment. In some embodiments, the housing is adapted to be fixedly attached to the hollow medicament delivery compartment.

In some embodiments, the housing is adapted to be attached to the hollow medicament delivery compartment by snap fit engagement. In some embodiments, the housing is adapted to be attached to the hollow medicament delivery compartment by threaded engagement. In some embodiments, the housing is adapted to be attached to the hollow medicament delivery compartment by adhering. In some embodiments, the housing is adapted to be attached to the hollow medicament delivery compartment by soldering.

In some embodiments, the housing is adapted to be at least partially enveloped by the hollow medicament delivery compartment. In some embodiments, the housing is adapted to be fully enveloped by the hollow medicament delivery compartment.

In accordance with an embodiment of the present invention, there is provided a method for delivering an ingestible medicament of a medicament tablet into the gastrointestinal tract of a user, the method including:
providing a vibrating ingestible capsule including:
   a housing;
   a vibrating agitator disposed within the housing and adapted such that, in a vibration mode of operation, the housing exerts vibrations on an environment surrounding the vibrating gastrointestinal capsule;
   a power supply disposed within the housing and adapted to power the vibrating agitator; and
   a control element adapted to activate the vibrating agitator to be operative in the vibration mode of operation; and
associating a hollow medicament compartment housing with the housing, the hollow medicament compartment having at least one aperture formed therein;
inserting the medicament tablet into the hollow medicament compartment housing;
ingesting an assembly of the vibrating ingestible capsule, the hollow medicament compartment housing, and the medicament tablet by the user; and
following the user ingesting the assembly, controlling the vibrating ingestible capsule such that the vibration mode of operation at least partially transpires within at least one of an estimated absorption time period and an actual absorption time period of the ingestible medicament within the gastrointestinal tract of the user.

In some embodiments, controlling the vibrating ingestible capsule includes setting at least one vibration parameter of the vibrating ingestible capsule so as to promote absorption of the ingestible medicament into the bloodstream of the user. In some embodiments, setting the at least one vibration parameter includes setting at least one of a vibration frequency, a cumulative vibration duration, a number of vibration cycles per time unit, a duration of a vibration duration within a vibration cycle, a duration of a repose duration within a vibration cycle, a total duration of a single vibration cycle, and a net force exerted by the housing on the environment.

In some embodiments, the method further includes, following the inserting the medicament tablet, biasing the medicament tablet toward the housing of the vibrating ingestible capsule. In some embodiments, biasing includes biasing the medicament tablet by application of pressure along a longitudinal axis of the medicament tablet. In some embodiments, biasing includes biasing the medicament tablet by application of radial pressure along a perimeter of the medicament tablet.

In some embodiments, the housing of the vibrating ingestible capsule includes an attachment mechanism, the hollow medicament compartment housing includes a corresponding attachment mechanism, and the associating includes mutually attaching the vibrating ingestible capsule to the hollow medicament compartment housing.

In some embodiments, controlling includes controlling a timing or activation delay of the vibration mode of operation such that the vibration mode of operation at least partially transpires within at least one of an estimated absorption time period and an actual absorption time period of an ingestible medicament released from the medicament tablet within the gastrointestinal tract of the user.

In some embodiments, the method further including, prior to the controlling, at the control element receiving an activation input, and the controlling includes activating the vibrating agitator to be operative in the vibration mode of operation following the receiving the activation input.

In some embodiments, receiving the activation input includes receiving the activation input from at least one sensor.

In some embodiments, receiving the activation input includes receiving, from an illumination sensor, an input indicating transition of the assembly from an illuminated environment to a dark environment.

In some embodiments, receiving the activation input includes receiving, from a pressure sensor, an input indicating pressure applied to the assembly, which pressure is indicative of the assembly moving through a pharynx of the user.

In some embodiments, receiving the activation input includes receiving, from a temperature sensor, an input indicating transition of the assembly from an area with ambient temperature to an area with a human body temperature.

In some embodiments, receiving the activation input includes receiving, from an accelerometer, an input in response to a detected activation motion carried out with the assembly.

In some embodiments, receiving the activation input includes receiving, from a moisture sensor, an input indicating transition of the assembly from a dry environment to a humid environment.

In some embodiments, receiving the activation input includes receiving the activation input from a control unit remote from the assembly and functionally associated with the control element.

In some embodiments, receiving the activation input occurs following the ingesting of the assembly.

In some embodiments, receiving the activation input occurs prior to the ingesting of the assembly.

In some embodiments, receiving the activation input includes receiving a vibration protocol to be used by the control element for the controlling operation of the vibrating agitator.

In some embodiments, controlling the vibrating agitator includes controlling the vibrating agitator such that the vibrating mode of operation includes a plurality of cycles, each of the cycles including a vibration duration followed by a repose duration, wherein the housing exerts the vibrations during the vibration duration.

In some embodiments, the repose duration is greater than the vibration duration.

In some embodiments, the vibration duration is in the range of 0.1 second to 10 seconds, 1 second to 10 seconds, 1 second to 9 seconds, 2 seconds to 9 seconds, 3 seconds to 9 seconds, 3 seconds to 8 seconds, 3 seconds to 7 seconds, 3 seconds to 6 seconds, 4 seconds to 6 seconds, or 5 seconds to 6 seconds.

In some embodiments, the repose duration is in the range of 1 second to 180 seconds, 3 seconds to 180 seconds, 5 seconds to 180 seconds, 5 seconds to 150 seconds, seconds to 120 seconds, 8 seconds to 100 seconds, 8 seconds to 30 seconds, 10 seconds to 80 seconds, 10 seconds to 70 seconds, 10 seconds to 60 seconds, 10 seconds to 50 seconds, 10 seconds to 40 seconds, 10 seconds to 30 seconds, 10 seconds to 20 seconds, or 15 seconds to 20 seconds.

In some embodiments, a duration of each of the plurality of cycles is in the range of 1.1 seconds to 200 seconds, 5 seconds to 200 seconds, 10 seconds to 200 seconds, 10 seconds to 150 seconds, 10 seconds to 100 seconds, 10 seconds to 80 seconds, 10 seconds to 50 seconds, 10 seconds to 40 seconds, 10 seconds to 30 seconds, 15 seconds to 50 seconds, 15 seconds to 40 seconds, 15 seconds to 30 seconds, or 15 seconds to 25 seconds.

In some embodiments, controlling includes controlling the vibrating agitator such that a cumulative duration of the vibrating mode of operation is in the range of 1 hour to 12 hours, 2 hours to 10 hours, 2 hours to 8 hours, 2 hours to 6 hours, 2 hours to 4 hours, or 2 hours to 3 hours.

In some embodiments, controlling includes controlling the vibrating agitator to exert forces on the housing of the vibrating ingestible capsule, such that a net force exerted by the housing on the environment thereof is in the range of 50 grams force (gf) to 600 gf, 50 gf to 550 gf, 100 gf to 550 gf, 100 gf to 500 gf, 150 gf to 500 gf, 200 gf to 500 gf, or 200 gf to 450 gf.

In some embodiments, controlling includes controlling the vibrating agitator to exert the forces on the housing to attain a housing vibrational frequency within a range of 10 Hz to 650 Hz, 15 Hz to 600 Hz, 20 Hz to 550 Hz, 30 Hz to 550 Hz, 50 Hz to 500 Hz, 70 Hz to 500 Hz, 100 Hz to 500 Hz, 130 Hz to 500 Hz, or 150 Hz to 500 Hz.

In some embodiments, associating includes at least partially attaching the medicament compartment housing to the vibrating ingestible capsule.

In some embodiments, associating includes attaching the medicament compartment housing to the vibrating ingestible capsule. In some embodiments, associating includes fixedly attaching the medicament compartment housing to the vibrating ingestible capsule.

In some embodiments, associating includes attaching the medicament compartment housing to the vibrating ingestible capsule by snap fit engagement. In some embodiments, associating includes attaching the medicament compartment housing to the vibrating ingestible capsule by threaded engagement. In some embodiments, associating includes attaching the medicament compartment housing to the vibrating ingestible capsule by adhering. In some embodiments, associating includes attaching the medicament compartment housing to the vibrating ingestible capsule by soldering.

In some embodiments, associating includes at least partially enveloping the medicament compartment housing around the vibrating ingestible capsule. In some embodiments, associating includes fully enveloping the medicament compartment housing around the vibrating ingestible capsule.

In some embodiments, inserting includes inserting the medicament tablet, via the at least one aperture, into the hollow medicament compartment housing.

In some embodiments, the medicament tablet has a diameter of up to 5 mm, up to 6 mm, up to 7 mm, up to 8 mm, or up to 9 mm.

In some embodiments, the medicament tablet has a maximal dimension of up to 10 mm.

In some embodiments, the medicament tablet has a volume of up to 100 mm³, up to 150 mm³, up to 200 mm³, up to 250 mm³, or up to 300 mm³.

In some embodiments, the medicament tablet has a height of up to 3 mm, up to 4 mm, or up to 5 mm.

In some embodiments, the ingestible medicament of the medicament tablet is absorbable in the stomach of the user. In some embodiments, the medicament tablet includes an ingestible medicament absorbable in the small intestine of the user.

In some embodiments, the ingestible medicament of the medicament tablet is suitable for treatment of one or more symptom or disease, selected from the group consisting of: Parkinsonism; Parkinson's Disease; progressive supranuclear palsy; corticobasal degeneration; multiple system atrophy; Parkinson-plus syndromes; any neurodegenerative disease in which the subject exhibits at least one, at least two, or at least three of the classical features of Parkinson's disease: tremor, postural instability, and akinesia or bradykesia; any neurodegenerative disease in which the subject positively responds to a dopaminergic treatment; any neurodegenerative disease in which the particular subject positively responds to an anticholinergic treatment; Constipation; Crohn's disease; Gastroparesis; irritable bowel syndrome (IBS); diarrhea or loose bowel movements; colitis; Hirschsprung's disease; Dyspepsia; and dysphagia.

In some embodiments, the ingestible medicament of the medicament tablet includes or includes an ingestible medicament selected from the group consisting of: Levodopa; at least one dopaminergic agent; at least one catecholamine precursor; a dopamine precursor; at least one dopamine precursor agent; (L)-3,4-dihydroxyphenylalanine; N-methyl-N-(2-propynyl)-2-methyl-1-phenylethyl-2-amine; tyrosine hydroxylase; apomorphine; at least one anticholinergic agent; at least one agent selected to antagonize at least one cholinergic receptor; benzhexol; orphenadrine; at least one selective allosteric potentiator of metabotropic glutamate receptor 4 (mGluR4); N-phenyl-7-(hydroxylimino)cyclopropa[b] chromen-1a-carboxamide; at least one osmotic agent; magnesium citrate; magnesium hydroxide; polyethylene glycol; sodium phosphate; MiraLAX®; at least one contraction stimulating agent; bisacodyl; senna; Correctol; Ducodyl; Dulcolax; Senexon; Senokot; at least one stool softening agent; docusate sodium; Colace; Linaclotide; Lactulose; Lubiprostone; Plecanatide; Prucaltride; Loperamide; and bismuth subsalicylate.

In accordance with an embodiment of the present invention, there is provided a device for delivering a flowable ingestible medicament into the gastrointestinal tract of a user, the device including:
a housing including a first housing portion and a second housing portion, the second housing portion having a portal formed therein;
a vibrating agitator disposed within the housing and adapted such that, in a vibration mode of operation, the housing exerts vibrations on an environment surrounding the vibrating ingestible capsule;
a power supply disposed within the housing and adapted to power the vibrating agitator; and a control element adapted to activate the vibrating agitator to be operative in the vibration mode of operation;
a flexible and collapsible medicament reservoir dimensioned to contain the flowable ingestible medicament;
a reservoir biasing mechanism adapted to apply pressure to the flexible and collapsible medicament reservoir;
a conduit extending from the medicament reservoir to the portal, and sealing the portal; and
a valve including a weight and a valve biasing mechanism adapted, in a closed operative orientation, to bias the weight against the conduit so as to block flow through the conduit, and in an open operative orientation to remove the weight from the conduit so as to allow fluid to flow through the conduit,
wherein the valve is functionally associated with at least one of the housing and the vibrating agitator, such that when the vibrating agitator is in the vibration mode of operation, at least some of the vibrations exerted by the vibrating agitator or by the housing are applied to the valve biasing mechanism and periodically transition the valve between the closed operative orientation and the open operative orientation.

In some embodiments, when the vibrating agitator is in the vibration mode of operation and the valve is in the open operative orientation, pressure applied by the reservoir biasing mechanism to the reservoir causes the flowable ingestible medicament to flow from the reservoir, via the conduit and the portal, to an environment surrounding the device.

In some embodiments, at least one vibration parameter of the vibrating agitator is set so as to promote transitioning of the valve between the closed operative orientation and the open operative orientation. In some embodiments, at least one vibration parameter of the vibrating agitator is set so as to promote absorption of the ingestible medicament into the bloodstream of the user.

In some embodiments, at least one valve parameter of the valve is set such that the valve functions as a gear reducer, opening and closing at a frequency smaller than a frequency of the vibrating agitator. In some embodiments, the at least one valve parameter includes a mass of the weight, a length of the valve biasing mechanism, and a spring constant of the valve biasing mechanism.

In some embodiments, the at least one vibration parameter includes at least one of a vibration frequency, a cumulative vibration duration, a number of vibration cycles per time unit, a duration of a vibration duration within a vibration cycle, a duration of a repose duration within a vibration cycle, a total duration of a single vibration cycle, and a net force exerted by the housing on the environment.

In some embodiments, the first housing portion is a sealed housing portion accommodating the vibrating agitator, the power supply, and the control element, the second housing portion accommodates the flexible and collapsible medicament reservoir, the reservoir biasing mechanism, the conduit, and the valve, the first housing portion and the second housing portion are separate elements, which are attached to each other to form the housing, and the valve is functionally associated with the first housing portion such that when the vibrating agitator is in the vibration mode of operation, at least some of the vibrations exerted by the first housing portion are applied to the valve biasing mechanism and periodically transition the valve between the closed operative orientation and the open operative orientation.

In some embodiments, the second housing portion is fixedly attached to the first housing portion. In some embodiments, the second housing portion is attached to the first housing portion by snap fit engagement. In some embodiments, the second housing portion is attached to the first housing portion by threaded engagement. In some embodiments, the second housing portion is attached to the first housing portion by adhering. In some embodiments, the second housing portion is attached to the first housing portion by soldering.

In some embodiments, the first and second housing portions form a single housing element including the portal and defining a single hollow, the single hollow accommodating the vibrating agitator, the power supply, the control element, the flexible and collapsible medicament reservoir, the reservoir biasing mechanism, and the flexible and resilient conduit.

In some embodiments, a hollow of the second housing portion has a volume in the range of 200 mm³ to 3000 mm³, 200 mm³ to 800 mm³, 100 mm³ to 600 mm³, 400 mm³ to 1000 mm³, 700 mm³ to 1500 mm³, or 1400 mm³ to 3000 mm³

In some embodiments, the flexible and collapsible medicament reservoir has a maximal volume in the range of 0.5 ml to 15 ml. In some embodiments, the flexible and collapsible medicament reservoir has a Young's modulus smaller than 1 GPa. In some embodiments, the flexible and collapsible medicament reservoir is formed of a material selected from the group consisting of: silicone rubber, natural rubber, Polyethylene, and PVC.

In some embodiments, the reservoir biasing mechanism includes a reservoir spring terminating in a pressure applying surface, the pressure applying surface engaging an exterior surface of the medicament reservoir. In some embodiments, the reservoir spring is anchored to the second housing portion. In some embodiments, the reservoir biasing mechanism has a spring constant in the range of 1 N/m to 200 N/m. In some embodiments, the conduit is integrally formed with the medicament reservoir. In some embodiments, the conduit is formed of a different material than the medicament reservoir. In some embodiments, a recovery time of the conduit is at most 0.1 seconds.

In some embodiments, the vibrating agitator has a frequency f, and a recovery time of the conduit is at most equal to 1/f. In some embodiments, the valve biasing mechanism has a frequency fv of moving from the open operative orientation to the closed operative orientation and back to the open operative orientation, and a recovery time of the conduit is at most equal to 1/fv.

In some embodiments, the conduit has a diameter in the range of 0.01 mm to 0.9 mm. In some embodiments, the conduit has a length in the range of 3 mm to 25 mm. In some embodiments, the conduit is at least one of, and optionally both of, flexible and resilient.

In some embodiments, the valve biasing mechanism is anchored to the first housing portion. In some embodiments, the valve biasing mechanism is anchored to the second housing portion. In some embodiments, the valve biasing mechanism has a spring constant in the range of 0.1 N/m to 5 N/m.

In some embodiments, the weight has a mass in the range of 0.1 grams to 2 grams. In some embodiments, the weight includes the vibrating agitator.

In some embodiments, the vibrations exerted by the vibrating agitator are transferred, through an anchoring point of the valve biasing mechanism, to the valve biasing mechanism, thereby causing rocking of the valve biasing mechanism resulting in the periodic transitioning of the valve between the closed operative orientation and the open operative orientation.

In some embodiments, the control element is adapted to control a timing or activation delay of the vibration mode of operation such that the vibration mode of operation at least partially transpires within a region of the gastrointestinal tract in which the flowable ingestible medicament is absorbable by the body of the user.

In some embodiments, the control element is adapted to control a timing or activation delay of the vibrating mode of operation such that the vibration mode of operation at least partially transpires during at least one of an estimated absorption time period and an actual absorption time period of the flowable ingestible medicament released from the reservoir, via the conduit and the portal, into the gastrointestinal tract of the user.

In some embodiments, the control element is adapted to activate the vibrating agitator to be operative in the vibration mode of operation in response to receipt of an activation input.

In some embodiments, the device further includes at least one sensor adapted to provide the activation input to the control element. In some embodiments, the at least one sensor forms part of the vibrating ingestible capsule.

In some embodiments, the at least one sensor includes an illumination sensor, adapted to provide an input indicating transition of the device from an illuminated environment to a dark environment.

In some embodiments, the at least one sensor includes a pressure sensor, adapted to provide an input indicating pressure applied to the device, which pressure is indicative of the device moving through a pharynx of the user.

In some embodiments, the at least one sensor includes a temperature sensor, adapted to provide an input indicating transition of the device from an area with ambient temperature to an area with a human body temperature.

In some embodiments, the at least one sensor includes an accelerometer, adapted to provide an input in response to a detected activation motion carried out with the device.

In some embodiments, the at least one sensor includes a moisture sensor, adapted to provide an input indicating transition of the device from a dry environment to a humid environment.

In some embodiments, the device is functionally associated with a control unit remote from the device, and the control element is adapted to receive the activation input from the control unit.

In some embodiments, the control element is adapted to receive the activation input following ingesting of the device.

In some embodiments, the control element is adapted to receive the activation input prior to ingesting of the device.

In some embodiments, the control element is adapted to receive the activation input by receiving a vibration protocol to be used by the control element to control operation of the vibrating agitator.

In some embodiments, the vibrating agitator includes at least a radial agitation mechanism adapted, in the vibration mode of operation, to exert radial forces on the housing, in a radial direction with respect to a or the longitudinal axis of the housing, thereby to cause the vibrations exerted by the housing.

In some embodiments, the vibrating agitator includes at least an axial agitation mechanism adapted, in the vibration mode of operation, to exert axial forces on the housing, in an axial direction with respect to a or the longitudinal axis of the housing, thereby to cause the vibrations exerted by the housing.

In some embodiments, the vibrating agitator includes a radial agitation mechanism adapted, in the vibration mode of operation, to exert radial forces on the housing, in a radial direction with respect to a or the longitudinal axis of the housing, and a separate axial agitation mechanism adapted, in the vibration mode of operation, to exert axial forces on the housing, in an axial direction with respect to a or the longitudinal axis of the housing.

In some embodiments, the vibrating agitator includes a single agitation mechanism adapted, in the vibration mode of operation, to exert radial forces on the housing in a radial direction with respect to a or the longitudinal axis of the housing, and to exert axial forces on the housing, in an axial direction with respect to the longitudinal axis of the housing.

In some embodiments, the control element is adapted to control the vibrating agitator such that the vibrating mode of operation includes a plurality of cycles, each of the cycles including a vibration duration followed by a repose duration, wherein the housing exerts the vibrations during the vibration duration. In some embodiments, the repose duration is greater than the vibration duration.

In some embodiments, the vibration duration is in the range of 0.1 second to 10 seconds, 1 second to 10 seconds, 1 second to 9 seconds, 2 seconds to 9 seconds, 3 seconds to 9 seconds, 3 seconds to 8 seconds, 3 seconds to 7 seconds, 3 seconds to 6 seconds, 4 seconds to 6 seconds, or 5 seconds to 6 seconds.

In some embodiments, the repose duration is in the range of 1 second to 180 seconds, 3 seconds to 180 seconds, 5 seconds to 180 seconds, 5 seconds to 150 seconds, seconds to 120 seconds, 8 seconds to 100 seconds, 8 seconds to 30 seconds, 10 seconds to 80 seconds, 10 seconds to 70 seconds, 10 seconds to 60 seconds, 10 seconds to 50 seconds, 10 seconds to 40 seconds, 10 seconds to 30 seconds, 10 seconds to 20 seconds, or 15 seconds to 20 seconds.

In some embodiments, a duration of each of the plurality of cycles is in the range of 1.1 seconds to 200 seconds, 5 seconds to 200 seconds, 10 seconds to 200 seconds, 10 seconds to 150 seconds, 10 seconds to 100 seconds, 10 seconds to 80 seconds, 10 seconds to 50 seconds, 10 seconds to 40 seconds, 10 seconds to 30 seconds, 15 seconds to 50 seconds, seconds to 40 seconds, 15 seconds to 30 seconds, or 15 seconds to 25 seconds.

In some embodiments, the control element is adapted to control the vibrating agitator such that a cumulative duration of the vibrating mode of operation is in the range of 1 hour to 12 hours, 2 hours to 10 hours, 2 hours to 8 hours, 2 hours to 6 hours, 2 hours to 4 hours, or 2 hours to 3 hours.

In some embodiments, the vibrating agitator is configured to exert forces on the housing of the vibrating ingestible capsule, such that a net force exerted by the housing on the environment thereof is in the range of 50 grams force (gf) to 600 gf, 50 gf to 550 gf, 100 gf to 550 gf, 100 gf to 500 gf, 150 gf to 500 gf, 200 gf to 500 gf, or 200 gf to 450 gf.

In some embodiments, the vibrating agitator is configured to exert the forces on the housing to attain a housing vibrational frequency within a range of 10 Hz to 650 Hz, 15 Hz to 600 Hz, 20 Hz to 550 Hz, 30 Hz to 550 Hz, 50 Hz to 500 Hz, 70 Hz to 500 Hz, 100 Hz to 500 Hz, 130 Hz to 500 Hz, or 150 Hz to 500 Hz.

In some embodiments, the device further includes the flowable ingestible medicament disposed within the medicament reservoir.

In some embodiments, the flowable ingestible medicament has a viscosity in the range of 100 Pa·s to 1000 Pa·s.

In some embodiments, the flowable ingestible medicament is absorbable in the stomach of the user. In some embodiments, the flowable ingestible medicament is absorbable in the small intestine of the user.

In some embodiments, the flowable ingestible medicament is suitable for treatment of one or more symptom or disease, selected from the group consisting of: Parkinsonism; Parkinson's Disease; progressive supranuclear palsy; corticobasal degeneration; multiple system atrophy; Parkinson-plus syndromes; any neurodegenerative disease in which the subject exhibits at least one, at least two, or at least three of the classical features of Parkinson's disease: tremor, postural instability, and akinesia or bradykesia; any neurodegenerative disease in which the subject positively responds to a dopaminergic treatment; any neurodegenerative disease in which the particular subject positively responds to an anticholinergic treatment; Constipation; Crohn's disease; Gastroparesis; irritable bowel syndrome (IBS); diarrhea or loose bowel movements; colitis; Hirschsprung's disease; Dyspepsia; and dysphagia.

In some embodiments, the flowable ingestible medicament includes or includes an ingestible medicament selected from the group consisting of: Levodopa; at least one dopaminergic agent; at least one catecholamine precursor; a dopamine precursor; at least one dopamine precursor agent; (L)-3,4-dihydroxyphenylalanine; N-methyl-N-(2-propynyl)-2-methyl-1-phenylethyl-2-amine; tyrosine hydroxylase; apomorphine; at least one anticholinergic agent; at least one agent selected to antagonize at least one cholinergic receptor; benzhexol; orphenadrine; at least one selective allosteric potentiator of metabotropic glutamate receptor 4 (mGluR4); N-phenyl-7-(hydroxylimino)cyclopropa[b]chromen-1a-carboxamide; at least one osmotic agent; magnesium citrate; magnesium hydroxide; polyethylene glycol; sodium phosphate; MiraLAX®; at least one contraction stimulating agent; bisacodyl; senna; Correctol; Ducodyl; Dulcolax; Senexon; Senokot; at least one stool softening agent; docusate sodium; Colace; Linaclotide; Lactulose; Lubiprostone; Plecanatide; Prucaltride; Loperamide; and bismuth subsalicylate.

In accordance with another embodiment of the present invention, there is provided a method of delivering an ingestible medicament into a gastrointestinal tract of a user, the method including:
providing to the user the device as described hereinabove, for ingestion by the user;
following the user ingesting the device, controlling the vibrating agitator such that the vibration mode of operation at least partially transpires within a region of the gastrointestinal tract in which the flowable ingestible medicament is absorbable by the body of the user, thereby to cause the periodic transitioning of the valve between the closed operative orientation and the open operative orientation, and delivery of the flowable ingestible medicament from the reservoir, via the conduit and the portal, into the environment surrounding the device.

In some embodiments, controlling the vibrating agitator further includes controlling the vibrating agitator such that the vibration mode of operation at least partially transpires within at least one of an estimated absorption time period and an actual absorption time period of the ingestible medicament within the gastrointestinal tract of the user.

In accordance with yet another embodiment of the present invention, there is provided a medicament delivery compartment, adapted to be attached to a vibrating ingestible capsule having a first housing portion and adapted to operate in a vibrating mode of operation, for delivery of a flowable ingestible medicament into the gastrointestinal tract of a user, the medicament delivery compartment including:
a second housing portion adapted to be attached to the first housing portion of the vibrating ingestible capsule, and having a portal formed therein;
a flexible and collapsible medicament reservoir dimensioned to contain the flowable ingestible medicament;
a reservoir biasing mechanism adapted to apply pressure to the flexible and collapsible medicament reservoir;
a conduit extending from the medicament reservoir to the portal, and sealing the portal; and
a valve including a weight and a valve biasing mechanism adapted, in a closed operative orientation, to bias the weight against the conduit so as to block flow through the conduit, and in an open operative orientation to remove the weight from the conduit so as to allow fluid to flow through the conduit.

In some embodiments, the valve is configured to be in the open operative orientation and to enable flow through the conduit when the vibrating ingestible capsule is in the vibration mode of operation. In some embodiments, at least one valve parameter of the valve is set such that the valve functions as a gear reducer, opening and closing at a frequency smaller than a frequency of vibration of the vibrating ingestible capsule. In some embodiments, the at least one valve parameter includes a mass of the weight, a length of the valve biasing mechanism, and a spring constant of the valve biasing mechanism.

In some embodiments, the second housing portion includes an attachment mechanism for mutual attachment to a corresponding attachment mechanism of the first housing portion of the vibrating ingestible capsule. In some embodiments, the second housing portion is adapted to be fixedly attached to the first housing portion. In some embodiments, the second housing portion is adapted to be attached to the first housing portion by snap fit engagement. In some embodiments, the second housing portion is adapted to be attached to the first housing portion by threaded engagement. In some embodiments, the second housing portion is adapted to be attached to the first housing portion by adhering. In some embodiments, the second housing portion is adapted to be attached to the first housing portion by soldering.

In some embodiments, a hollow formed in the second housing portion has a volume in the range of 200 mm³ to 3000 mm³, 200 mm³ to 800 mm³, 100 mm³ to 600 mm³, 400 mm³ to 1000 mm³, 700 mm³ to 1500 mm³, or 1400 mm³ to 3000 mm³.

In some embodiments, the flexible and collapsible medicament reservoir has a maximal volume in the range of 1 mm³ to 600 mm³, 1 mm³ to 10 mm³, 5 mm³ to 20 mm³, 15 mm³ to 50 mm³, 30 mm³ to 200 mm³, 100 mm³ to 400 mm³, or 300 mm³ to 600 mm³

In some embodiments, the flexible and collapsible medicament reservoir has a Young's modulus smaller than 1 GPa. In some embodiments, the flexible and collapsible medicament reservoir is formed of a material selected from the group consisting of: silicone rubber, natural rubber, Polyethylene, and PVC.

In some embodiments, the reservoir biasing mechanism includes a reservoir spring terminating in a pressure applying surface, the pressure applying surface engaging an exterior surface of the medicament reservoir. In some embodiments, the reservoir spring is anchored to the second housing portion. In some embodiments, the reservoir biasing mechanism has a spring constant in the range of 1 N/m to 200 N/m.

In some embodiments, the conduit is integrally formed with the medicament reservoir. In some embodiments, the conduit is formed of a different material than the medicament reservoir.

In some embodiments, a recovery time of the conduit is at most 0.1 seconds. In some embodiments, the vibrating ingestible capsule is adapted to vibrate at a frequency f, and a recovery time of the conduit is at most equal to 1/f. In some embodiments, the valve biasing mechanism has a frequency fv of moving between the closed operative orientation and the open operative orientation, and a recovery time of the conduit is at most equal to 1/fv.

In some embodiments, the conduit has a diameter in the range of 0.01 mm to 0.9 mm. In some embodiments, the conduit has a length in the range of 3 mm to 25 mm. In some embodiments, the conduit is at least one of, and optionally both of, flexible and resilient.

In some embodiments, the valve biasing mechanism is adapted to be anchored to the vibrating ingestible capsule. In some embodiments, the valve biasing mechanism is anchored to the second housing portion. In some embodiments, the valve biasing mechanism has a spring constant in the range of 0.1 N/m to 5 N/m.

In some embodiments, the weight has a mass in the range of 0.1 grams to 2 grams.

In some embodiments, the medicament delivery compartment further includes the flowable ingestible medicament disposed within the medicament reservoir.

In some embodiments, the flowable ingestible medicament has a viscosity in the range of 100 Pa·s to 1000 Pa·s.

In some embodiments, the flowable ingestible medicament is absorbable in the stomach of the user. In some embodiments, the flowable ingestible medicament is absorbable in the small intestine of the user.

In some embodiments, the flowable ingestible medicament is suitable for treatment of one or more symptom or disease, selected from the group consisting of: Parkinsonism; Parkinson's Disease; progressive supranuclear palsy; corticobasal degeneration; multiple system atrophy; Parkinson-plus syndromes; any neurodegenerative disease in which the subject exhibits at least one, at least two, or at least three of the classical features of Parkinson's disease: tremor, postural instability, and akinesia or bradykesia; any neurodegenerative disease in which the subject positively responds to a dopaminergic treatment; any neurodegenerative disease in which the particular subject positively responds to an anticholinergic treatment; Constipation; Crohn's disease; Gastroparesis; irritable bowel syndrome (IBS); diarrhea or loose bowel movements; colitis; Hirschsprung's disease; Dyspepsia; and dysphagia.

In some embodiments, the flowable ingestible medicament includes or includes an ingestible medicament selected from the group consisting of: Levodopa; at least one dopaminergic agent; at least one catecholamine precursor; a dopamine precursor; at least one dopamine precursor agent; (L)-3,4-dihydroxyphenylalanine; N-methyl-N-(2-propynyl)-2-methyl-1-phenylethyl-2-amine; tyrosine hydroxylase; apomorphine; at least one anticholinergic agent; at least one agent selected to antagonize at least one cholinergic receptor; benzhexol; orphenadrine; at least one selective allosteric potentiator of metabotropic glutamate receptor 4 (mGluR4); N-phenyl-7-(hydroxylimino)cyclopropa[b]chromen-1a-carboxamide; at least one osmotic agent; magnesium citrate; magnesium hydroxide; polyethylene glycol; sodium phosphate; MiraLAX®; at least one contraction stimulating agent; bisacodyl; senna; Correctol; Ducodyl; Dulcolax; Senexon; Senokot; at least one stool softening agent; docusate sodium; Colace; Linaclotide; Lactulose; Lubiprostone; Plecanatide; Prucaltride; Loperamide; and bismuth subsalicylate.

In accordance with a further embodiment of the present invention, there is provided a vibrating ingestible capsule adapted to be attached to a medicament delivery compartment for delivering a flowable ingestible medicament disposed within the medicament delivery compartment into the gastrointestinal tract of a user, the vibrating ingestible capsule including:
a housing including an attachment mechanism adapted for mutual attachment to a corresponding attachment mechanism of the medicament delivery compartment;
a vibrating agitator disposed within the housing and adapted such that, in a vibration mode of operation, the housing exerts vibrations on an environment surrounding the vibrating ingestible capsule;
a power supply disposed within the housing and adapted to power the vibrating agitator; and
a control element adapted to activate the vibrating agitator to be operative in the vibration mode of operation,
wherein at least one vibration parameter of the vibrating agitator is set so as to promote at least one of delivery of the flowable ingestible medicament from the medicament delivery compartment into an environment surrounding the medicament delivery compartment and absorption of the flowable ingestible medicament into the bloodstream of the user.

In some embodiments, the at least one vibration parameter includes at least one of a vibration frequency, a cumulative vibration duration, a number of vibration cycles per time unit, a duration of a vibration duration within a vibration cycle, a duration of a repose duration within a vibration cycle, a total duration of a single vibration cycle, and a net force exerted by the housing on the environment.

In some embodiments, the control element is adapted to control a timing or activation delay of the vibration mode of operation such that the vibration mode of operation at least partially transpires within a region of the gastrointestinal tract in which the flowable ingestible medicament is absorbable by the body of the user.

In some embodiments, the control element is adapted to control a timing or activation delay of the vibration mode of operation such that the vibration mode of operation at least partially transpires within at least one of an estimated absorption time period and an actual absorption time period of the flowable ingestible medicament within the gastrointestinal tract of the user.

In some embodiments, the control element is adapted to activate the vibrating agitator to be operative in the vibration mode of operation in response to receipt of an activation input.

In some embodiments, the vibrating ingestible capsule further includes at least one sensor adapted to provide the activation input to the control element.

In some embodiments, the at least one sensor includes an illumination sensor, adapted to provide an input indicating transition of the vibrating ingestible capsule from an illuminated environment to a dark environment.

In some embodiments, the at least one sensor includes a pressure sensor, adapted to provide an input indicating pressure applied to the vibrating ingestible capsule, which pressure is indicative of the vibrating ingestible capsule moving through a pharynx of the user.

In some embodiments, the at least one sensor includes a temperature sensor, adapted to provide an input indicating transition of the vibrating ingestible capsule from an area with ambient temperature to an area with a human body temperature.

In some embodiments, the at least one sensor includes an accelerometer, adapted to provide an input in response to a detected activation motion carried out with the vibrating ingestible capsule.

In some embodiments, the at least one sensor includes a moisture sensor, adapted to provide an input indicating transition of the vibrating ingestible capsule from a dry environment to a humid environment.

In some embodiments, the vibrating ingestible capsule is functionally associated with a control unit remote from the vibrating ingestible capsule, and wherein the control element is adapted to receive the activation input from the control unit.

In some embodiments, the control element is adapted to receive the activation input following ingesting of the vibrating ingestible capsule.

In some embodiments, the control element is adapted to receive the activation input prior to ingesting of the vibrating ingestible capsule.

In some embodiments, the control element is adapted to receive the activation input by receiving a vibration protocol to be used by the control element to control operation of the vibrating agitator.

In some embodiments, the vibrating agitator includes at least a radial agitation mechanism adapted, in the vibration mode of operation, to exert radial forces on the housing, in a radial direction with respect to a or the longitudinal axis of the housing, thereby to cause the vibrations exerted by the housing.

In some embodiments, the vibrating agitator includes at least an axial agitation mechanism adapted, in the vibration mode of operation, to exert axial forces on the housing, in an axial direction with respect to a or the longitudinal axis of the housing, thereby to cause the vibrations exerted by the housing.

In some embodiments, the vibrating agitator includes a radial agitation mechanism adapted, in the vibration mode of operation, to exert radial forces on the housing, in a radial direction with respect to a or the longitudinal axis of the housing, and a separate axial agitation mechanism adapted, in the vibration mode of operation, to exert axial forces on the housing, in an axial direction with respect to a or the longitudinal axis of the housing.

In some embodiments, the vibrating agitator includes a single agitation mechanism adapted, in the vibration mode of operation, to exert radial forces on the housing in a radial direction with respect to a or the longitudinal axis of the housing, and to exert axial forces on the housing, in an axial direction with respect to the longitudinal axis of the housing.

In some embodiments, the control element is adapted to control the vibrating agitator such that the vibrating mode of operation includes a plurality of cycles, each of the cycles including a vibration duration followed by a repose duration, wherein the housing exerts the vibrations during the vibration duration. In some embodiments, the repose duration is greater than the vibration duration.

In some embodiments, the vibration duration is in the range of 0.1 second to 10 seconds, 1 second to 10 seconds, 1 second to 9 seconds, 2 seconds to 9 seconds, 3 seconds to 9 seconds, 3 seconds to 8 seconds, 3 seconds to 7 seconds, 3 seconds to 6 seconds, 4 seconds to 6 seconds, or 5 seconds to 6 seconds.

In some embodiments, the repose duration is in the range of 1 second to 180 seconds, 3 seconds to 180 seconds, 5 seconds to 180 seconds, 5 seconds to 150 seconds, seconds to 120 seconds, 8 seconds to 100 seconds, 8 seconds to 30 seconds, 10 seconds to 80 seconds, 10 seconds to 70 seconds, 10 seconds to 60 seconds, 10 seconds to 50 seconds, 10 seconds to 40 seconds, 10 seconds to 30 seconds, 10 seconds to 20 seconds, or 15 seconds to 20 seconds.

In some embodiments, a duration of each of the plurality of cycles is in the range of 1.1 seconds to 200 seconds, 5 seconds to 200 seconds, 10 seconds to 200 seconds, 10 seconds to 150 seconds, 10 seconds to 100 seconds, 10 seconds to 80 seconds, 10 seconds to 50 seconds, 10 seconds to 40 seconds, 10 seconds to 30 seconds, 15 seconds to 50 seconds, 15 seconds to 40 seconds, 15 seconds to 30 seconds, or 15 seconds to 25 seconds.

In some embodiments, the control element is adapted to control the vibrating agitator such that a cumulative duration of the vibrating mode of operation is in the range of 1 hour to 12 hours, 2 hours to 10 hours, 2 hours to 8 hours, 2 hours to 6 hours, 2 hours to 4 hours, or 2 hours to 3 hours.

In some embodiments, the vibrating agitator is configured to exert forces on the housing of the vibrating ingestible capsule, such that a net force exerted by the housing on the environment thereof is in the range of 50 grams force (gf) to 600 gf, 50 gf to 550 gf, 100 gf to 550 gf, 100 gf to 500 gf, 150 gf to 500 gf, 200 gf to 500 gf, or 200 gf to 450 gf.

In some embodiments, the vibrating agitator is configured to exert the forces on the housing to attain a housing vibrational frequency within a range of 10 Hz to 650 Hz, 15 Hz to 600 Hz, 20 Hz to 550 Hz, 30 Hz to 550 Hz, 50 Hz to 500 Hz, 70 Hz to 500 Hz, 100 Hz to 500 Hz, 130 Hz to 500 Hz, or 150 Hz to 500 Hz.

In some embodiments, the housing is adapted to be fixedly attached to a second housing of the medicament delivery compartment. In some embodiments, the housing is adapted to be attached to the medicament delivery compartment by snap fit engagement. In some embodiments, the housing is adapted to be attached to the medicament delivery compartment by threaded engagement. In some embodiments, the housing is adapted to be attached to the medicament delivery compartment by adhering. In some embodiments, the housing is adapted to be attached to the medicament delivery compartment by soldering.

In accordance with another further embodiment of the present invention, there is provided a method for delivering a flowable ingestible medicament into the gastrointestinal tract of a user, the method including:
providing an ingestible device including:
   a housing including a first housing portion and a second housing portion, the second housing portion having a portal formed therein;
   a vibrating agitator disposed within the housing and adapted such that, in a vibration mode of operation, the housing exerts vibrations on an environment surrounding the ingestible device;
   a power supply disposed within the housing and adapted to power the vibrating agitator; and
   a control element adapted to activate the vibrating agitator to be operative in the vibration mode of operation;
   a flexible and collapsible medicament reservoir having the flowable ingestible medicament disposed therein;
   a reservoir biasing mechanism applying pressure to the flexible and collapsible medicament reservoir;
   a conduit extending from the medicament reservoir to the portal, and sealing the portal; and
   a valve including a weight and a valve biasing mechanism adapted, in a closed operative orientation, to bias the weight against the conduit so as to block flow through the conduit, and in an open operative orientation to remove the weight from the conduit so as to allow fluid to flow through the conduit, the valve being functionally associated with at least one of the housing and the vibrating agitator,
ingesting the ingestible device by the user; and
following the user ingesting the device, controlling the vibrating agitator such that in the vibration mode of operation, at least some of the vibrations exerted by the vibrating agitator or by the housing are applied to the valve biasing mechanism and periodically transition the valve between the closed operative orientation and the open operative orientation,
wherein, during the controlling, when the vibrating agitator is in the vibration mode of operation and the valve is in the open operative orientation, pressure applied by the reservoir biasing mechanism to the reservoir causes the flowable ingestible medicament to flow from the reservoir, via the conduit and the portal, to an environment surrounding the assembly.

In some embodiments, providing includes providing the ingestible device wherein the first housing portion and the second housing portion include a single housing including the portal and defining a single hollow, the single hollow having disposed therein the vibrating agitator, the power supply, the control element, the flexible and collapsible medicament reservoir, the reservoir biasing mechanism, and the conduit. In some embodiments, the weight includes the vibrating agitator.

In accordance with yet another embodiment of the present invention, there is provided a method for delivering a flowable ingestible medicament into the gastrointestinal tract of a user, the method including:
providing a vibrating ingestible capsule including:
   a housing;
   a vibrating agitator disposed within the housing and adapted such that, in a vibration mode of operation, the housing exerts vibrations on an environment surrounding the vibrating ingestible capsule;
   a power supply disposed within the housing and adapted to power the vibrating agitator; and
   a control element adapted to activate the vibrating agitator to be operative in the vibration mode of operation; and
forming an ingestible device by attaching to the vibrating ingestible capsule a medicament delivery compartment, the medicament delivery compartment including:
   a second housing portion having a portal formed therein;
   a flexible and collapsible medicament reservoir having the flowable ingestible medicament disposed therein;
   a reservoir biasing mechanism applying pressure to the flexible and collapsible medicament reservoir;
   a conduit extending from the medicament reservoir to the portal, and sealing the portal; and
   a valve including a weight and valve biasing mechanism adapted, in a closed operative orientation, to bias the weight against the conduit so as to block flow through the conduit, and in an open operative orientation to remove the weight from the conduit so as to allow fluid to flow through the conduit, the valve being functionally associated with at least one of the housing and the vibrating agitator;
ingesting the ingestible device by the user; and
following the user ingesting the device, controlling the vibrating ingestible capsule such that in the vibration mode of operation, at least some of the vibrations exerted by the vibrating agitator or by the housing are applied to the valve biasing mechanism and periodically transition the valve between the closed operative orientation and the open operative orientation,
wherein, during the controlling, when the vibrating agitator is in the vibration mode of operation and the valve is in the open operative orientation, pressure applied by the reservoir biasing mechanism to the reservoir causes the flowable ingestible medicament to flow from the reservoir, via the conduit and the portal, to an environment surrounding the device.

In some embodiments, the method further includes, prior to the attaching, filling the medicament reservoir with the flowable ingestible medicament.

In some embodiments, controlling the vibrating ingestible capsule includes setting at least one vibration parameter of the vibrating ingestible capsule so as to promote transitioning of the valve between the closed operative orientation and the open operative orientation.

In some embodiments, controlling the vibrating ingestible capsule includes setting at least one vibration parameter of the vibrating ingestible capsule so as to promote absorption of the ingestible medicament into the bloodstream of the user.

In some embodiments, setting the at least one vibration parameter includes setting at least one of a vibration frequency, a cumulative vibration duration, a number of vibration cycles per time unit, a duration of a vibration duration within a vibration cycle, a duration of a repose duration within a vibration cycle, a total duration of a single vibration cycle, and a net force exerted by the housing on the environment.

In some embodiments, the housing of the vibrating ingestible capsule includes an attachment mechanism, the second housing portion includes a corresponding attachment mechanism, and the attaching includes mutually attaching the housing of the vibrating ingestible capsule to the second housing portion.

In some embodiments, the attaching includes fixedly attaching the second housing portion to the vibrating ingestible capsule. In some embodiments, the attaching includes attaching the second housing portion to the vibrating ingestible capsule by snap fit engagement. In some embodiments, the attaching includes attaching the second housing portion to the vibrating ingestible capsule by threaded engagement. In some embodiments, the attaching includes attaching the second housing portion to the vibrating ingestible capsule by adhering. In some embodiments, the attaching includes attaching the second housing portion to the vibrating ingestible capsule by soldering.

In some embodiments, the controlling includes controlling a timing or activation delay of the vibration mode of operation such that the vibration mode of operation at least partially transpires within a region of the gastrointestinal tract in which the flowable ingestible medicament is absorbable by the body of the user.

In some embodiments, the controlling includes controlling a timing or activation delay of the vibration mode of operation such that the vibration mode of operation at least partially transpires within at least one of an estimated absorption time period and an actual absorption time period of the flowable ingestible medicament released from reservoir within the gastrointestinal tract of the user.

In some embodiments, the method further includes, prior to the controlling, at the control element receiving an activation input, and the controlling includes activating the vibrating agitator to be operative in the vibration mode of operation following the receiving the activation input.

In some embodiments, the receiving the activation input includes receiving the activation input from at least one sensor.

In some embodiments, the receiving the activation input includes receiving, from an illumination sensor, an input indicating transition of the assembly from an illuminated environment to a dark environment.

In some embodiments, the receiving the activation input includes receiving, from a pressure sensor, an input indicating pressure applied to the assembly, which pressure is indicative of the assembly moving through a pharynx of the user.

In some embodiments, the receiving the activation input includes receiving, from a temperature sensor, an input indicating transition of the assembly from an area with ambient temperature to an area with a human body temperature.

In some embodiments, the receiving the activation input includes receiving, from an accelerometer, an input in response to a detected activation motion carried out with the assembly.

In some embodiments, the receiving the activation input includes receiving, from a moisture sensor, an input indicating transition of the assembly from a dry environment to a humid environment.

In some embodiments, the receiving the activation input includes receiving the activation input from a control unit remote from the assembly and functionally associated with the control element.

In some embodiments, the receiving the activation input occurs following the ingesting of the assembly.

In some embodiments, the receiving the activation input occurs prior to the ingesting of the assembly.

In some embodiments, the receiving the activation input includes receiving a vibration protocol to be used by the control element for the controlling operation of the vibrating agitator.

In some embodiments, the controlling the vibrating agitator includes controlling the vibrating agitator such that the vibrating mode of operation includes a plurality of cycles, each of the cycles including a vibration duration followed by a repose duration, wherein the housing exerts the vibrations during the vibration duration. In some embodiments, the repose duration is greater than the vibration duration.

In some embodiments, the vibration duration is in the range of 0.1 second to 10 seconds, 1 second to 10 seconds, 1 second to 9 seconds, 2 seconds to 9 seconds, 3 seconds to 9 seconds, 3 seconds to 8 seconds, 3 seconds to 7 seconds, 3 seconds to 6 seconds, 4 seconds to 6 seconds, or 5 seconds to 6 seconds.

In some embodiments, the repose duration is in the range of 1 second to 180 seconds, 3 seconds to 180 seconds, 5 seconds to 180 seconds, 5 seconds to 150 seconds, seconds to 120 seconds, 8 seconds to 100 seconds, 8 seconds to 30 seconds, 10 seconds to 80 seconds, 10 seconds to 70 seconds, 10 seconds to 60 seconds, 10 seconds to 50 seconds, 10 seconds to 40 seconds, 10 seconds to 30 seconds, 10 seconds to 20 seconds, or 15 seconds to 20 seconds.

In some embodiments, a duration of each of the plurality of cycles is in the range of 1.1 seconds to 200 seconds, 5 seconds to 200 seconds, 10 seconds to 200 seconds, 10 seconds to 150 seconds, 10 seconds to 100 seconds, 10 seconds to 80 seconds, 10 seconds to 50 seconds, 10 seconds to 40 seconds, 10 seconds to 30 seconds, 15 seconds to 50 seconds, 15 seconds to 40 seconds, 15 seconds to 30 seconds, or 15 seconds to 25 seconds.

In some embodiments, the controlling includes controlling the vibrating agitator such that a cumulative duration of the vibrating mode of operation is in the range of 1 hour to 12 hours, 2 hours to 10 hours, 2 hours to 8 hours, 2 hours to 6 hours, 2 hours to 4 hours, or 2 hours to 3 hours.

In some embodiments, the controlling including controlling the vibrating agitator to exert forces on the housing of the vibrating ingestible capsule, such that a net force exerted by the housing on the environment thereof is in the range of 50 grams force (gf) to 600 gf, 50 gf to 550 gf, 100 gf to 550 gf, 100 gf to 500 gf, 150 gf to 500 gf, 200 gf to 500 gf, or 200 gf to 450 gf.

In some embodiments, the controlling including controlling the vibrating agitator to exert the forces on the housing to attain a housing vibrational frequency within a range of 10 Hz to 650 Hz, 15 Hz to 600 Hz, 20 Hz to 550 Hz, 30 Hz to 550 Hz, 50 Hz to 500 Hz, 70 Hz to 500 Hz, 100 Hz to 500 Hz, 130 Hz to 500 Hz, or 150 Hz to 500 Hz.

In some embodiments, the flowable ingestible medicament has a viscosity in the range of 100 Pa·s to 1000 Pa·s.

In some embodiments, the flowable ingestible medicament is absorbable in the stomach of the user. In some embodiments, the flowable ingestible medicament is absorbable in the small intestine of the user.

In some embodiments, the flowable ingestible medicament is suitable for treatment of one or more symptom or disease, selected from the group consisting of:
Parkinsonism; Parkinson's Disease; progressive supranuclear palsy; corticobasal degeneration; multiple system atrophy; Parkinson-plus syndromes; any neurodegenerative disease in which the subject exhibits at least one, at least two, or at least three of the classical features of Parkinson's disease: tremor, postural instability, and akinesia or bradykesia; any neurodegenerative disease in which the subject positively responds to a dopaminergic treatment; any neurodegenerative disease in which the particular subject positively responds to an anticholinergic treatment; Constipation; Crohn's disease; Gastroparesis; irritable bowel syndrome (IBS); diarrhea or loose bowel movements; colitis; Hirschsprung's disease; Dyspepsia; and dysphagia.

In some embodiments, the flowable ingestible medicament includes or includes an ingestible medicament selected from the group consisting of:
Levodopa; at least one dopaminergic agent; at least one catecholamine precursor; a dopamine precursor; at least one dopamine precursor agent; (L)-3,4-dihydroxyphenylalanine; N-methyl-N-(2-propynyl)-2-methyl-1-phenylethyl-2-amine; tyrosine hydroxylase; apomorphine; at least one anticholinergic agent; at least one agent selected to antagonize at least one cholinergic receptor; benzhexol; orphenadrine; at least one selective allosteric potentiator of metabotropic glutamate receptor 4 (mGluR4); N-phenyl-7-(hydroxylimino)cyclopropa[b]chromen-1a-carboxamide; at least one osmotic agent; magnesium citrate; magnesium hydroxide; polyethylene glycol; sodium phosphate; MiraLAX®; at least one contraction stimulating agent; bisacodyl; senna; Correctol; Ducodyl; Dulcolax; Senexon; Senokot; at least one stool softening agent; docusate sodium; Colace; Linaclotide; Lactulose; Lubiprostone; Plecanatide; Prucaltride; Loperamide; and bismuth subsalicylate.

### BRIEF DESCRIPTION OF THE FIGURES

The foregoing discussion will be understood more readily from the following detailed description of the invention, when taken in conjunction with the accompanying Figures, in which:

FIG. 1 is a schematic block diagram of a gastrointestinal treatment system including a vibrating ingestible capsule according to an embodiment of the present invention;

FIG. 2 is a schematic flowchart of a method for using a vibrating gastrointestinal capsule in the treatment of Parkinsonism, according to the present invention, the treatment being based on use of an ingestible vibrating gastrointestinal capsule, for example as shown in FIG. 1;

FIG. 3 is a schematic flowchart of a method for using a vibrating gastrointestinal capsule to improve or accelerate the absorption to the bloodstream of an ingestible medicament, or to improve the efficacy of such a medicament, according to the present invention, the method being based on use of an ingestible vibrating gastrointestinal capsule, for example as shown in FIG. 1;

FIG. 4 is a schematic block diagram of a device for delivering an ingestible medicament into the gastrointestinal tract of a user according to an embodiment of the present invention;

FIGS. 5A and 5B are, respectively, a perspective view illustration and a planar side view illustration of a first embodiment of a device for delivering an ingestible medicament into the gastrointestinal tract of a user, the device having a medicament tablet including the ingestible medicament disposed therein, according to an embodiment of the present invention;

FIGS. 6A and 6B are, respectively, a complete and a partial perspective sectional illustration of the device of FIGS. 5A and 5B, having the medicament tablet disposed therein;

FIGS. 7A and 7B are, respectively, a complete and a partial planar sectional illustration of the device of FIGS. 5A and 5B, having the medicament tablet disposed therein;

FIGS. 8A and 8B are, respectively, partial sectional illustrations of the device of FIGS. 5A and 5B, at two times during use thereof;

FIGS. 9A and 9B are, respectively, partial sectional illustrations of a device similar to the device of FIGS. 5A and 5B and having a radial medicament tablet biasing mechanism, at two times during use thereof, according to another embodiment of the present invention;

FIGS. 10A and 10B are, respectively, a perspective view illustration and a planar view illustration of a second embodiment of a device for delivering an ingestible medicament into the gastrointestinal tract of a user, the device having a medicament tablet including the ingestible medicament disposed therein, according to yet another embodiment of the present invention;

FIGS. 11A and 11B are, respectively, a complete and a partial planar sectional illustration of the device of FIGS. 10A and 10B, having the medicament tablet disposed therein;

FIG. 12 is a schematic flowchart of a method for delivering an ingestible medicament of a medicament tablet into the gastrointestinal tract of user according to the present invention, the method being based on use of any one of the devices of FIGS. 4 to 11B;

FIG. 13 is a schematic block diagram of a device for delivering a flowable ingestible medicament into the gastrointestinal tract of a user according to an embodiment of the present invention;

FIG. 14 is a planar sectional illustration of a device for delivering a flowable ingestible medicament into the gastrointestinal tract of a user according to another embodiment of the present invention, the device including a medicament reservoir and a valve;

FIG. 15 is a partial perspective sectional illustration of the device of FIG. 14;

FIGS. 16A and 16B are partial planar sectional illustrations of the device of FIG. 14, where the medicament reservoir is full, and the valve is in closed and open operative orientations, respectively;

FIG. 17 is a partial planar sectional illustrations of the device of FIG. 14, where the medicament reservoir is empty, and the valve is in a closed operative orientation;

FIG. 18 is a schematic diagram of a device for delivering a flowable ingestible medicament into the gastrointestinal tract of a user according to yet another embodiment of the present invention; and

FIG. 19 is a schematic flowchart of a method for delivering a flowable ingestible medicament into the gastrointestinal tract of user according to the present invention, the method being based on use of any one of the devices of FIGS. 13 to 18.

### DESCRIPTION OF THE PREFERRED EMBODIMENTS

The principles of the inventive gastrointestinal treatment system and method of using the inventive gastrointestinal treatment system in treatment of Parkinsonism, may be better understood with reference to the drawings and the accompanying description.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

For the purposes of this application, the terms “subject” and “user” are used interchangeably, and relate to a human.

For the purposes of this application, the term “vibrating ingestible capsule” relates to an ingestible capsule adapted to at least intermittently vibrate, for a cumulative duration of at least one minute, in accordance with a vibration protocol of the capsule.

For the purposes of this application, the terms “vibrating agitation mechanism” and “vibrating agitator” are used interchangeably, and refer to any type of mechanism that vibrates or causes elements in its vicinity to vibrate, including a vibration motor or engine and a pendulum, and a motor driven agitator such as a motor driven eccentric weight, a motor driven pendulum, and a motor driven axial agitator.

For the purposes of this application, the terms “intermittently activated vibrating agitation mechanism” and “intermittently activated vibrating agitator” are used interchangeably, and refer to a vibrating agitator that vibrates or causes elements in its vicinity to vibrate and is operative at certain times, and does not vibrate or cause elements in its vicinity to vibrate at other times, the activation times being selected by a control element, or other control unit controlling the vibrating agitator.

For the purposes of this application, the terms “control element”, “control mechanism” and “controller” are used interchangeably refer to a component for controlling operation of mechanical and/or electrical components of the capsule, which includes a processing unit functionally associated with a non-tangible computer readable storage medium. The storage medium stores instructions, which, when executed by the processing unit, cause the processing unit to carry out actions which control the operation of the mechanical and/or electrical components of the capsule. For example, the instructions may include instructions to activate operation of a vibrating agitator at a specific time, frequency, cycle, and/or for a specific duration. The control element may be functionally associated with, or may include, a transceiver for receiving input, which input may be used to trigger execution of specific instructions stored in the storage medium.

For the purposes of this application, the term “biasing mechanism” refers to any structure, or device, adapted to apply pressure to a second element, even when the position of the second element changes relative to an anchoring point of the structure or device. Biasing mechanisms include springs, such as compression springs and extension springs, as well as spring loaded leaves, plungers, and the like.

For the purposes of this application, the term “vibration protocol” relates to a protocol specifying vibration parameters of an intermittently activated vibrating agitator of a vibrating ingestible capsule. Typically, the vibration protocol relates to an activation delay for initiating vibration (e.g., a duration between “initial” activation of the capsule and the first activation of the vibration engine), a vibration rate (number of vibration cycles per hour), a vibration duration and a repose duration for each vibration cycle, a vibration frequency, an amount of force exerted by the vibrations, and the like.

For the purposes of this application, the term “treatment procedure” relates to parameters of a treatment utilizing vibrating ingestible capsules, which are typically defined by a treating physician or medical practitioner. For example, the treatment procedure may include the number of capsules to be taken within a specific time duration (e.g., 3 capsules per week, 2 capsules per day), the frequency at which capsules should be taken, the time of day at which capsules should be taken, whether the capsule should be taken with or without food, and the like.

For the purpose of this application, the term “treatment protocol” relates to all aspects of treatment of a subject with a vibrating ingestible capsule, and includes the treatment procedure as well as the vibration protocol to be used for treating the subject.

For the purpose of this application, the term “activation input” relates to an input received by a control element of a vibrating ingestible capsule, which causes the control element of the capsule to activate itself, so as to be able to process inputs and/or to control additional components of the capsule. The activation input may be received from an element forming part of the capsule, such as a sensor sensing specific conditions in which the capsule should be activated, or from a remote source, such as a remote control element, for example by way of a signal transmitted to the capsule, magnetic field applied to the capsule, specific motion applied to the capsule, or any other type of input provided to the capsule from a remote source. The activation input may be provided prior to the subject ingesting the capsule or may be provided while the capsule is traversing the GI tract of the subject.

For the purpose of this application, a vibrating ingestible capsule is said to be in an “inoperative state” when the capsule is in a storage condition, intended to preserve the life of a battery thereof. In the inoperative state, components of the capsule which are intended to receive or to provide an activation input, such as specific sensors, transceivers, and/or timing mechanisms may be active at least to a minimal degree. However, in the inoperative state, no vibration takes place, and a control element controlling vibration of the capsule is inactive.

For the purpose of this application, a vibrating ingestible capsule is said to be in an “operative state” when the control element of the capsule is processing inputs and data, and can cause a vibrating agitator of the capsule to vibrate or cause elements in its vicinity to vibrate.

For the purposes of this application, an action carried out on or in an object is considered to be “maintaining the integrity” of the object, if, during the action and for a duration of at least 24 hours following the action there is no permanent change to the structure of the object, or of any portion of the object, other than movement of movable portions within the object. Specifically, vibration of a vibrating ingestible capsule is considered to maintain the integrity of the vibrating ingestible capsule if, during and following such vibration, there is no permanent change to the mechanical structure of the capsule, such as to the housing thereof, with the exception of movement of the vibrating agitation mechanism or a portion thereof within the capsule.

A test for determining whether or not vibration of a vibrating ingestible capsule is considered to maintain the integrity of the vibrating ingestible capsule or of a housing thereof includes placing the vibrating ingestible capsule in a 250 ml cup of water, where the water is maintained at a human body temperature—i.e. in the range of 36° C. to 37.5° C. The vibration of the capsule is activated, and is allowed to continue for a duration of 30 hours, or until the power source is depleted, in which case the capsule remains in the water following depletion of the power source till the duration of 30 hours is completed. If the mechanical structure of the housing or of capsule is in no way damaged or changed at the end of the 30 hours, the vibration of the capsule maintains the integrity of the housing or of the capsule, respectively.

For the purpose of this application, an “ingestible medicament” is at least partially absorbable to the bloodstream from within the stomach, small intestine, and large intestine, and more typically, within the stomach or small intestine.

For the purpose of this application, the term “partially absorbable” is meant to include the possibility that the environment within the gastrointestinal tract (including acids, enzymes, etc. thereof) may chemically modify the ingested medicament in order to achieve the characteristic “partially absorbable”.

For the purposes of this application the term “flowable ingestible medicament” relates to any dosage form of an ingestible medicament which can flow through a conduit, such as a liquid ingestible medicament, a suspension of an ingestible medicament, a gaseous ingestible medicament, a solution of an ingestible medicament, a dissolved ingestible medicament, a melted ingestible medicament, and the like.

For the purposes of this application the term “medicament tablet” relates to any dosage form of an ingestible medicament, in which the ingestible medicament maintains a pre-defined closed contour. This may include a pill, a tablet, a capsule, a liquid-gel capsule, or compressed powder.

For the purposes of this application, the disclosure of a commercial name of a material or drug is meant to be a disclosure of the corresponding generic material or drug, and of the active ingredient(s) within the commercial material or drug and/or within the corresponding generic material or drug.

For the purpose of this application, an estimated absorption time may be determined as follows:
(i) ingestible medicaments that are absorbed in the stomach have an estimated absorption time within a range of 0.5 to 1.5 hours from the time of ingestion of the ingestible medicament;
(ii) ingestible medicaments that are absorbed in the small intestine have an estimated absorption time within a range of 1.0 to 5 hours from the time of ingestion of the ingestible medicament;
(iii) ingestible medicaments that are absorbed in both the stomach and the small intestine have an estimated absorption time within a range of 0.5 to 5 hours from the time of ingestion of the ingestible medicament;
(iv) ingestible medicaments that are absorbed in the large intestine have an estimated absorption time of at least 4 hours, and more typically, within a range of 4 to hours, 6 to 30 hours, 6 to 20 hours, or 8 to 20 hours from the time of ingestion of the ingestible medicament.

The location within the GI tract at which the particular ingestible medicament is absorbed to the bloodstream may often be public knowledge. This location may be provided by, or known to, the manufacturer and/or distributor of the particular ingestible medicament. Alternatively or additionally, the location may be known to relevant medical practitioners, including doctors and pharmacists, and more particularly, to a medical practitioner of the user.

For the purpose of this application, an actual absorption time may be determined from clinical data, in vivo or in vitro, according to accepted clinical procedures known to those of skill in the art. Since actual absorption is achieved over a period of time, the “actual absorption time” or “actual absorption time period” may be defined by the time period at which between 20% and 80% of the absorption occurs. In the absence of such data, the “actual absorption time” or “actual absorption time period” may be defined by determining the “peak” actual absorption time, and building a time period of up to 1 hour on each side of the peak time.

For the purpose of this application, the term “Parkinsonism” is meant to include Parkinson's disease, or symptoms of neurodegeneration associated therewith.

For the purpose of this application, the term “Parkinsonism” is meant to include progressive supranuclear palsy, or symptoms of neurodegeneration associated therewith.

For the purpose of this application, the term “Parkinsonism” is meant to include corticobasal degeneration, or symptoms of neurodegeneration associated therewith.

For the purpose of this application, the term “Parkinsonism” is meant to include multiple system atrophy, or symptoms of neurodegeneration associated therewith.

For the purpose of this application, the term “Parkinsonism” is meant to include Parkinson-plus syndromes (also known as disorders of multiple system degeneration), or symptoms of neurodegeneration associated therewith.

For the purpose of this application, the term “Parkinsonism” is meant to include any neurodegenerative disease in which the subject exhibits at least one (and typically at least two or three) of the classical features of Parkinson's disease: tremor, postural instability, and akinesia or bradykesia.

For the purpose of this application, the term “Parkinsonism” is meant to include any neurodegenerative disease in which the subject positively responds to a dopaminergic treatment.

For the purpose of this application, the term “Parkinsonism” is meant to include any neurodegenerative disease in which the particular subject positively responds to an anticholinergic treatment.

For the purpose of this application, the term “Parkinsonism” is meant to include any neurodegenerative disease in which a dopaminergic treatment is clinically utilized to treat the sufferers or subjects.

For the purpose of this application, the term “Parkinsonism” is meant to include any neurodegenerative disease in which an anticholinergic treatment is clinically utilized to treat the sufferers or subjects.

For the purpose of this application, the term “Parkinson's disease” (PD) is meant as used by those of skill in the art of neurodegenerative diseases. It is believed that PD is due to the loss of brain cells that produce dopamine. Early signs and symptoms of Parkinson's disease include at least one of tremors (or trembling), slowness of movement, body rigidity and stiffness, and gait problems.

For the purpose of this application, the term “treatment of Parkinsonism” and the like refers to at least one of: (i) delaying onset of Parkinsonism (e.g., PD); (ii) mitigating the development of Parkinsonism (e.g., PD); and (iii) managing a condition of Parkinsonism (e.g., PD).

For the purpose of this application, the term “ailment of the GI tract” is meant to include chronic or acute constipation, or symptoms associated therewith.

For the purpose of this application, the term “ailment of the GI tract” is meant to include gastroparesis, or symptoms associated therewith.

For the purpose of this application, the term “ailment of the GI tract” is meant to include Crohn's disease, or symptoms associated therewith.

For the purpose of this application, the term “ailment of the GI tract” is meant to include chronic or acute diarrhea, or symptoms associated therewith.

For the purpose of this application, the term “ailment of the GI tract” is meant to include colitis, or symptoms associated therewith.

For the purpose of this application, the term “ailment of the GI tract” is meant to include dyspepsia or dysphagia, or symptoms associated therewith.

For the purpose of this application, the term “ailment of the GI tract” is meant to include Hirschsprung's disease, or symptoms associated therewith.

For the purpose of this application, the term “ailment of the GI tract” is meant to include irritable bowel syndrome, or symptoms associated therewith.

For the purpose of this application, the term “ailment of the GI tract” is meant to include any disease in which the subject positively responds to an osmotic gastrointestinal treatment.

For the purpose of this application, the term “ailment of the GI tract” is meant to include any disease in which the particular subject positively responds to a stool softening treatment.

For the purpose of this application, the term “ailment of the GI tract” is meant to include any disease in which the particular subject positively responds to a GI contraction inducing treatment.

For the purpose of this application, the term “ailment of the GI tract” is meant to include any disease in which the subject positively responds to a GI fluid absorption inducing treatment.

For the purpose of this application, the term “managing a condition of”, with respect to Parkinsonism and the like, is meant to include, inter alia, improving absorption of a medicament such as a medicament used in the treatment of Parkinsonism (e.g., levodopa) into the bloodstream. Such condition management may be manifested by at least one of (i) improved medicament efficacy due to the increased absorption; and (ii) reduced dosage of the medicament, due to the increased medicament absorption efficacy.

For the purpose of this application, the term “managing a condition of”, with respect to an ailment of the GI tract, is meant to include, inter alia, improving absorption of a medicament, such as a medicament used in the treatment of the ailment of the GI tract (e.g., Linaclotide (Linzess®)), into the bloodstream. Such condition management may be manifested by at least one of (i) improved medicament efficacy due to the increased absorption; and (ii) reduced dosage of the medicament, due to the increased medicament absorption efficacy.

For the purpose of this application, a first element is said to envelop a second element, if the second element is disposed within the first element, and the first element fully surrounds the second element. The second element need not be attached to the first element, and may be movable relative to the first element, within the first element, but may also be attached to the first element.

For the purpose of this application, a first element is said to partially envelop a second element, if at least part of the exterior surface of the second element is covered by the first element or is surrounded by the first element. The second element need not be attached to the first element, and may be movable relative to the first element, within the first element, but may also be attached to the first element.

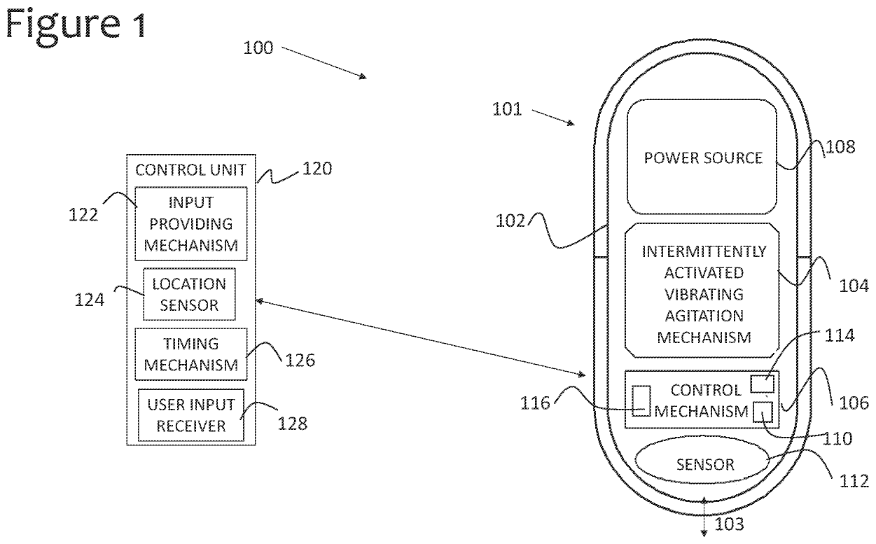

Referring now to the drawings, FIG. 1 is a schematic block diagram of a gastrointestinal treatment system 100 including a vibrating ingestible capsule 101 according to an embodiment of the present invention.

As seen in FIG. 1, gastrointestinal treatment system 100 includes vibrating ingestible capsule 101. Capsule 101 includes a capsule housing or shell 102, arranged along a longitudinal axis 103 and having disposed therein a vibrating agitation mechanism 104. A control mechanism 106, which may for example be, or include, a processor, is adapted to control operation of vibrating agitation mechanism 104, and at least one power source 108 provides power to vibrating agitation mechanism 104 and control mechanism 106.

Power source 108 may be any suitable power source, such as one or more alkaline or silver oxide batteries, primary batteries, rechargeable batteries, capacitors and/or supercapacitors.

Intermittently activated vibrating agitation mechanism 104 is adapted to have a vibration mode of operation (also termed the first mode of operation) and a rest mode of operation (also termed the second mode of operation). In the vibration mode of operation, intermittently activated vibrating agitation mechanism 104 is adapted to exert forces on capsule housing 102, such that capsule housing 102 exerts vibrations on an environment surrounding capsule 101, while maintaining the integrity of capsule 101 and of the capsule housing 102.

In some embodiments, the capsule is in an inoperative state, until the receipt of an activation input, which causes control mechanism 106 to transition the capsule from the inoperative state to an operative state.

In some embodiments, control mechanism 106 is functionally associated with, or includes, a timing mechanism 110, powered by power source 108 and adapted to track at least one time characteristic, such as a duration that has passed since an activation input was received, or a duration that has passed since the subject ingested capsule 101.

In some embodiments, capsule 101 is devoid of any sensors for sensing an environment thereof. In some such embodiments, control mechanism 106 is adapted, in response to receipt of an activation input, to wait a predetermined delay time, and following the predetermined delay time, to activate vibrating agitation mechanism 104 to operate in said first vibration mode of operation.

In some embodiments, the predetermined delay time may be in the range of 5 minutes to 15 minutes, 5 minutes to 30 minutes, 5 minutes to 45 minutes, 5 minutes to 1 hour, 2 hours to 12 hours, 4 hours to 12 hours, 6 hours to 12 hours, 8 hours to 12 hours, 12 hours to 24 hours, 24 hours to 36 hours, 36 hours to 48 hours, or 48 hours to 72 hours.

In other embodiments, such as the embodiment illustrated in FIG. 1, capsule 101 further includes at least one sensor 112, functionally associated with control mechanism 106. The at least one sensor 112 may be adapted to sense at least one parameter within capsule 101 or in an environment of capsule 101, and may include a temperature sensor, an illumination sensor, a moisture sensor, a pressure sensor, an accelerometer, or any other suitable sensor. In some embodiments, the at least one sensor 112 is adapted to identify a specific condition in capsule 101 or in the vicinity thereof, and to provide an activation input to control mechanism 106 in response to identification of the condition. For example, in some embodiments the condition is indicative of the subject ingesting capsule 101.

For example, in some embodiments sensor 112 may include an illumination sensor, adapted to identify transition of capsule 101 from an illuminated environment (e.g. outside the human body) to a dark environment (e.g. within the human body) and to provide an activation input in response to identification of such a transition.

As another example, in some embodiments sensor 112 may include a motion or acceleration sensor, such as an accelerometer, adapted to identify an activation motion carried out by a user on capsule 101 and to provide an activation input in response to identification of such a transition. An example of an accelerometer providing an activation input for a gastrointestinal capsule is provided in U.S. patent application Ser. No. 15/168,065, filed on May 29, 2016, which is incorporated by reference for all purposes as if fully set forth herein.

As another example, in some embodiments sensor 112 may include a pressure sensor adapted identify pressure applied to the capsule 101, which pressure is indicative of the capsule moving through a pharynx of the subject, and to provide an activation input in response to identification of such pressure.

As a further example, in some embodiments sensor 112 may include a temperature sensor adapted to identify transition of capsule 101 from an area with ambient temperature (e.g. outside the human body) to an area with a human body temperature and to provide an activation input in response to identification of such a transition.

As another example, in some embodiments sensor 112 may include a motion or acceleration sensor, such as an accelerometer, adapted to identify an activation motion carried out by a user on capsule 101 and to provide an activation input in response to identification of such a transition.

As a further example, in some embodiments sensor 112 may include a moisture sensor adapted to identify transition of capsule 101 from a dry area (e.g. outside the human body) to a moist area (e.g. within the human body) and to provide an activation input in response to identification of such a transition.

In some embodiments, system 100 further includes a control unit 120, which may be remote from capsule 101, and which is adapted to provide one or more inputs to the capsule. In some such embodiments, capsule 101 further includes a remote input receiving mechanism 116, functionally associated with control mechanism 106, and adapted to receive inputs from an input providing mechanism 122 of control unit 120.

In some embodiments, control unit 120 may further include a timing mechanism 126, adapted to track at least one time characteristic, such as a duration that has passed since a control instruction was provided to capsule 101.

In some embodiments, control unit 120 may further include a user input receiver 128, such as a keyboard, touch screen, or touch pad, adapted to receive input from a user, such as the subject, a medical professional treating the subject, or a caregiver of the subject.

Control unit 120 may be any suitable type of control unit. In some embodiments, control unit may be a suitably configured smart phone or a tablet computer.

In some such embodiments, control unit 120 may provide inputs to capsule 101 by remotely transmitting the inputs from input providing mechanism 122 to remote input receiving mechanism 116, for example using a short range wireless communication method, such as radio frequency (RF) communication or Bluetooth® communication. One example of such a mechanism for providing input to a capsule is described in U.S. patent application Ser. No. 15/132,039 filed Apr. 18, 2016 and entitled “IN VIVO DEVICE AND METHOD OF USING THE SAME”, which is incorporated by reference for all purposes as if fully set forth herein.

In some embodiments, control unit 120 is adapted to provide the activation input to control mechanism 106 of capsule 101. In some such embodiments, control unit 120 provides the activation input prior to the subject ingesting capsule 101, whereas in other embodiments control unit 120 provides the activation input following ingestion of capsule 101 by the subject.

Relating to the characteristics of vibrating agitation mechanism 104, the vibrating agitation mechanism may be any suitable mechanism that can be intermittently activated and can apply suitable forces onto capsule housing 102 while maintaining the integrity of the capsule and of the capsule housing.

In some embodiments, intermittently activated vibrating agitation mechanism 104 may include a radial agitation mechanism adapted to exert radial forces on capsule housing 102, in a radial direction with respect to the longitudinal axis of housing 102. For example, the radial agitation mechanism may include an unbalanced weight attached to a shaft of an electric motor powered by said battery, substantially as described in U.S. Pat. No. 9,707,150, which is incorporated by reference for all purposes as if fully set forth herein.

In some embodiments, intermittently activated vibrating agitation mechanism 104 may include an axial agitation mechanism adapted to exert radial forces on the capsule housing 102, in an axial direction with respect to a longitudinal axis of housing 102. For example, the axial agitation mechanism may include an electric motor powered by the battery and an urging mechanism, associated with, and driven by, the electric motor, such that the urging mechanism adapted to exert said axial forces, substantially as described in U.S. Pat. No. 9,707,150. In some embodiments, the urging mechanism adapted to exert the axial forces in opposite directions. In some embodiments, the urging mechanism is adapted to deliver at least a portion of the axial forces in a knocking mode.

In some embodiments, the forces exerted by intermittently activated vibrating agitation mechanism 104 on capsule housing 102 in the vibration mode of operation include radial forces in a radial direction with respect to the longitudinal axis of the housing and axial forces in an axial direction with respect to the longitudinal axis. In some embodiments, a single agitation mechanism exerts both the radial and the axial forces. In other embodiments, the axial forces are exerted by one agitation mechanism, and the radial forces are exerted by another, separate, agitation mechanism, where both agitation mechanisms form part of intermittently activated vibrating agitation mechanism 104.

In some embodiments, the intermittently activated vibrating agitation mechanism 104 may include a magnet mounted onto a rotor adapted to exert a magnetic field as well as radial forces on capsule housing 102. For example, such a magnetic vibration agitation mechanism is described in U.S. patent application Ser. No. 15/058,216 filed on Mar. 2, 2016 and entitled “PHYSIOTHERAPY DEVICE AND METHOD FOR CONTROLLING THE PHYSIOTHERAPY DEVICE”, which is incorporated by reference for all purposes as if fully set forth herein.

In some embodiments, housing 102 may include first and second members, and vibrating agitation mechanism 104 may include a mechanism adapted to effect a vibration by moving the first member of the housing in the opposite direction relative to the second member of the housing, substantially as described in U.S. Pat. No. 9,078,799, which is incorporated by reference for all purposes as if fully set forth herein.

In some embodiments, housing 102 may include a vibration agitation mechanism 104 which makes use of a pendulum to cause vibration in the vicinity of the capsule, for example as described in CN Patent Application Number 105997466 filed on Jun. 16, 2016 and entitled “INTELLIGENT VIBRATING ELECTRONIC CAPSULE”, which is incorporated by reference for all purposes as if fully set forth herein.

In the vibrating mode of operation, intermittently activated vibrating agitation mechanism 104 is adapted to have a plurality of vibration cycles, where each cycle includes a vibration duration followed by a repose duration. Forces are exerted by the vibrating agitation mechanism 104 on capsule housing 102 only during the vibration duration, and as such capsule housing 102 only exerts forces on an environment thereof during the vibration duration.

In some embodiments, the number of vibration cycles per hour is in the range of to 400, 40 to 400, 60 to 400, 80 to 400, 40 to 380, 60 to 380, 80 to 380, 40 to 360, 60 to 360, 80 to 360, 100 to 360, 100 to 330, 100 to 300, 100 to 280, 100 to 250, 100 to 220, 100 to 200, 120 to 300, 120 to 280, 120 to 250, 120 to 220, 120 to 200, 150 to 300, 150 to 280, 150 to 250, 150 to 220, 150 to 200, 170 to 300, 170 to 250, 170 to 220, or 170 to 200.

In some embodiments, the repose duration is greater than the vibration duration.

In some embodiments, the vibration duration is in the range of 0.1 second to 10 seconds, 1 second to 10 seconds, 1 second to 9 seconds, 2 seconds to 9 seconds, 3 seconds to 9 seconds, 3 seconds to 8 seconds, 3 seconds to 7 seconds, 3 seconds to 6 seconds, or 4 seconds to 6 seconds.

In some embodiments, the repose duration is in the range of 1 second to 180 seconds, 3 seconds to 180 seconds, 5 seconds to 180 seconds, 5 seconds to 150 seconds, seconds to 120 seconds, 8 seconds to 100 seconds, 8 seconds to 30 seconds, 10 seconds to 80 seconds, 10 seconds to 70 seconds, 10 seconds to 60 seconds, 10 seconds to 50 seconds, 10 seconds to 40 seconds, 10 seconds to 30 seconds, 10 seconds to 20 seconds, or 15 seconds to 20 seconds.

In some embodiments, the total duration of one vibration cycle is in the range of 1.1 seconds to 200 seconds, 5 seconds to 200 seconds, 10 seconds to 200 seconds, 10 seconds to 150 seconds, 10 seconds to 100 seconds, 10 seconds to 80 seconds, 10 seconds to 50 seconds, 10 seconds to 40 seconds, 10 seconds to 30 seconds, 15 seconds to 50 seconds, 15 seconds to 40 seconds, 15 seconds to 30 seconds, or 15 seconds to 25 seconds.

In some embodiments, the cumulative duration of the vibrating mode of operation, or the cumulative duration during which vibration cycles are occurring, is in the range of 1 hour to 12 hours, 2 hours to 10 hours, 2 hours to 8 hours, 2 hours to 6 hours, 2 hours to 4 hours, or 2 hours to 3 hours. It will be appreciated that the cumulative duration of vibration cycles may be dependent on properties of power source 108.

It will be appreciated by persons skilled in the art that the vibration mode of operation may be intermittent, or interrupted, such that vibrating agitation mechanism 104 is operative in the vibration mode for a first duration, for example 30 minutes, then does have any vibration cycles for a second duration, for example 1 hour, and then is operative in the vibration mode and has vibration cycles for a third duration, for example two hours. The cumulative duration relates to the sum of all durations during which vibrating agitation mechanism 104 was operative in the vibration mode and included vibration cycles, including the vibration duration and the repose duration of the vibration cycle.

In some embodiments, vibrating agitation mechanism 104 is configured to exert forces on the capsule housing 102, such that a net force exerted by the capsule housing 102 on the environment thereof is in the range of 50 grams force (gf) to 600 gf, 50 gf to 550 gf, 100 gf to 550 gf, 100 gf to 500 gf, 150 gf to 500 gf, 200 gf to 500 gf, or 200 gf to 450 gf.

In some embodiments, vibrating agitation mechanism 104 is configured to exert said forces on capsule housing 102 to attain a capsule housing 102 vibrational frequency within a range of 10 Hz to 650 Hz, 15 Hz to 600 Hz, 20 Hz to 550 Hz, 30 Hz to 550 Hz, 50 Hz to 500 Hz, 70 Hz to 500 Hz, 100 Hz to 500 Hz, 130 Hz to 500 Hz, or 150 Hz to 500 Hz.

It will be appreciated that the exact specifications of the capsule, such as the specific frequency and force ranges applicable to a specific capsule, are dependent on the specifications of the power source and of the vibrating agitation mechanism.

It will be further appreciated that a specific capsule may be controlled by the control mechanism such that different vibrational frequencies may be attained and/or different net forces may be exerted, by the capsule in different vibration cycles of the capsule. Due to the natural distinction between subjects, use of multiple different parameters in different vibration cycles of a single capsule would allow the capsule to successfully treat multiple subjects, even if the personal optimal treatment for those subjects is not the same, as there is a higher chance that in at least some of the vibration cycles the activation parameters of the capsule would reach, or be close to, the optimal parameters for each specific subject.

Control mechanism 106 is adapted to control the operation of intermittently activated vibrating agitation mechanism 104. Such control may include control of any one or more of the force applied by the vibrating agitation mechanism, the vibrational frequency reached, the times in which vibrating agitation mechanism 104 operates in the vibration mode of operation, the vibration duration of each vibration cycle, the repose duration of each vibration cycle, the vibration cycle duration, and cumulative vibration duration of the vibrating agitation mechanisms.

In some embodiments, control mechanism 106 is adapted to receive information relating to the desired vibration protocol from control unit 120, prior to ingestion of the capsule or to activation thereof, or during the capsule's traversal of the subject's GI tract. For example, the information may be remotely transmitted from control unit 120 to control mechanism 106, for example using a short range wireless communication method. In some embodiments, the information is transmitted as a list of vibration parameters for effecting the vibration protocol. In some embodiments, the information is transmitted as executable code for effecting the first vibration protocol.

In some embodiments, the information includes a desired number of vibration cycles, a desired vibration duration in each vibration cycle, a desired repose duration in each vibration cycle, a desired cumulative vibration duration, and the like.

Control mechanism 106 may be adapted to control vibrating agitation mechanism 104 so that the capsule applies forces to an environment thereof, such that within the gastrointestinal tract, a mechanical stimulation of the wall of the gastrointestinal tract is effected. As explained in further detail hereinbelow, in some embodiments, control mechanism 106 is adapted to control vibrating agitation mechanism 104 so that the capsule applies forces to an environment thereof. In some embodiments the forces effect a mechanical stimulation of the wall of the gastrointestinal tract of the subject at the predetermined time(s). In some embodiments the forces are applied during at least two distinct time periods. For example, control mechanism 106 may control vibrating agitation mechanism 104 to be in the vibration mode of operation while the capsule is disposed within the stomach of the user, to be in the rest mode of operation while the capsule traverses the small intestine of the user, and to be again in the vibration mode of operation while the capsule is in the large intestine of the user.

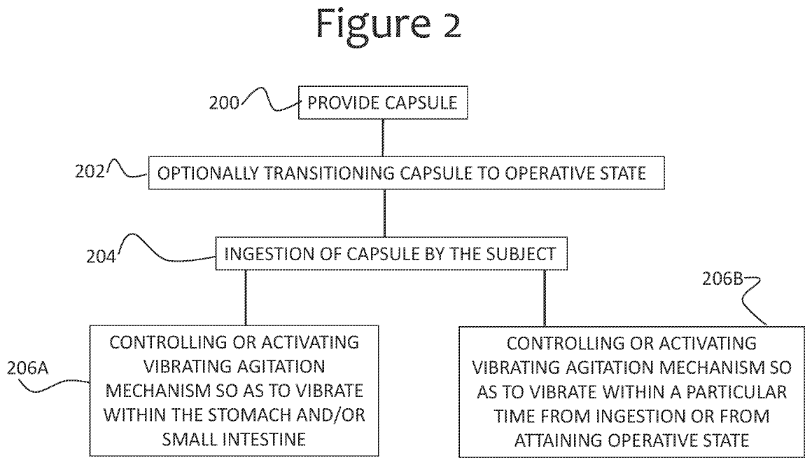

Reference is now additionally made to FIG. 2, which is a schematic flowchart of a method for using a vibrating gastrointestinal capsule in the treatment of Parkinsonism in a subject, according to the present invention, the treatment being based on use of a gastrointestinal treatment system including (or consisting of) a vibrating ingestible capsule, such as capsule 101 of system 100 of FIG. 1.

As seen at step 200, vibrating gastrointestinal capsule is provided. The vibrating gastrointestinal capsule may have, as described with respect to FIG. 1, a housing; a vibrating agitation mechanism adapted such that, in a first vibrating mode of operation, the housing exerts vibrations on an environment surrounding the vibrating gastrointestinal capsule; and a power supply disposed within the housing and adapted to power the vibrating agitation mechanism. Typically, the capsule includes an on-board control mechanism adapted to control or activate the vibrating agitation mechanism. The control mechanism may form a component of such a vibrating agitation mechanism.

At step 204, the vibrating gastrointestinal capsule is ingested by the subject.

As shown, step 206A includes activating or controlling the vibrating agitation mechanism within the capsule such that at least a portion of the first vibrating mode of operation occurs within a portion of a gastrointestinal tract of the subject. This portion consists, at most, of the stomach, small intestine, and large intestine. More typically, however, for the treatment of Parkinsonism, this portion consists, at most, of the stomach and small intestine.

Additionally or alternatively, the vibrating agitation mechanism within the capsule may be activated or controlled (step 206B) such that at least a portion of the first vibrating mode of operation occurs within 6 hours or within 5 hours, more typically within 4.5 hours or within 4 hours, and yet more typically, within 3.5 hours, within 3 hours, within 2.5 hours, within 2 hours, within 1.5 hours, within 1 hour, or within 0.5 hours of the ingesting of the vibrating gastrointestinal capsule, or from attaining the operative state (step 202), as described hereinabove.

This may be done, by way of example, such that the vibrations will be effected within the stomach and/or small intestine, so as to treat the Parkinsonism.

The capsule may be pre-programmed with a vibration protocol. This protocol may include, by way of example, a particular or pre-determined activation time following ingestion, in which the capsule is transitioned from an inoperative state to an operative state. Alternatively or additionally, the capsule may receive an activation input in an active fashion (e.g., from an external controller via RF) or in a passive fashion (e.g., a signal from a sensor to the on-board controller). It will be appreciated that step 202 may be performed after ingestion of the capsule by the subject (e.g., in the case of external control via RF).

In some embodiments, control mechanism 106 may optionally receive a desired vibration protocol for the subject.

In some embodiments, providing of the predetermined time(s) at step 202 and/or providing the desired vibration protocol for the subject at step 204 occurs at the time of manufacturing of the capsule, for example by pre-programming the time into the control mechanism.

In some embodiments, providing of the predetermined time(s) at step 202 and/or providing the desired vibration protocol for the subject at step 204 may be effected by a control unit, such as control unit 120 of FIG. 1.

The programming of the vibration protocol may include remotely transmitting the desired vibration protocol from control unit 120 to control mechanism 106, for example using a short-range wireless communication method. In some embodiments, the desired vibration protocol is transmitted as a list of vibration parameters for effecting the vibration protocol. In some embodiments, the desired vibration protocol is transmitted as executable code for effecting the vibration protocol.

As discussed hereinabove, in some embodiments the activation input may be received from the control unit 120 or from sensors within the capsule sensing that the capsule has been ingested or that a user has carried out an activation motion with the capsule.

Substantially as described hereinabove, the capsule may be activated prior to the user ingesting the capsule at step 204, for example by a signal from the control unit or by the user carrying out an activation motion. In other embodiments, the activation input is provided at the time of ingestion or immediately thereafter, for example by sensors sensing a change in the environment of the capsule due to its ingestion, as described at length hereinabove. In yet other embodiments, the activation input may be provided remotely when the capsule is already in the body of the subject, for example by remote communication from control module 120.

Following activation of capsule 101, or together therewith, capsule 101 is ingested by the subject, and begins to travel through the gastrointestinal tract of the subject, as evident from step 204.

Operation of vibrating agitation mechanism 104 in the vibrating mode of operation at step 206A or 206B effects vibration of capsule housing 102, as described hereinabove, such that the housing exerts vibrations on the environment surrounding the capsule. Specifically, vibration of capsule housing 102 may be intended to effect a mechanical stimulation of the wall of the gastrointestinal tract at the predetermined time of day.

The importance of the gut-brain axis in prevention or improvement of ailments of the GI tract, such as irritable bowel syndrome (IBS) and chronic idiopathic constipation (CIC) has been shown, for example, in “Irritable bowel syndrome, the microbiota and the gut-brain axis” to Raskov et al (published in Gut Microbes. 2016; 7(5): 365-383), which is incorporated by reference as if fully set forth herein. Raskov et al show that the bidirectional neurohumoral integrated communication between the microbiota and the autonomous nervous system, which is called the gut-brain-axis, integrates brain and GI functions, such as gut motility, appetite and weight. Raskov et al further state that “the gut-brain-axis has a central function in the perpetuation of irritable bowel syndrome and the microbiota plays a critical role”.

The connection between ailments of the GI tract, such as IBS and CIC, and Parkinson's disease or Parkinsonism, has been shown, for example in “A Role for Neuronal Alpha-Synuclein in Gastrointestinal Immunity” to Stolzenberg et al (J Innate Immun 2017; 9:456-463), and in “Medical records documentation of constipation preceding Parkinson disease” to Savica et al (Neurology, Nov. 24, 2009; 73(21)), which are both incorporated by reference as if fully set forth herein. According to the findings of Stolzenberg et al and of Savica et al, chronic gastrointestinal ailments may be a trigger, or a precursor, of Parkinson's disease, or of parkinsonism. Such chronic ailments of the GI tract may occur as early as 20 or more years before onset of motor symptoms, and are associated with increased risk of Parkinson's disease.

Without wishing to be bound by theory, Applicants believe that stimulation of the wall of the gastrointestinal tract at step 206A or 206B activates the gut-brain axis to indicate to the brain that gut function is required, thereby causing the brain to trigger the GI tract to better stimulate contractions and peristalsis in the GI tract, and specifically in the large intestine and in the colon. Given the prevalent evidence regarding the association of the gut-brain axis in development and deterioration of Parkinson's disease and its neurodegenerative symptoms, such activation of the gut-brain axis and improvement of GI function improves, and/or delays symptoms of, Parkinson's disease and Parkinsonism.

A treatment session as defined in steps 200 to 206A or 206B may be repeatedly administered to the subject as specified in the treatment protocol for the subject. In some embodiments, the treatment protocol includes administering a plurality of treatment sessions to the subject. In some embodiments, the treatment protocol includes administering at least one treatment session to the subject per week, over a treatment period of at least two weeks, at least at least three weeks, at least four weeks, at least five weeks, at least six weeks, or at least eight weeks. In some embodiments, the treatment protocol includes administering 1 to 7 treatment sessions per week, 3 to 14 treatment sessions per two weeks, 2 to 7 treatment sessions per week, 5 to 14 treatment sessions per two weeks, 3 to 7 treatment sessions per week, 7 to 14 treatment sessions per two weeks, 4 to 7 treatment sessions per week, or 5 to 7 treatment sessions per week.

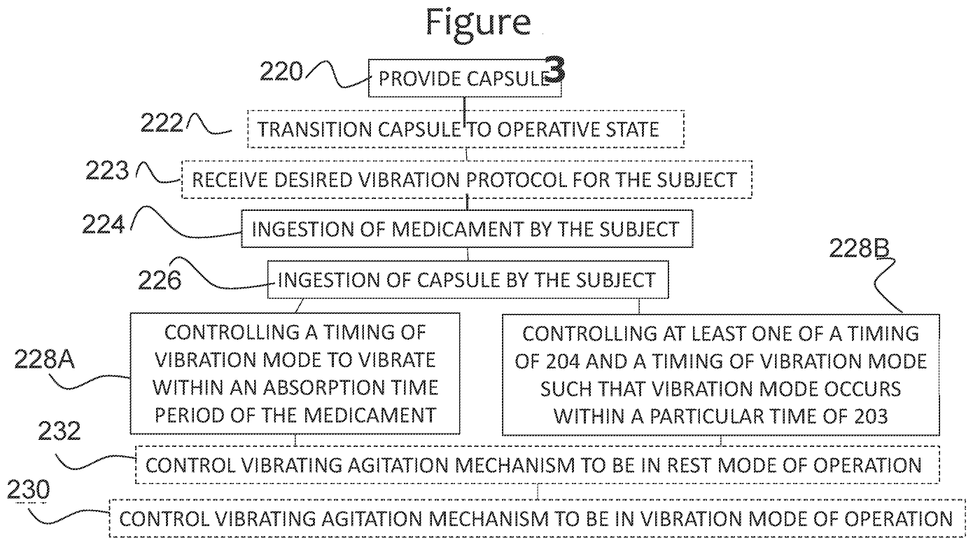

Reference is now additionally made to FIG. 3, which is a schematic flowchart of a method for using a vibrating gastrointestinal capsule to improve or accelerate the absorption into the bloodstream of an ingestible medicament, for example an ingestible medicament for treatment of Parkinsonism or of an ailment of the GI tract, and/or to improve the efficacy of such a medicament, according to the present invention. The method may be based on the use of a gastrointestinal capsule system including (or consisting of) a vibrating ingestible capsule, such as capsule 101 of system 100 of FIG. 1.

As seen at step 220, vibrating gastrointestinal capsule is provided. The vibrating gastrointestinal capsule may have, as described with respect to FIG. 1, a housing; a vibrating agitation mechanism adapted such that, in a first vibrating mode (“vibration mode”) of operation, the housing exerts vibrations on an environment surrounding the vibrating gastrointestinal capsule; and a power supply disposed within the housing and adapted to power the vibrating agitation mechanism. Typically, the capsule includes an on-board control mechanism adapted to control or activate the vibrating agitation mechanism. The control mechanism may form a component of such a vibrating agitation mechanism.

At step 224, an ingestible medicament is ingested by the subject.

In some embodiments, the ingestible medicament may be an ingestible medicament for treatment of Parkinsonism, which may be, or include, any one or more of:
levodopa;
at least one dopaminergic agent;
at least one catecholamine precursor;
a dopamine precursor; at least one dopamine precursor agent, such as (L)-3,4-dihydroxyphenylalanine;
N-methyl-N-(2-propynyl)-2-methyl-1-phenylethyl-2-amine;
tyrosine hydroxylase;
apomorphine;
at least one anticholinergic agent;
at least one agent selected to antagonize at least one cholinergic receptor;
at least one of benzhexol and orphenadrine; and
at least one selective allosteric potentiator of metabotropic glutamate receptor 4 (mGluR4), such as N-phenyl-7-(hydroxylimino)cyclopropa[b]chromen-1a-carboxamide.

In some embodiments, the ingestible medicament may be an ingestible medicament for treatment of an ailment of the GI tract, which may be, or include, at least one of:
at least one osmotic agent, such as magnesium citrate, magnesium hydroxide, polyethylene glycol, or sodium phosphate; MiraLAX®;
at least one contraction stimulating agent, such as bisacodyl or senna;
at least one of Correctol, Ducodyl, Dulcolax, Senexon, and Senokot;
at least one stool softening agent, such as docusate sodium;
Colace;
Linaclotide;
Lactulose;
lubiprostone;
plecanatide;
prucaltride; and
loperamide or bismuth subsalicylate.

In some embodiments, the ingestible medicament may be ingested directly, for example by ingesting a tablet, capsule, liqui-gel capsule, chewable tablet, syrup, or any other form of dosage including the medicament. In such embodiments, the ingestible medicament is considered to be exposed to fluids of the gastrointestinal tract from the moment of ingestion thereof.

In some embodiments, the vibrating ingestible capsule is devoid of a compartment or chamber in which the ingestible medicament may be disposed, for example during ingestion of the capsule.

In some embodiments, the ingestible medicament may be ingested within a medicament delivery capsule, adapted to deliver the medicament to a specific location in the gastrointestinal tract. In such embodiments, the ingestible medicament is considered to be exposed to fluids of the gastrointestinal tract from a time when the fluids of the gastrointestinal tract an enter a portion of the medicament delivery capsule holding the ingestible medicament, or from a time that the ingestible medicament is released from the medicament delivery capsule into the gastrointestinal tract.

Examples of such medicament delivery capsules are described in:
U.S. Pat. Nos. 5,170,801; 6,632,216; 6,776,165; 6,929,363; 8,202,697; 8,518,022; 8,597,278; and 8,771,730;
U.S. Patent Application Publication Numbers 2004/0253304; 2004/0267240; 2005/0058701; 2005/0148847; 2008/0275430; 2009/0306633; 2010/0049012; and 2016/0136104.
PCT Patent Application Publication Numbers WO2006/025013; WO2008/012700; and WO 2009/063375;
all of which are incorporated by reference for all purposes as if fully set forth herein.

At step 226, the vibrating gastrointestinal capsule is ingested by the subject.

While the ingestible medicament and the vibrating gastrointestinal capsule may be ingested at the same time, or within 0-30 minutes of each other, no order of action is implied by FIG. 3, and the capsule may be ingested prior to the medicament.

In some embodiments, the vibrating gastrointestinal capsule functions also as a medicament delivery system for delivery of the ingestible medicament and delivers the medicament to a specific area or location in the gastrointestinal tract of the subject. In such embodiments, the subject ingests a single capsule, the vibrating ingestible capsule, thereby fulfilling steps 224 and 226 of the method.

As shown, step 228A includes controlling at least one of a time of ingesting the vibrating gastrointestinal capsule and a timing of said vibration mode of operation (e.g., when the vibration mode is initiated, a duration of the vibration mode, etc.) to at least partially transpire within at an absorption time period of the ingestible medicament within the gastrointestinal tract of the subject. Typically, the absorption time period is an estimated absorption time period, as defined herein. In some cases, an actual absorption time period may be determined, again, as defined herein.

Additionally or alternatively, the method may include controlling at least one of a timing of ingesting the vibrating gastrointestinal capsule and a timing of said vibration mode of operation such that the vibration mode at least partially transpires within a particular time period with respect to the ingesting of the ingestible medicament (step 228B).

The particular time period may be within 5 hours, within 4 hours, within 3.5 hours, within 3 hours, or within 2.5 hours of the ingesting of the ingestible medicament, and more typically, within 2 hours, within 1.5 hours, within 1 hour, or within 0.5 hours thereof.

In some embodiments, the method may include controlling at least one of a timing of ingesting the vibrating gastrointestinal capsule and a timing of said vibration mode of operation such that said vibration mode at least partially transpires when the capsule is in a region of the gastrointestinal tract in which the medicament is typically absorbed into the bloodstream. The region of the gastrointestinal tract may include one or more of the stomach of the subject, the duodenum of the subject, the small intestine of the subject, the large intestine of the subject, or the colon of the subject.

For example, when cooperating with the medicament levadopa (for treatment of Parkinsonism), which is typically absorbed into the bloodstream through the stomach walls and/or the small intestine walls, the vibration mode at least partially transpires within a time period in which the capsule traverses, or is expected to traverse, the stomach and small intestine.

As another example, when cooperating with the medicament Linzess® (for treatment of constipation), which is typically absorbed into the bloodstream through the stomach walls and/or the small intestine walls, the vibration mode at least partially transpires within a time period in which the capsule traverses, or is expected to traverse, the stomach and small intestine.

In some embodiments, and as described in further detail herein, the method may include transitioning the capsule (from an inoperative state) to an operative state.

The capsule may be pre-programmed with a vibration protocol. This protocol may include, by way of example, a particular or pre-determined activation time following ingestion, in which the capsule is transitioned from an inoperative state to an operative state.

Alternatively or additionally, the capsule may receive an activation input in an active fashion (e.g., from an external controller via RF) or in a passive fashion (e.g., a signal from a sensor to the on-board controller). It will be appreciated that step 222, in which the vibrating ingestible capsule is transitioned from the inoperative state to the operative state, may be performed after ingestion of the capsule by the subject (e.g., in the case of external control via RF).

In some embodiments, control mechanism 106 may optionally receive a desired vibration protocol for the subject, at an optional step 223.

In some embodiments, providing of the predetermined time(s) at step 222 and/or providing the desired vibration protocol for the subject at step 223 occurs at the time of manufacturing of the capsule, for example by pre-programming the time into the control mechanism.

In some embodiments, providing of the predetermined time(s) at step 222 and/or providing the desired vibration protocol for the subject at step 223 may be effected by a control unit, such as control unit 120 of FIG. 1.

The programming of the vibration protocol may include remotely transmitting the desired vibration protocol from control unit 120 to control mechanism 106, for example using a short-range wireless communication method. In some embodiments, the desired vibration protocol is transmitted as a list of vibration parameters for effecting the vibration protocol. In some embodiments, the desired vibration protocol is transmitted as executable code for effecting the vibration protocol.

As discussed hereinabove, in some embodiments the activation input may be received from the control unit 120 or from sensors within the capsule sensing that the capsule has been ingested or that a user has carried out an activation motion with the capsule.

Substantially as described hereinabove, the vibrating ingestible capsule may be activated prior to the user ingesting the capsule at step 226, for example by a signal from the control unit or by the user carrying out an activation motion. In other embodiments, the activation input is provided at the time of ingestion or immediately thereafter, for example by sensors sensing a change in the environment of the capsule due to its ingestion, as described at length hereinabove. In yet other embodiments, the activation input may be provided remotely when the capsule is already in the body of the subject, for example by remote communication from control module 120.

Following activation of capsule 101, or together therewith, capsule 101 is ingested by the subject, and begins to travel through the gastrointestinal tract of the subject, as evident from step 226.

Operation of vibrating agitation mechanism 104 in the vibrating mode of operation at step 228A or 228B effects vibration of capsule housing 102, as described hereinabove, such that the housing exerts vibrations on the environment surrounding the capsule. The exerted vibration maintains the integrity of capsule housing 102 and of capsule 101. Specifically, vibration of capsule housing 102 may be intended to effect a mechanical stimulation of the wall of the gastrointestinal tract at a time when the ingested medicament may be absorbed, thereby to increase or accelerate the absorption of the ingested medicament into the bloodstream of the subject, for delivery to a target treatment region of the medicament.

For example, the target treatment region of medicaments for treatment of Parkinsonism is in or near the brain, and as such an ingestible medicament for treatment of Parkinsonism, when absorbed to the bloodstream in the gastrointestinal tract, is delivered to the target treatment region away from the gastrointestinal tract.

In some embodiments, an additional step 230 includes controlling the vibrating agitation mechanism of the capsule 101 to also operate in the vibration mode of operation also following the absorption time period of the ingestible medicament, and/or further along the GI tract from the region in which the ingestible medicament is typically absorbed. For example, step 228A may occur when the capsule 101 is located in the stomach of the user, where the ingestible medicament is typically absorbed, and step 230 may occur when capsule 101 is located in the colon of the user, for example to ease constipation symptoms of the user.

In some embodiments, between steps 228A or 218B, in which the capsule is in the vibration mode of operation, and step 230 in which the capsule is again in the vibration mode of operation, control mechanism 106 may control capsule 101 to be in the rest mode of operation at optional step 232. Relating back to the example above, during the time the capsule travels from the stomach of the subject (where it is in the vibration mode of operation—step 228A) to the colon of the subject (where it is in the vibration mode of operation—step 230), the control mechanism controls the capsule to be in the rest mode of operation.

A treatment session as defined in FIG. 3 may be repeatedly administered to the subject as specified in the treatment protocol for the subject. In some embodiments, the treatment protocol includes administering a plurality of treatment sessions to the subject. In some embodiments, the treatment protocol includes administering at least one treatment session to the subject per week, over a treatment period of at least two weeks, at least at least three weeks, at least four weeks, at least five weeks, at least six weeks, or at least eight weeks. In some embodiments, the treatment protocol includes administering 1 to 7 treatment sessions per week, 3 to 14 treatment sessions per two weeks, 2 to 7 treatment sessions per week, 5 to 14 treatment sessions per two weeks, 3 to 7 treatment sessions per week, 7 to 14 treatment sessions per two weeks, 4 to 7 treatment sessions per week, or 5 to 7 treatment sessions per week.

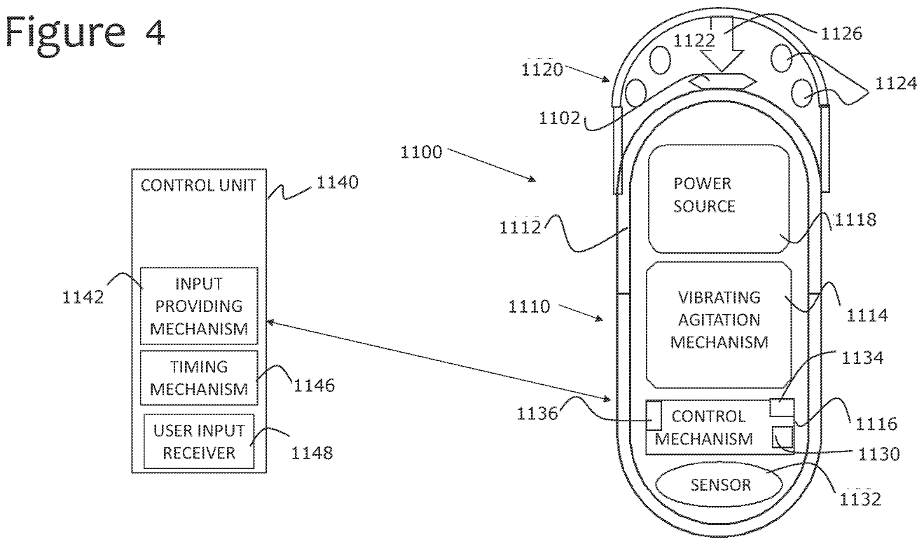

Reference is now made to FIG. 4, which is a schematic block diagram of a device 1100 for delivering an ingestible medicament within a medicament tablet 1102 into the gastrointestinal tract of a user according to an embodiment of the present invention.

As seen in FIG. 4, device 1100 includes vibrating ingestible capsule 1110. Capsule 1110 includes a capsule housing or shell 1112, arranged along a longitudinal axis 1113 and having disposed therein a vibrating agitator 1114. A control element 1116 is adapted to control operation of vibrating agitator 1114, and at least one power source 1118 provides power to vibrating agitator 1114 and control element 1116.

A hollow medicament compartment housing 1120 is associated with capsule housing 1112. Medicament compartment housing 1120 defines a hollow 1122 dimensioned and configured to have medicament tablet 1102 disposed therein. At least one aperture 1124 is formed in medicament compartment housing 1120, the aperture being dimensioned and configured to enable fluid communication between an environment surrounding medicament compartment housing 1120 and hollow 1122.

In some embodiments, apertures 1124 are dimensioned and configured such that, when medicament tablet 1102 is disposed within hollow 1122 and device 1100 is in the gastrointestinal tract of the user, the ingestible medicament of medicament tablet 1102 enters the environment surrounding medicament compartment housing 1120 for delivery thereof to the body of the user.

In some embodiments, medicament compartment housing 1120 also includes at least one biasing mechanism 1126 adapted, when medicament tablet 1102 is disposed within hollow 1122, to bias medicament tablet 1102 toward capsule housing 1112 of capsule 1110. As described hereinbelow, in some embodiments, biasing mechanism 1126 comprises a spring or spring-like element. In some embodiments, biasing mechanism 1126 comprises a pre-loaded leaf, or other pre-loaded element. In some embodiments, biasing mechanism 1126 comprises a plunger, or pre-loaded plunger. In some embodiments, biasing mechanism 1126 comprises a cam.

Relating specifically to capsule 1110, power source 1118 may be any suitable power source, such as one or more alkaline or silver oxide batteries, primary batteries, rechargeable batteries, capacitors and/or supercapacitors.

Intermittently activated vibrating agitator 1114 is adapted to have a vibration mode of operation (also termed the first mode of operation) and a rest mode of operation (also termed the second mode of operation). In the vibration mode of operation, intermittently activated vibrating agitator 1114 is adapted to exert forces on capsule housing 1112, such that capsule housing 1112 exerts vibrations on an environment surrounding capsule 1110 and/or device 1100.

In some embodiments, the capsule 1110 is in an inoperative state, until the receipt of an activation input, which causes control element 1116 to transition the capsule from the inoperative state to an operative state.

In some embodiments, control element 1116 is functionally associated with, or includes, a timing mechanism 1130, powered by power source 1118 and adapted to track at least one time characteristic, such as a duration that has passed since an activation input was received, or a duration that has passed since the user ingested capsule 1110. In some embodiments, the timing mechanism 1130 is a clock. In some embodiments, the timing mechanism 1130 is a timer adapted to measure time relative to a starting point or to “count down” relative to a desired ending point.

In some embodiments, capsule 1110 is devoid of any sensors for sensing an environment thereof. In some such embodiments, control element 1116 is adapted, in response to receipt of an activation input, to wait a predetermined delay time, and following the predetermined delay time, to activate vibrating agitator 1114 to operate in said first vibration mode of operation.

In other embodiments, such as the embodiment illustrated in FIG. 4, capsule 1110 further includes at least one sensor 1132, functionally associated with control element 1116. The at least one sensor 1132 may be adapted to sense at least one parameter within capsule 1110 or in an environment of capsule 1110, and may include a temperature sensor, an illumination sensor, a moisture sensor, a pressure sensor, an accelerometer, or any other suitable sensor. In some embodiments, the at least one sensor 1132 is adapted to identify a specific condition in capsule 1110 or in the vicinity thereof, and to provide an activation input to control element 1116 in response to identification of the condition. For example, in some embodiments the condition is indicative of the user ingesting capsule 1110.

For example, in some embodiments sensor 1132 may include an illumination sensor, adapted to identify transition of capsule 1110 from an illuminated environment (e.g. outside the human body) to a dark environment (e.g. within the human body) and to provide an activation input in response to identification of such a transition.

As another example, in some embodiments sensor 1132 may include a motion or acceleration sensor, such as an accelerometer, adapted to identify an activation motion carried out by a user on capsule 1110 or on device 1100 and to provide an activation input in response to identification of such a transition. An example of an accelerometer providing an activation input for a gastrointestinal capsule is provided in U.S. Pat. No. 10,314,514, which is incorporated by reference for all purposes as if fully set forth herein.

As another example, in some embodiments sensor 1132 may include a pressure sensor adapted identify pressure applied to the capsule 1110 or to device 1100, which pressure is indicative of the capsule moving through a pharynx of the user, and to provide an activation input in response to identification of such pressure.

As a further example, in some embodiments sensor 1132 may include a temperature sensor adapted to identify transition of capsule 1110 or of device 1100 from an area with ambient temperature (e.g. outside the human body) to an area with a human body temperature and to provide an activation input in response to identification of such a transition.

As a further example, in some embodiments sensor 1132 may include a moisture sensor adapted to identify transition of capsule 1110 or of device 1100 from a dry area (e.g. outside the human body) to a moist area (e.g. within the human body) and to provide an activation input in response to identification of such a transition.

It will be appreciated by people of skill in the art that sensor 1132 need not necessarily be disposed within capsule 1110, as illustrated in FIG. 4, and may be disposed anywhere within device 1100, for example within hollow 1122 of medicament compartment housing 1120, on an exterior of capsule 1110, or on the exterior of device 1100.

In some embodiments, device 1100 may be functionally associated with a control unit 1140, which may be remote from device 1100 and from capsule 1110, and which is adapted to provide one or more inputs to the capsule. In some such embodiments, capsule 1110 further includes a remote input receiving mechanism 1136, functionally associated with control element 1116, and adapted to receive inputs from an input providing mechanism 1142 of control unit 1140.

In some embodiments, control unit 1140 may further include a timing mechanism 1146, adapted to track at least one time characteristic, such as a duration that has passed since a control instruction was provided to capsule 1110.

In some embodiments, control unit 1140 may further include a user input receiver 1148, such as a keyboard, touch screen, or touch pad, adapted to receive input from a user, such as the user, a medical professional treating the user, or a caregiver of the user.

Control unit 1140 may be any suitable type of control unit. In some embodiments, control unit may be a suitably configured smart phone or a tablet computer.

In some such embodiments, control unit 1140 may provide inputs to capsule 1110 by remotely transmitting the inputs from input providing mechanism 1142 to remote input receiving mechanism 1136, for example using a short range wireless communication method, such as radio frequency (RF) communication or Bluetooth® communication. One example of such a mechanism for providing input to a capsule is described in U.S. Pat. No. 10,478,373, which is incorporated by reference for all purposes as if fully set forth herein.

In some embodiments, control unit 1140 is adapted to provide the activation input to control element 1116 of capsule 1110. In some such embodiments, control unit 1140 provides the activation input prior to the user ingesting device 1100 including capsule 1110, whereas in other embodiments control unit 1140 provides the activation input following ingestion of device 1100 and capsule 1110 by the user.

Relating to the characteristics of vibrating agitator 1114, the vibrating agitator may be any suitable mechanism that can be intermittently activated and can apply suitable forces onto capsule housing 1112.

In some embodiments, intermittently activated vibrating agitator 1114 may include a radial agitator adapted to exert radial forces on capsule housing 1112, in a radial direction with respect to the longitudinal axis of housing 1112. For example, the radial agitator may include an unbalanced weight attached to a shaft of an electric motor powered by said battery, substantially as described in U.S. Pat. No. 9,707,150, which is incorporated by reference for all purposes as if fully set forth herein.

In some embodiments, intermittently activated vibrating agitator 1114 may include an axial agitator adapted to exert radial forces on the capsule housing 1112, in an axial direction with respect to a longitudinal axis of housing 1112. For example, the axial agitator may include an electric motor powered by the battery and an urging mechanism, associated with, and driven by, the electric motor, such that the urging mechanism adapted to exert said axial forces, substantially as described in U.S. Pat. No. 9,707,150. In some embodiments, the urging mechanism adapted to exert the axial forces in opposite directions. In some embodiments, the urging mechanism is adapted to deliver at least a portion of the axial forces in a knocking mode.

In some embodiments, the forces exerted by intermittently activated vibrating agitator 1114 on capsule housing 1112 in the vibration mode of operation include radial forces in a radial direction with respect to the longitudinal axis of the housing and axial forces in an axial direction with respect to the longitudinal axis. In some embodiments, a single agitator exerts both the radial and the axial forces. In other embodiments, the axial forces are exerted by one agitator, and the radial forces are exerted by another, separate, agitator, where both agitators form part of intermittently activated vibrating agitator 1114.

In some embodiments, the intermittently activated vibrating agitator 1114 may include a magnet mounted onto a rotor adapted to exert a magnetic field as well as radial forces on capsule housing 1112. For example, such a magnetic vibrating agitator is described in US Patent Application Publication No. 2016/0310357, which is incorporated by reference for all purposes as if fully set forth herein.

In some embodiments, housing 1112 may include first and second members, and vibrating agitator 1114 may include an agitator adapted to effect a vibration by moving the first member of the housing in the opposite direction relative to the second member of the housing, substantially as described in U.S. Pat. No. 9,078,799, which is incorporated by reference for all purposes as if fully set forth herein.

In some embodiments, housing 1112 may include a vibrating agitator 1114 which makes use of a pendulum to cause vibration in the vicinity of the capsule, for example as described in CN Patent Application Number 105997466 filed on Jun. 16, 2016, which is incorporated by reference for all purposes as if fully set forth herein.

In some embodiments, or at some times, control element 1116 is adapted to control vibrating agitator 1114, and specifically to set at least one vibration parameter of vibrating agitator 1114, so as to promote absorption of the ingestible medicament into the bloodstream of the user. For example, absorption of the ingestible medicament may be promoted by the vibration breaking down the medicament tablet to smaller, more absorbable parts. As another example, absorption of the ingestible medicament may be promoted by the vibration promoting emulsification of the ingestible medicament. As yet another example, absorption of the ingestible medicament may be promoted by the vibration causing a hydrophobic phase of the ingestible medicament to form smaller bubbles, thereby increasing the surface area of the hydrophobic phase for absorption thereof. In another example, absorption of the ingestible medicament may be promoted by the vibration causing greater exposure of the ingestible medicament to the environment.

In some embodiments, or at some times, control element 1116 may be adapted to control vibrating agitator 1114 so that the capsule applies forces to an environment thereof, such that within the gastrointestinal tract, a mechanical stimulation of the wall of the gastrointestinal tract is effected.

In some such embodiments, the at least one vibration parameter includes at least one of a vibration frequency, a cumulative vibration duration, a number of vibration cycles per time unit, a duration of a vibration duration within a vibration cycle, a duration of a repose duration within a vibration cycle, a total duration of a single vibration cycle, and a net force exerted by said housing on said environment, as explained in further detail hereinbelow.

In some embodiments, control element 1116 is adapted to control a timing or activation delay of the vibration mode of operation of the vibrating agitator 1114 such that the vibration mode of operation at least partially transpires within an estimated absorption time period of the ingestible medicament released from medicament tablet 1102 within the gastrointestinal tract of the user.

In some embodiments, control element 1116 is adapted to control a timing or activation delay of the vibration mode of operation of the vibrating agitator 1114 such that the vibration mode of operation at least partially transpires within an actual absorption time period of the ingestible medicament released from medicament tablet 1102 within the gastrointestinal tract of the user.

In the vibrating mode of operation, intermittently activated vibrating agitator 1114 is adapted to have a plurality of vibration cycles, where each cycle includes a vibration duration followed by a repose duration. Forces are exerted by the vibrating agitator 1114 on capsule housing 1112 only during the vibration duration, and as such, capsule housing 1112 only exerts forces on an environment thereof during the vibration duration.

In some embodiments, the number of vibration cycles per hour is in the range of to 400, 40 to 400, 60 to 400, 80 to 400, 40 to 380, 60 to 380, 80 to 380, 40 to 360, 60 to 360, 80 to 360, 100 to 360, 100 to 330, 100 to 300, 100 to 280, 100 to 250, 100 to 220, 100 to 200, 120 to 300, 120 to 280, 120 to 250, 120 to 220, 120 to 200, 150 to 300, 150 to 280, 150 to 250, 150 to 220, 150 to 200, 170 to 300, 170 to 250, 170 to 220, or 170 to 200.

In some embodiments, the repose duration is greater than the vibration duration.

In some embodiments, the vibration duration is in the range of 0.1 second to 10 seconds, 1 second to 10 seconds, 1 second to 9 seconds, 2 seconds to 9 seconds, 3 seconds to 9 seconds, 3 seconds to 8 seconds, 3 seconds to 7 seconds, 3 seconds to 6 seconds, 4 seconds to 6 seconds, or 5 seconds to 6 seconds.

In some embodiments, the repose duration is in the range of 1 second to 180 seconds, 3 seconds to 180 seconds, 5 seconds to 180 seconds, 5 seconds to 150 seconds, seconds to 120 seconds, 8 seconds to 100 seconds, 8 seconds to 30 seconds, 10 seconds to 80 seconds, 10 seconds to 70 seconds, 10 seconds to 60 seconds, 10 seconds to 50 seconds, 10 seconds to 40 seconds, 10 seconds to 30 seconds, 10 seconds to 20 seconds, or 15 seconds to 20 seconds.

In some embodiments, the total duration of one vibration cycle is in the range of 1.1 seconds to 200 seconds, 5 seconds to 200 seconds, 10 seconds to 200 seconds, 10 seconds to 150 seconds, 10 seconds to 100 seconds, 10 seconds to 80 seconds, 10 seconds to 50 seconds, 10 seconds to 40 seconds, 10 seconds to 30 seconds, 15 seconds to 50 seconds, 15 seconds to 40 seconds, 15 seconds to 30 seconds, or 15 seconds to 25 seconds.

In some embodiments, the cumulative duration of the vibrating mode of operation, or the cumulative duration during which vibration cycles are occurring, is in the range of 1 hour to 12 hours, 2 hours to 10 hours, 2 hours to 8 hours, 2 hours to 6 hours, 2 hours to 4 hours, or 2 hours to 3 hours. It will be appreciated that the cumulative duration of vibration cycles may be dependent on properties of power source 1118.

It will be appreciated by persons skilled in the art that the vibration mode of operation may be intermittent, or interrupted, such that vibrating agitator 1114 is operative in the vibration mode for a first duration, for example 30 minutes, then does have any vibration cycles for a second duration, for example 1 hour, and then is operative in the vibration mode and has vibration cycles for a third duration, for example two hours. The cumulative duration relates to the sum of all durations during which vibrating agitator 1114 was operative in the vibration mode and included vibration cycles, including the vibration duration and the repose duration of the vibration cycle.

In some embodiments, vibrating agitator 1114 is configured to exert forces on the capsule housing 1112, such that a net force exerted by the capsule housing 1112 on the environment thereof is in the range of 50 grams force (gf) to 600 gf, 50 gf to 550 gf, 100 gf to 550 gf, 100 gf to 500 gf, 150 gf to 500 gf, 200 gf to 500 gf, or 200 gf to 450 gf.

In some embodiments, vibrating agitator 1114 is configured to exert said forces on capsule housing 1112 to attain a capsule housing 1112 vibrational frequency within a range of 10 Hz to 650 Hz, 15 Hz to 600 Hz, 20 Hz to 550 Hz, 30 Hz to 550 Hz, 50 Hz to 500 Hz, 70 Hz to 500 Hz, 100 Hz to 500 Hz, 130 Hz to 500 Hz, or 150 Hz to 500 Hz.

It will be appreciated that the exact specifications of the capsule, such as the specific frequency and force ranges applicable to a specific capsule, are dependent on the specifications of the power source 1118 and of the vibrating agitator 1114.

It will be further appreciated that a specific capsule may be controlled by the control element 1116 such that different vibrational frequencies may be attained and/or different net forces may be exerted, by the capsule in different vibration cycles of the capsule. Due to the natural distinction between users, use of multiple different parameters in different vibration cycles of a single capsule would allow the capsule to successfully treat multiple users, even if the personal optimal treatment for those users is not the same, as there is a higher chance that in at least some of the vibration cycles the activation parameters of the capsule would reach, or be close to, the optimal parameters for each specific user.

Control element 1116 is adapted to control the operation of intermittently activated vibrating agitator 1114. Such control may include control of any one or more of the force applied by the vibrating agitator 1114, the vibrational frequency reached, the times in which vibrating agitator 1114 operates in the vibration mode of operation, the vibration duration of each vibration cycle, the repose duration of each vibration cycle, the vibration cycle duration, and cumulative vibration duration of the vibrating agitators.

In some embodiments, control element 1116 is adapted to receive information relating to the desired vibration protocol from control unit 1140, prior to ingestion of device 1100 and capsule 1110 or to activation of the capsule, or during the device's and capsule's traversal of the user's GI tract. For example, the information may be remotely transmitted from control unit 1140 to control element 1116, for example using a short range wireless communication method. In some embodiments, the information is transmitted as a list of vibration parameters for effecting the vibration protocol. In some embodiments, the information is transmitted as executable code for effecting the first vibration protocol.

In some embodiments, the information includes a desired number of vibration cycles, a desired vibration duration in each vibration cycle, a desired repose duration in each vibration cycle, a desired cumulative vibration duration, and the like.

Relating to hollow medicament compartment housing 1120, in some embodiments, medicament compartment housing 1120 is biodegradable. In some embodiments, medicament compartment housing 1120 is digestible by the gastrointestinal tract of the user. In some embodiments, medicament compartment housing 1120 is flexible.

In some embodiments, hollow 1122 has a volume in the range of 200 mm³ to 800 mm³, 300 mm³ to 700 mm³, 400 mm³ to 600 mm³, or 500 mm³.

In some embodiments, apertures 1124 are dimensioned and configured such that medicament tablet 1102, while whole, cannot be removed from hollow 1122 of medicament compartment housing 1120.

In other embodiments, apertures 1124 are dimensioned and configured to enable insertion of medicament tablet 1102, via at least one aperture, into hollow 1122 of medicament compartment housing 1120.

Ins some embodiments, in use during transition of the device 1100 through the gastrointestinal tract of the user, biasing mechanism 1126 is adapted to continue biasing medicament tablet 1102 toward housing 1112 of vibrating ingestible capsule 1110, while the ingestible medicament from medicament tablet 1102 is delivered to the environment surrounding hollow 1122 and device 1100.

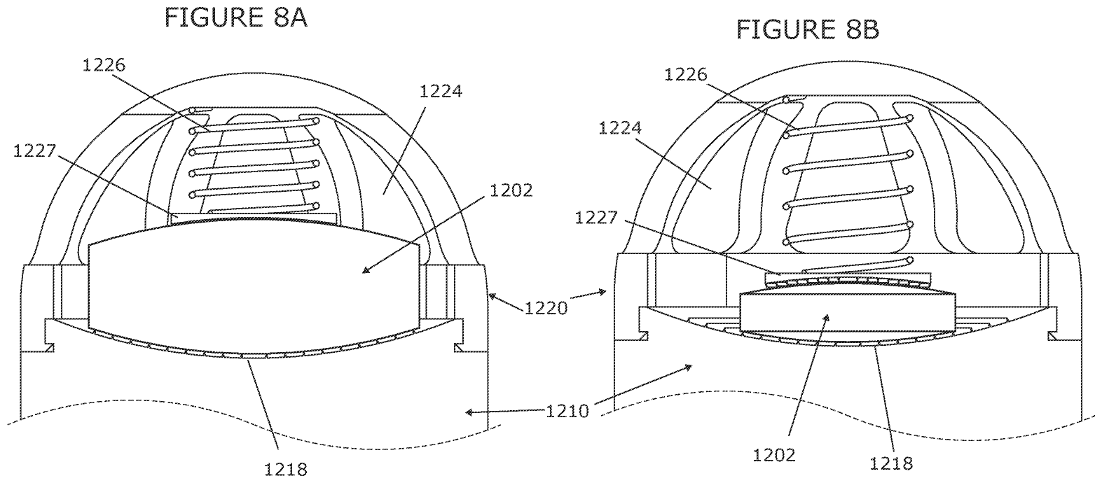

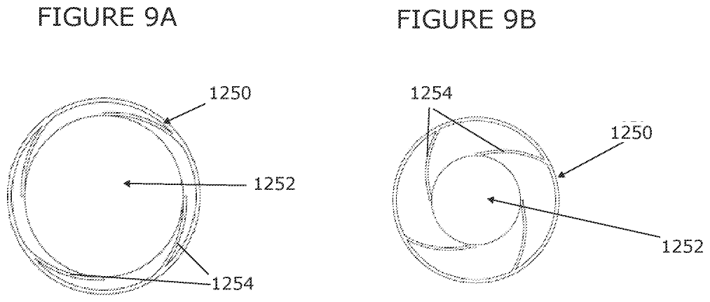

In some embodiments, biasing mechanism 1126 includes at least one pre-loaded compression spring, for example as illustrated in FIGS. 8A and 8B, described in detail hereinbelow. In some embodiments, biasing mechanism 1126 includes at least one flexible and resilient leaf, for example as illustrated in FIGS. 9A and 9B, described in detail hereinbelow.

In some embodiments, biasing mechanism 1126 includes at least one longitudinal biasing mechanism adapted to bias medicament tablet 1102 by application of pressure along a longitudinal axis of the medicament tablet, for example as illustrated in FIGS. 8A and 8B described in detail hereinbelow.

In some embodiments, biasing mechanism 1126 includes at least one radial biasing mechanism adapted to bias medicament tablet 1102 by application of radial pressure along a perimeter of the medicament tablet, for example as illustrated in FIGS. 9A and 9B described in detail hereinbelow.

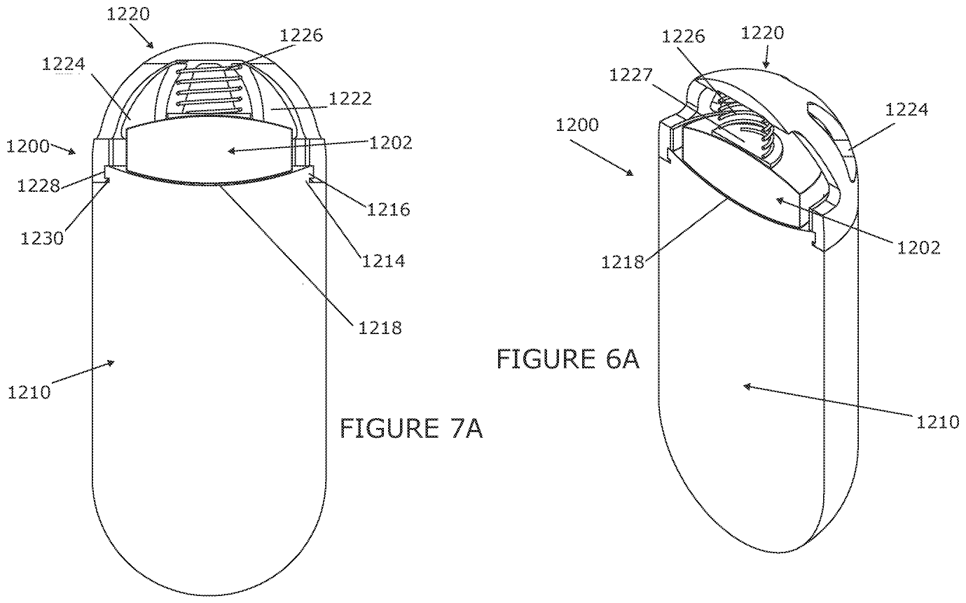

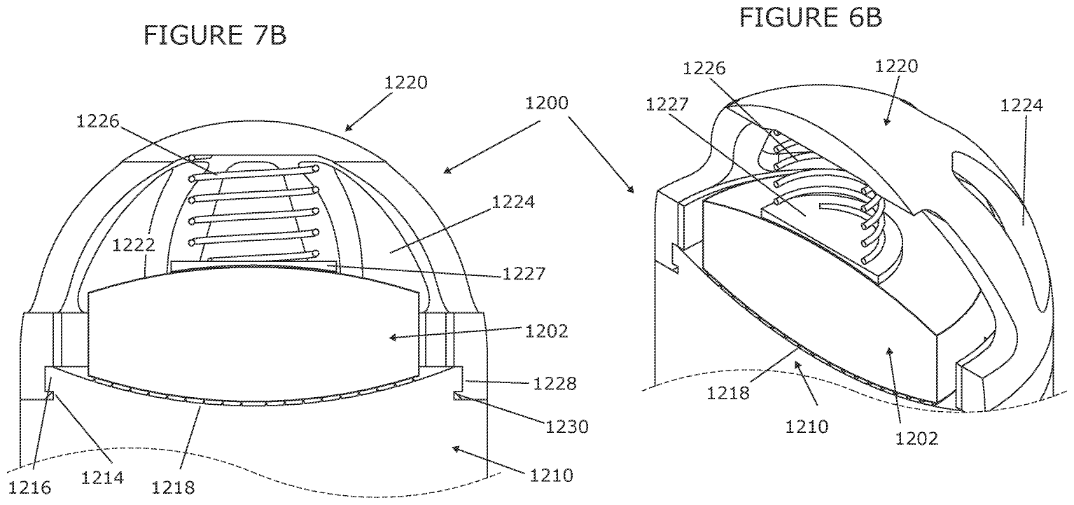

In some embodiments, an exterior contour of at least a portion of housing 1112 of the vibrating ingestible capsule 1110 is adapted to match an exterior contour of the medicament tablet 1102, for example as illustrated in FIGS. 6B and 7B described in detail hereinbelow. In some such embodiments, the exterior contour of at least a portion of housing 1112 is concave as illustrated in FIG. 6B.

In some embodiments, medicament compartment housing 1120 is at least partially attached to housing 1112 of vibrating ingestible capsule 1110. In some embodiments, medicament compartment housing 1120 is fully attached to housing 1112 of vibrating ingestible capsule 1110.

In some embodiments, medicament compartment housing 1120 is fixedly attached to housing 1112 of vibrating ingestible capsule 1110, for example by adhering, by soldering, or by snap fit engagement. In other embodiments, medicament compartment housing 1120 is removably attached to housing 1112 of vibrating ingestible capsule 1110, for example by threaded engagement.

In some embodiments, housing 1112 of vibrating ingestible capsule 1110 includes an attachment mechanism, and medicament compartment housing 1120 includes a corresponding attachment mechanism, for mutual attachment of vibrating ingestible capsule 1110 to hollow medicament compartment housing 1120. An exemplary arrangement of such attachment mechanisms is illustrated, for example, in FIGS. 6B and 7B described in detail hereinbelow.

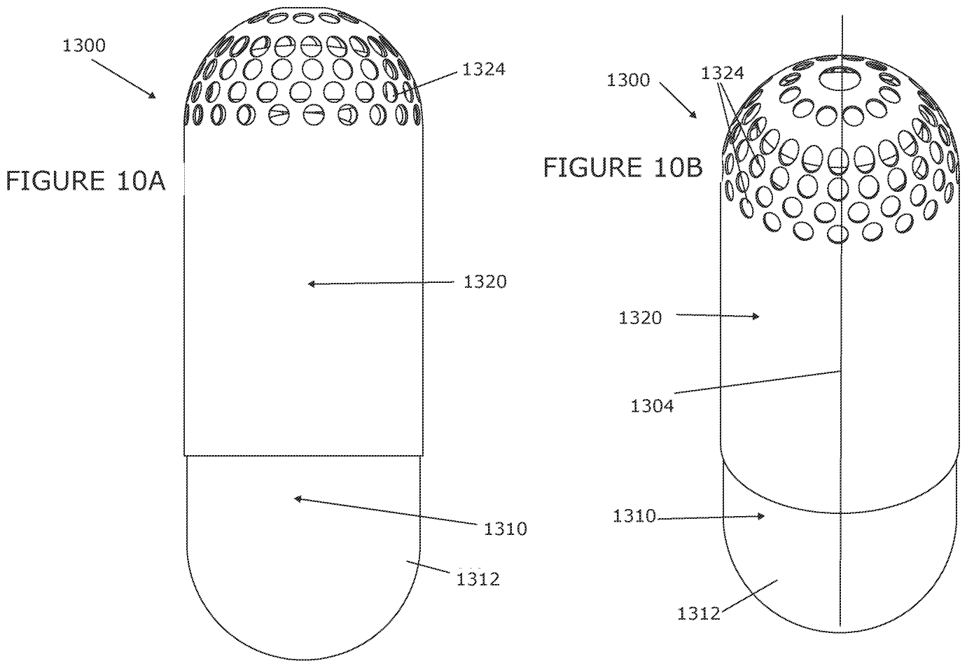

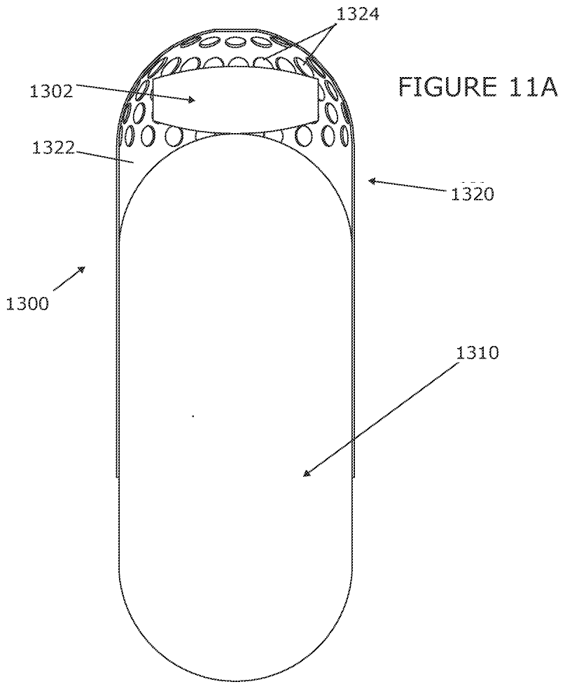

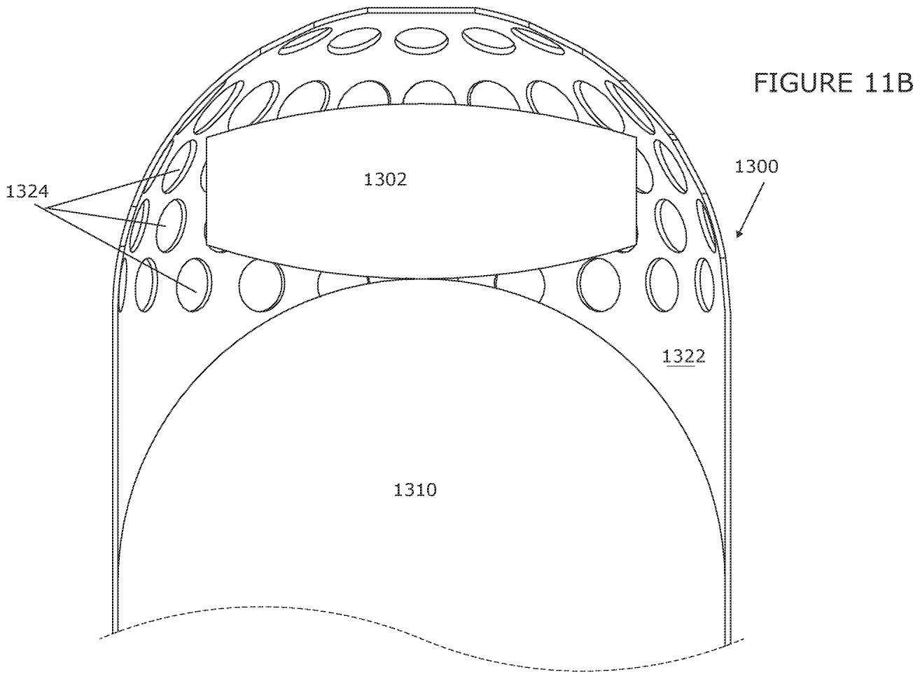

In some embodiments, medicament compartment housing 1120 at least partially envelops housing 1112 of vibrating ingestible capsule 1110, as illustrated, for example, in FIGS. 10A to 11B.

In some embodiments, medicament compartment housing 1120 fully envelops housing 1112 of vibrating ingestible capsule 1110. In some such embodiments, medicament compartment housing 1120 includes a hollow capsule including aperture(s) 1124, which has greater length than the length of vibrating ingestible capsule 1110, and a greater diameter than a diameter of vibrating ingestible capsule. In such embodiments, vibrating ingestible capsule 1110 is disposed within the hollow capsule of the medicament compartment housing.

Turning now to characteristics of the medicament tablet 1102, in some embodiments, medicament tablet 1102 has a diameter of up to 5 mm, up to 6 mm, up to 7 mm, up to 8 mm, or up to 9 mm.

In some embodiments, medicament tablet 1102 has a maximal linear dimension, in any direction of the tablet, of up to 10 mm.

In some embodiments, medicament tablet 1102 has a height of up to 3 mm, up to 4 mm, or up to 5 mm.

In some embodiments, medicament tablet 1102 has a volume of up to 100 mm³, up to 150 mm³, up to 200 mm³, up to 250 mm³, or up to 300 mm³.

In some embodiments, the ingestible medicament of medicament tablet 1102 is absorbable or at least partially absorbable in the stomach of the user. In some embodiments, the ingestible medicament of medicament tablet 1102 is absorbable or at least partially absorbable in the small intestine of the user.

In some embodiments, the ingestible medicament of medicament tablet 1102 is suitable for treatment of one or more symptom or disease, selected from the group consisting of:
Parkinsonism
Parkinson's Disease
progressive supranuclear palsy
corticobasal degeneration
multiple system atrophy
Parkinson-plus syndromes (also known as disorders of multiple system degeneration)
any neurodegenerative disease in which the subject exhibits at least one (and typically at least two or three) of the classical features of Parkinson's disease: tremor, postural instability, and akinesia or bradykesia.
any neurodegenerative disease in which the subject positively responds to a dopaminergic treatment.
any neurodegenerative disease in which the particular subject positively responds to an anticholinergic treatment.
Constipation
Crohn's disease
Gastroparesis
irritable bowel syndrome (IBS)
diarrhea or loose bowel movements
colitis
Hirschsprung's disease
Dyspepsia
dysphagia.

In some embodiments, the ingestible medicament of medicament tablet 1102 comprises or includes an ingestible medicament selected from the group consisting of:
levodopa
at least one dopaminergic agent
at least one catecholamine precursor
a dopamine precursor
at least one dopamine precursor agent
(L)-3,4-dihydroxyphenylalanine
N-methyl-N-(2-propynyl)-2-methyl-1-phenylethyl-2-amine
tyrosine hydroxylase
apomorphine
at least one anticholinergic agent
at least one agent selected to antagonize at least one cholinergic receptor
benzhexol
orphenadrine
at least one selective allosteric potentiator of metabotropic glutamate receptor 4 (mGluR4)
N-phenyl-7-(hydroxylimino)cyclopropa[b]chromen-1a-carboxamide
at least one osmotic agent
magnesium citrate
magnesium hydroxide
polyethylene glycol
sodium phosphate
MiraLAX®
at least one contraction stimulating agent
bisacodyl
senna
Correctol
Ducodyl
Dulcolax
Senexon
Senokot
at least one stool softening agent
docusate sodium
Colace
Linaclotide
Lactulose
lubipro stone
plecanatide
prucaltride
loperamide
bismuth subsalicylate

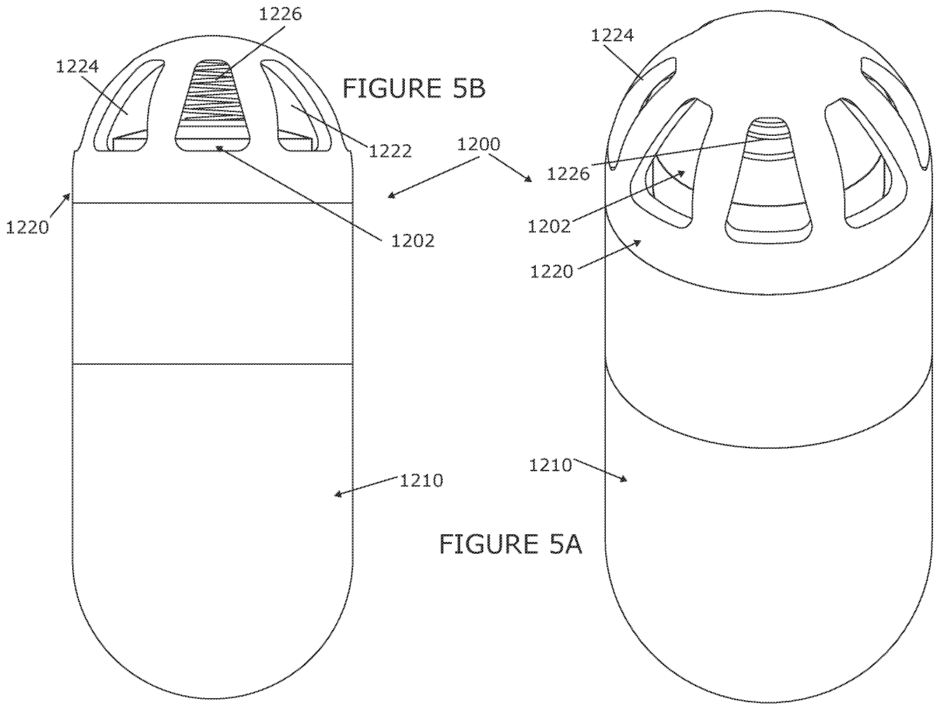

Reference is now made to FIGS. 5A and 5B which are, respectively, a perspective view illustration and a planar side view illustration of a first embodiment of a device 1200 for delivering an ingestible medicament into the gastrointestinal tract of a user, the device having a medicament tablet 1202 including the ingestible medicament disposed therein, according to an embodiment of the present invention, to FIGS. 6A and 6B which are, respectively, a complete and a partial perspective sectional illustration of device 1200 and medicament tablet 1202 disposed therein, and to FIGS. 7A and 7B which are, respectively, a complete and a partial planar sectional illustration of device 1200 having medicament tablet 1202.

As seen, device 1200, which is arranged along a longitudinal axis 1204, includes a vibrating ingestible capsule 1210 including a housing 1212, substantially as described hereinabove with respect to FIG. 4. It will be appreciated that capsule 1210 includes at least a vibrating agitator, a control element, and a power source, as described hereinabove with respect to FIG. 4, even though these components are not explicitly shown in FIGS. 5A to 7B.

A hollow medicament compartment housing 1220, here illustrated as having the shape of a convex dome, defines a hollow 1222 in which is disposed medicament tablet 1202. A plurality of apertures 1224 are formed about the perimeter of medicament compartment housing 1220. In the illustrated embodiment, the apertures 1224 are not large enough to allow passage of the whole medicament tablet 1202 therethrough, although in other embodiments the apertures may be sized and configured to allow a user to insert the medicament tablet into hollow 1222 via one of the apertures 1224.

Hollow medicament compartment housing 1220 is attached to vibrating ingestible capsule 1210. In the illustrated embodiment, housing 1212 of vibrating ingestible capsule 1210 includes a first attachment mechanism in the form of a circumferential slot 1214 and a circumferential protrusion 1216 disposed adjacent a longitudinal end 1218 of capsule housing 1212. Hollow medicament compartment housing includes a second, corresponding attachment mechanism in the form of a circumferential slot 1228 and a circumferential protrusion 1230 disposed adjacent an end 1232 of medicament compartment housing 1220. Circumferential slot 1228 corresponds in dimensions to circumferential protrusion 1216 of capsule 1210, and circumferential protrusion 1230 corresponds in dimensions to circumferential slot 1214 of capsule 1210.

In the illustrated embodiment, medicament compartment housing 1220 is fixedly attached to vibrating ingestible capsule 1210 by snap fit engagement of slot 1228 with protrusion 1216 and snap fit engagement of protrusion 1230 with slot 1214. However, any type of attachment between medicament compartment housing 1220 and vibrating ingestible capsule 1210 is considered within the scope of the present invention, including threaded engagement, engagement by soldering, engagement by adhesive, and the like. For example, in some embodiments, the attachment mechanism of the capsule may included an external thread, adapted to attach to an internal thread on the hollow medicament compartment. As another example, in some embodiments, the attachment mechanism may include an adhesive or an epoxy applied between the vibrating ingestible capsule and the hollow medicament compartment. As a further example, in some embodiment, the attachment mechanism includes at least one fastener fastening the capsule to the hollow medicament compartment.

As seen clearly in FIGS. 6A to 7B, an exterior surface of longitudinal end 1218 of capsule housing 1212 is concave, and has an exterior contour which matches an exterior contour of medicament tablet 1202. However, it will be appreciated that this structure is not necessary, and that medicament tablet 1202 may engage any portion of the capsule housing 1212, along a surface of the capsule housing, along a surface of the medicament tablet, or tangentially.

A biasing mechanism in the form of a compression spring 1226, terminating in a compression plate 1227, extends from the interior surface of hollow medicament compartment housing 1220 toward medicament tablet 1202. Compression spring 1226 and compression plate 1227 bias medicament tablet 1202 toward an exterior surface of housing 1212 of vibrating ingestible capsule 1210. In the illustrated embodiment, the compression spring and compression plate extend from an apex of the dome of medicament compartment housing 1220, and apply pressure to medicament tablet 1202 in a longitudinal direction, along a longitudinal axis of the medicament tablet and of device 1200. However, any other suitable arrangement of the biasing mechanism is considered to be within the scope of the invention, as described hereinbelow with respect to FIGS. 9A and 9B.

Reference is now additionally made to FIGS. 8A and 8B, which are, respectively, partial sectional illustrations of device 1200, at two times during use thereof. As seen, in FIG. 8A, the medicament tablet 1202 is larger, or is whole, and is compressed by compression spring 1226 and compression plate 1227 against end 1218 of housing 1212 of the vibrating ingestible capsule 1210. At a later time, occurring for example after the device 1200 has been ingested and the medicament tablet 1202 has been exposed to gastric fluids, the medicament tablet is significantly smaller than it was at the starting time. However, the compression spring 1226 and compression plate 1227 continue to bias the medicament tablet against the end 1218 of capsule housing 1212.

As such, it is appreciated that the longitudinal biasing mechanism illustrated in FIGS. 8A and 8B of the present invention “follows” the medicament tablet, and continues to bias the medicament tablet in a desired direction during changes to the dimensions of the medicament tablet, and during absorption of the medicament therefrom.

Reference is now additionally made to FIGS. 9A and 9B, which are, respectively, partial sectional illustrations of a device similar to device 1200 and having a radial biasing mechanism, at two times during use thereof, according to another embodiment of the present invention. Specifically, sectional illustrations 9A and 9B are taken in a direction perpendicular to the longitudinal axis of the device, through the center of the medicament tablet.

As seen in FIGS. 9A and 9B, a hollow medicament compartment housing 1250 has a medicament tablet 1252 disposed therein. A biasing mechanism, including a plurality of flexible and resilient leaves 1254 extending radially inwardly from an interior surface of hollow medicament compartment housing 1250 applies pressure, in a radial direction, to a perimeter of medicament tablet 1252, so as to bias the medicament tablet toward the center of medicament compartment housing 1250 and to hold it in place there.

As seen, in FIG. 9A, the medicament tablet 1252 is larger, or is whole, and is held by biasing leaves 1254 to be concentric with a hollow medicament compartment housing 1250. At a later time, occurring for example after the device has been ingested and the medicament tablet 1252 has been exposed to gastric fluids, the medicament tablet is significantly smaller than it was at the starting time. However, the biasing leaves 1254 continue to bias the medicament tablet 1252 to be concentric with a hollow medicament compartment housing 1250.

As such, it is appreciated that the radial biasing mechanism illustrated in FIGS. 9A and 9B of the present invention “follows” the medicament tablet, and continues to bias the medicament tablet in a desired direction during changes to the dimensions of the medicament tablet, and during absorption of the ingestible medicament therefrom.

Reference is now made to FIGS. 10A and 10B, which are, respectively, a perspective view illustration and a planar view illustration of a second embodiment of a device 1300 for delivering an ingestible medicament into the gastrointestinal tract of a user, the device having a medicament tablet 1302 including the ingestible medicament disposed therein, according to yet another embodiment of the present invention, and to FIGS. 11A and 11B, which are, respectively, a complete and a partial planar sectional illustration of device 1300 having medicament tablet 1302 disposed therein.

As seen, device 1300, which is arranged along a longitudinal axis 1304, includes a vibrating ingestible capsule 1310 including a housing 1312, substantially as described hereinabove with respect to FIG. 4. It will be appreciated that capsule 1310 includes at least a vibrating agitator, a control element, and a power source, as described hereinabove with respect to FIG. 4, even though these components are not explicitly shown in FIGS. 10A to 11B.

A hollow medicament compartment housing 1320, here illustrated as having the shape of a convex domed sleeve, defines a hollow 1322 in which is disposed medicament tablet 1302. A plurality of circular apertures 1324 are formed about the perimeter of medicament compartment housing 1320. In the illustrated embodiment, the apertures 1324 are not large enough to allow passage of the whole medicament tablet 1302 therethrough, although in other embodiments the apertures may be sized and configured to allow a user to insert the medicament tablet into hollow 1322 via one of the apertures 1324.

Hollow medicament compartment housing 1320 partially envelops vibrating ingestible capsule 1310. In some embodiments, the medicament compartment housing may frictionally engage housing 1312 of capsule 1310.

In other embodiments, not illustrated, hollow medicament compartment housing 1320 may fully envelop ingestible vibrating capsule 1310. In some such embodiments, hollow medicament compartment housing 1320 may form a hollow capsule, having a greater diameter than capsule 1310 and a greater length than capsule 1310, such that capsule 1310 as well as medicament tablet 1302 may be disposed, adjacent one another, within the capsule of medicament compartment housing 1320.

As seen in FIGS. 11A and 11B, medicament tablet 1302 is disposed within and engages housing 1312 of vibrating ingestible capsule 1310, without being actively biased toward the capsule 1310. As such, when capsule 1310 is in its operative vibration mode, vibration of the capsule 1310 affects the tablet 1302 as well as the medicament compartment housing 1320 surrounding the capsule 1310, and promotes absorption of the ingestible medicament from tablet 1302 substantially as described hereinabove.

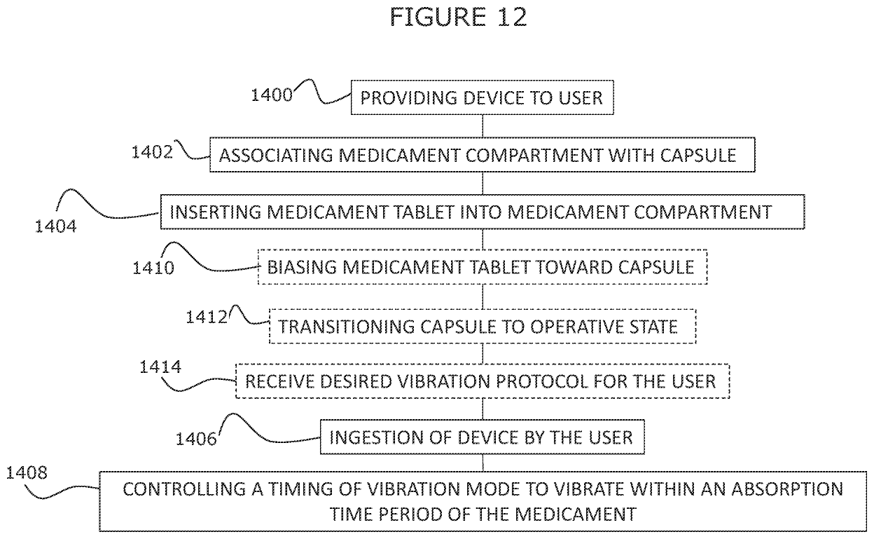

Reference is now additionally made to FIG. 12, which is a schematic flowchart of a method for delivering an ingestible medicament of a medicament tablet into the gastrointestinal tract of user according to the present invention. The method may be based on the use of a device including a vibrating ingestible capsule and a medicament tablet, as described hereinabove with reference to FIGS. 4 to 11B.

As seen at step 1400, a device, such as device 1100, 1200, or 1300 described hereinabove, including a vibrating ingestible capsule and a hollow medicament compartment housing, is provided to a user.

At step 1402, the vibrating ingestible capsule and the hollow medicament compartment housing are associated with one another. In some embodiments, step 1402 may take place in a factory, prior to providing the device to the user at step 1400. In other embodiments, the device may be provided to the user as two separate pieces, namely the ingestible vibrating capsule and the hollow medicament compartment housing, and the user carries out step 1402 following receipt of the device at step 1400.

In some embodiments, the associating at step 1402 includes at least partially attaching the medicament compartment housing to the vibrating ingestible capsule.

In some embodiments, the associating at step 1402 includes fully attaching the medicament compartment housing to the vibrating ingestible capsule.

In some embodiments, the associating at step 1402 includes fixedly attaching the medicament compartment housing to the vibrating ingestible capsule.

In some embodiments, the associating at step 1402 includes removably attaching the medicament compartment housing to the vibrating ingestible capsule.

In some embodiments, the associating at step 1402 includes attaching the medicament compartment housing to the vibrating ingestible capsule by one or more of snap fit engagement, threaded engagement, adhering, soldering, or any other suitable mechanism of attachment.

In some embodiments, the associating at step 1402 includes mutually attaching a first attachment mechanism on the vibrating ingestible capsule with a corresponding attachment mechanism on the medicament compartment housing, for example as described with respect to FIGS. 5A to 8B.

In some embodiments, the associating at step 1402 includes at least partially enveloping the medicament compartment housing around the vibrating ingestible capsule.

In some embodiments, the associating at step 1402 includes fully enveloping the medicament compartment housing around the vibrating ingestible capsule.

In some embodiments, the device is provided to the user having the medicament tablet disposed within the hollow medicament compartment housing.

At step 1404, the medicament tablet is inserted into the hollow medicament compartment housing. In some embodiments, step 1404 may take place prior to associating the ingestible vibrating capsule with the hollow medicament compartment housing, either in a factory or by the user. In other embodiments, step 1404 may be carried out following association of the ingestible vibrating capsule with the hollow medicament compartment housing, for example by insertion of the medicament tablet into the hollow via one of the apertures in the hollow medicament compartment housing. The medicament tablet inserted at step 1404 may be any suitable type of medicament tablet, as described in detail hereinabove.

At step 1406, the device, including the vibrating ingestible capsule, the hollow medicament compartment housing, and the medicament tablet, is ingested by the user, and begins to travel through the gastrointestinal tract of the user.

At step 1408, which occurs following the user ingesting the device at step 1406, the vibrating ingestible capsule is controlled such that the vibration mode of operation (e.g., when the vibration mode is initiated, a duration of the vibration mode, etc.) at least partially transpire within an absorption time period of the ingestible medicament included in the medicament tablet within the gastrointestinal tract of the user.

The absorption time period may be an estimated absorption time period, as defined herein, and/or an actual absorption time period as defined herein.

In some embodiment, step 1408 may include controlling a timing of the vibration mode of operation such that the vibration mode at least partially transpires when the capsule is in a region of the gastrointestinal tract in which the ingestible medicament is typically absorbed into the bloodstream. The region of the gastrointestinal tract may include one or more of the stomach of the user, the duodenum of the user, the small intestine of the user, the large intestine of the user, or the colon of the user.

For example, when the medicament tablet includes the ingestible medicament levadopa, which is typically absorbed into the bloodstream through the stomach walls and/or the small intestine walls, the vibration mode at least partially transpires within a time period in which the device traverses, or is expected to traverse, the stomach and small intestine.

In some embodiments, step 1408 includes setting at least one vibration parameter of the vibrating ingestible capsule of the device so as to promote absorption of the ingestible medicament into the bloodstream of the user. In some such embodiments, the at least one vibration parameter set at step 1408 includes at least one of a vibration frequency, a cumulative vibration duration, a number of vibration cycles per time unit, a duration of a vibration duration within a vibration cycle, a duration of a repose duration within a vibration cycle, a total duration of a single vibration cycle, and a net force exerted by said housing on said environment.

In some embodiments, the controlling at step 1408 includes controlling the vibrating agitator such that the vibrating mode of operation includes a plurality of cycles, each of the cycles including a vibration duration followed by a repose duration, wherein the housing exerts the vibrations during the vibration duration.

In some embodiments, the repose duration is greater than the vibration duration.

In some embodiments, the vibration duration is in the range of 0.1 second to 10 seconds, 1 second to 10 seconds, 1 second to 9 seconds, 2 seconds to 9 seconds, 3 seconds to 9 seconds, 3 seconds to 8 seconds, 3 seconds to 7 seconds, 3 seconds to 6 seconds, 4 seconds to 6 seconds, or 5 seconds to 6 seconds.

In some embodiments, the repose duration is in the range of 1 second to 180 seconds, 3 seconds to 180 seconds, 5 seconds to 180 seconds, 5 seconds to 150 seconds, seconds to 120 seconds, 8 seconds to 100 seconds, 8 seconds to 30 seconds, 10 seconds to 80 seconds, 10 seconds to 70 seconds, 10 seconds to 60 seconds, 10 seconds to 50 seconds, 10 seconds to 40 seconds, 10 seconds to 30 seconds, 10 seconds to 20 seconds, or 15 seconds to 20 seconds.

In some embodiments, a duration of each of the plurality of cycles is in the range of 1.1 seconds to 200 seconds, 5 seconds to 200 seconds, 10 seconds to 200 seconds, 10 seconds to 150 seconds, 10 seconds to 100 seconds, 10 seconds to 80 seconds, 10 seconds to 50 seconds, 10 seconds to 40 seconds, 10 seconds to 30 seconds, 15 seconds to 50 seconds, 15 seconds to 40 seconds, 15 seconds to 30 seconds, or 15 seconds to 25 seconds.

In some embodiments, the controlling at step 1408 includes controlling the vibrating agitator such that a cumulative duration of the vibrating mode of operation is in the range of 1 hour to 12 hours, 2 hours to 10 hours, 2 hours to 8 hours, 2 hours to 6 hours, 2 hours to 4 hours, or 2 hours to 3 hours.

In some embodiments, the controlling at step 1408 includes controlling the vibrating agitator to exert forces on the housing of the vibrating ingestible capsule, such that a net force exerted by the housing on the environment thereof is in the range of 50 grams force (gf) to 600 gf, 50 gf to 550 gf, 100 gf to 550 gf, 100 gf to 500 gf, 150 gf to 500 gf, 200 gf to 500 gf, or 200 gf to 450 gf.

In some embodiments, the controlling at step 1408 includes controlling the vibrating agitator to exert the forces on the housing to attain a housing vibrational frequency within a range of 10 Hz to 650 Hz, 15 Hz to 600 Hz, 20 Hz to 550 Hz, 30 Hz to 550 Hz, 50 Hz to 500 Hz, 70 Hz to 500 Hz, 100 Hz to 500 Hz, 130 Hz to 500 Hz, or 150 Hz to 500 Hz.

In some embodiments, the method may also include step 1410, in which the medicament tablet is biased toward the vibrating ingestible capsule. Step 1410 may a continuous step, which begins when the medicament tablet is inserted into the hollow medicament compartment housing at step 1404, and continues as long as the medicament tablet has not been removed from the hollow medicament compartment housing and has not been fully dissolved. More specifically, step 1410 may continuously occur during ingestion of the device by the user at step 1406, and in some embodiments during at least part of the duration controlling of the vibrating agitator at step 1408.

In some embodiments, the biasing at step 1410 includes biasing the medicament tablet by application of pressure along a longitudinal axis of the medicament tablet.

In some embodiments, the biasing at step 1410 includes biasing the medicament tablet by application of radial pressure along a perimeter of the medicament tablet.

In some embodiments, and as described in further detail herein, the method may include a further step 1412 of transitioning the capsule (from an inoperative state) to an operative state.

The capsule may be pre-programmed with a vibration protocol. This protocol may include, by way of example, a particular or pre-determined activation time following ingestion, in which the capsule is transitioned from an inoperative state to an operative state. In such embodiments, the step 1412 may be omitted from the method.

Alternatively or additionally, the capsule may receive an activation input in an active fashion (e.g., from an external controller via RF) or in a passive fashion (e.g., a signal from a sensor to the on-board controller), as described in detail hereinabove. It will be appreciated that step 1412, in which the vibrating ingestible capsule is transitioned from the inoperative state to the operative state, may be performed prior to ingestion of the device by the user in step 1406, or following such ingestion, for example in the case of external control via RF.

Substantially as described hereinabove, step 1412 may be carried out, and the vibrating ingestible capsule may be activated, prior to the user ingesting the capsule at step 1406, for example by a signal from the control unit or by the user carrying out an activation motion. In other embodiments, the activation input, and the transitioning of the capsule from being inoperative to being operative, occurs at the time of ingestion or immediately thereafter, for example by sensors sensing a change in the environment of the capsule due to its ingestion, as described at length hereinabove. In yet other embodiments, the transitioning of the capsule at step 1412 may include the capsule receiving an activation input which is provided remotely when the capsule is already in the body of the user, for example by remote communication from control module 1140.

In some embodiments, a control element of the vibrating ingestible capsule may optionally receive a desired vibration protocol for the user, at an optional step 1414. In some embodiments, the programming of the desired vibration protocol at step 1414 occurs at the time of manufacturing of the vibrating ingestible capsule or of the device, for example by pre-programming the protocol into the control element. In other embodiments, providing the desired vibration protocol for the user at step 1414 may be effected by a control unit, such as control unit 1140 of FIG. 4, as described in detail hereinabove with respect to FIG. 4.

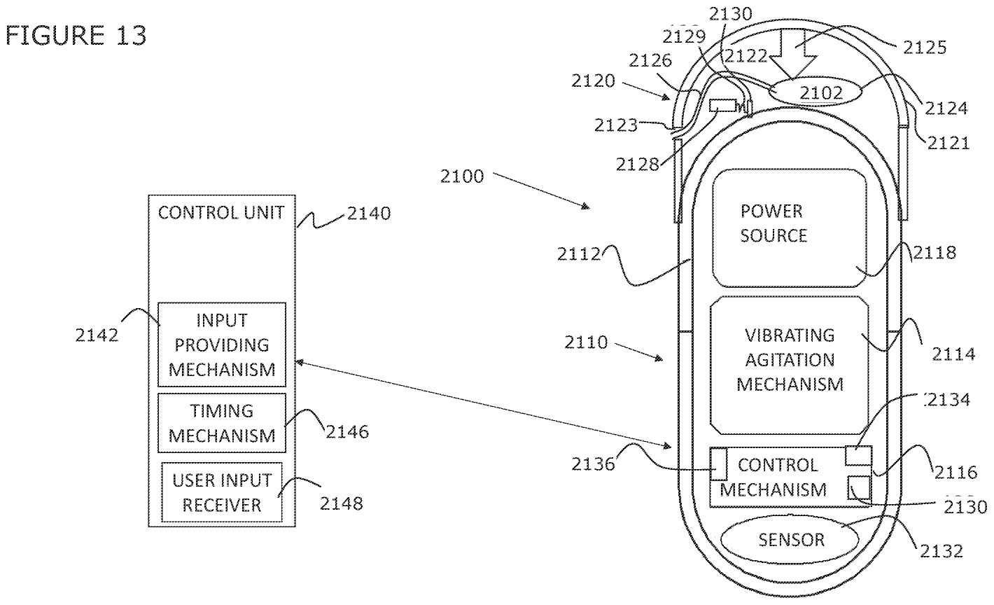

Reference is now made to FIG. 13, which is a schematic block diagram of a device 2100 for delivering a flowable ingestible medicament 2102 into the gastrointestinal tract of a user according to an embodiment of the present invention.

It is a particular feature of the present invention that delivery of the flowable ingestible medicament is triggered by, and occurs only during a time at which a vibrating ingestible capsule is a vibration mode of operation, as explained in detail hereinbelow. Stated differently, delivery of the flowable ingestible medicament only occurs when the vibrating ingestible capsule is actually vibrating, and is triggered by such vibration of the vibrating ingestible capsule.

As seen in FIG. 13, device 2100 includes vibrating ingestible capsule 2110. Capsule 2110 includes a capsule housing or shell 2112, also termed a first housing portion, arranged along a longitudinal axis 2113 and having disposed therein a vibrating agitator 2114. A control element 2116 is adapted to control operation of vibrating agitator 2114, and at least one power source 2118 provides power to vibrating agitator 2114 and control element 2116.

The vibrating ingestible capsule 2110 is functionally associated with a medicament delivery compartment 2120.

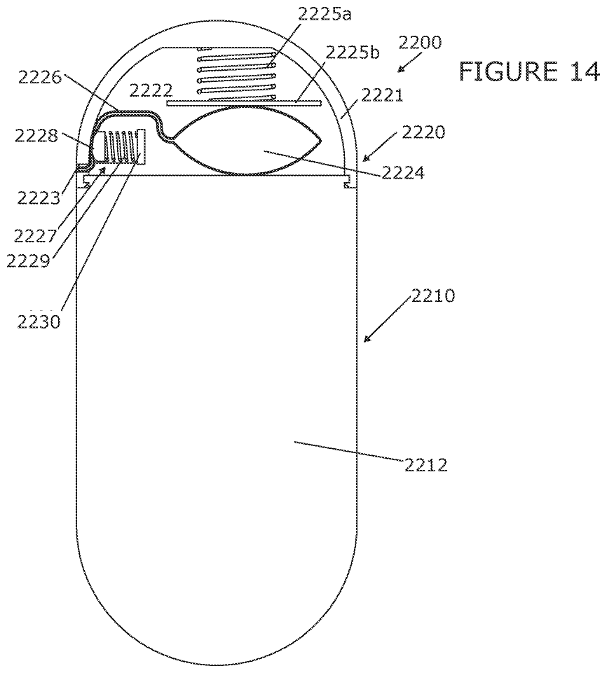

In some embodiments, the medicament delivery compartment 2120 is distinct from the vibrating ingestible capsule 2110, and is attached thereto, as illustrated in FIG. 13 and as explained hereinbelow with respect to FIG. 14. In such embodiments, the medicament delivery compartment includes a hollow medicament compartment housing 2121, also termed a second housing portion, defining a hollow 2122. The hollow compartment housing includes a portal 2123 and is attached to housing 2112 of capsule 2110.

Medicament compartment housing 2120 may be fixedly attached to housing 2112 of vibrating ingestible capsule 2110, or removable attached thereto. Medicament compartment housing 2120 may be attached to housing 2112 of vibrating ingestible capsule 2110 using any suitable attachment method, such as by adhering, by soldering, by snap fit engagement or by threaded engagement.

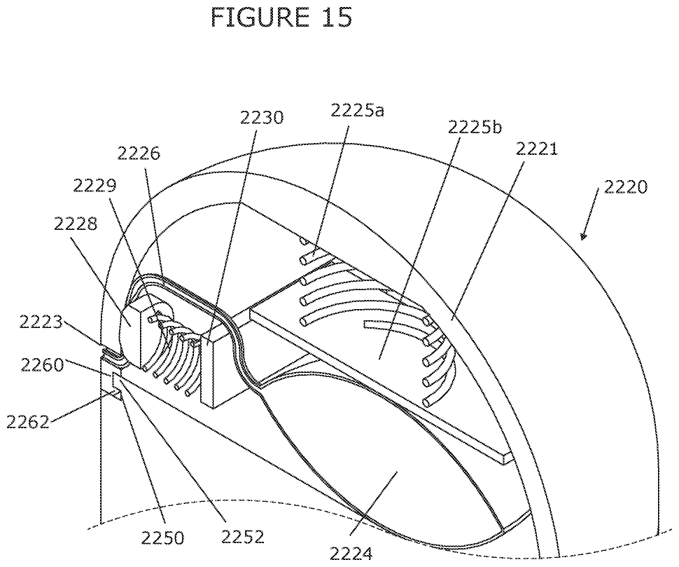

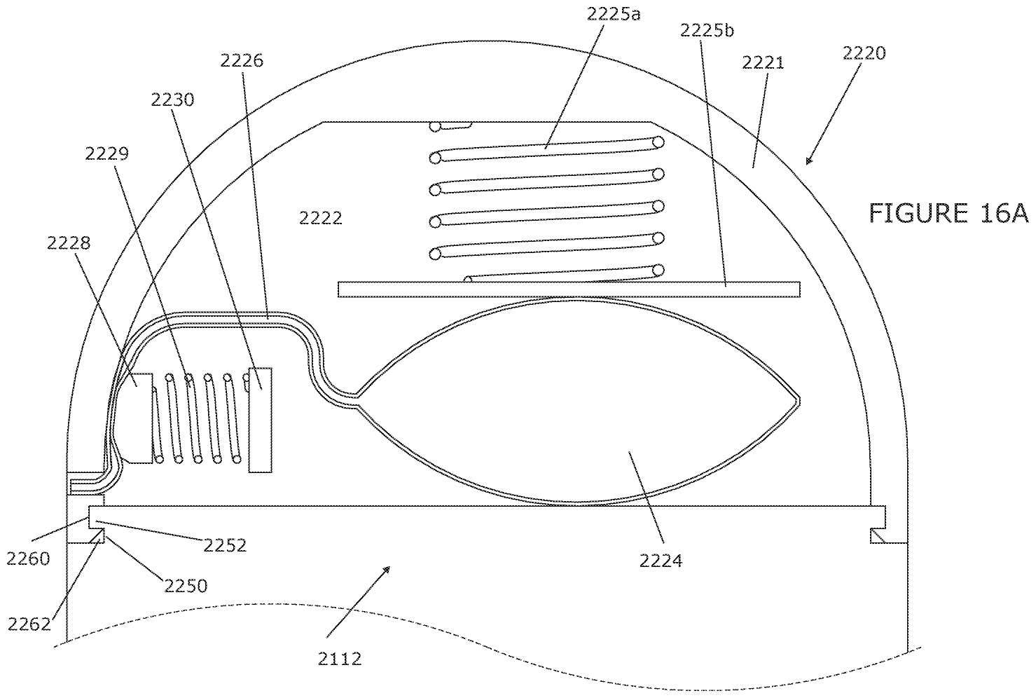

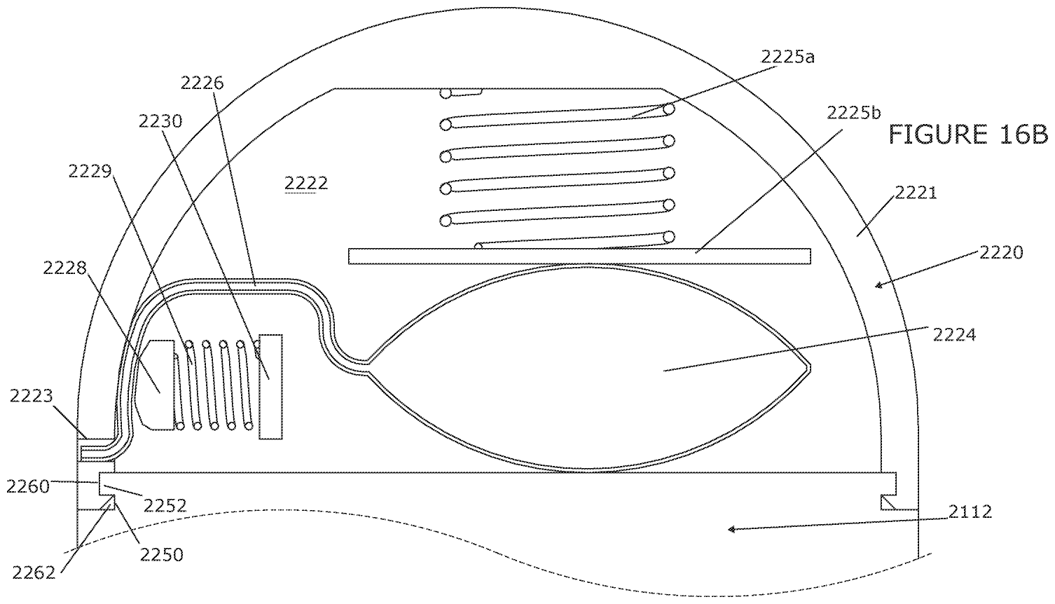

In some embodiments, housing 2112 of vibrating ingestible capsule 2110 includes an attachment mechanism, and hollow medicament compartment housing 2121 includes a corresponding attachment mechanism, for mutual attachment of vibrating ingestible capsule 2110 to hollow medicament compartment housing 2121. An exemplary arrangement of such attachment mechanisms is illustrated, for example, in FIGS. 15 to 16B described in detail hereinbelow.

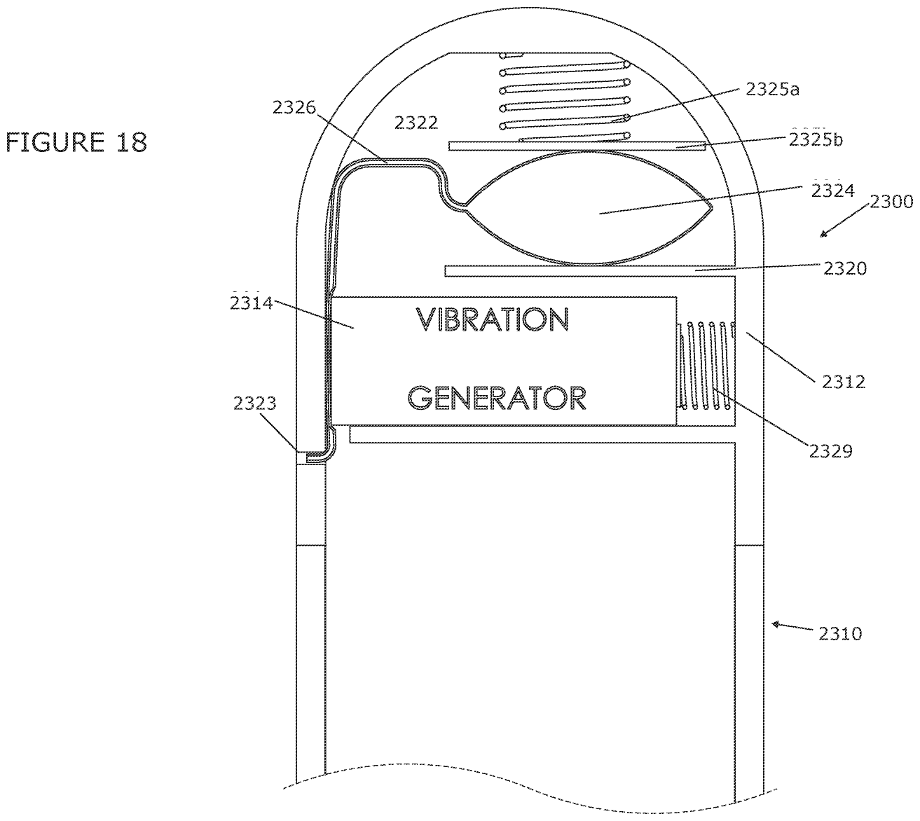

In other embodiments, the medicament delivery compartment 2120 may form part of the vibrating ingestible capsule 2110, as explained hereinbelow with respect to FIG. 18. In such embodiments, the portal 2123 is formed in housing 2112, and the hollow of the vibrating ingestible capsule includes all components otherwise included in hollow 2122, as explained hereinbelow.

A flexible and collapsible medicament reservoir 2124, which is adapted to have the flowable ingestible medicament 2102 disposed therein, is disposed within hollow 2122, and pressure is constantly applied thereto by a reservoir biasing mechanism 2125, which may be anchored to medicament compartment housing 2121. A conduit 2126, which may be a flexible and/or resilient conduit, extends from medicament reservoir 2124 to portal 2123, such that a fluid flowing through the conduit is delivered to an environment surrounding device 2100. Typically, conduit 2126 seals portal 2123, to prevent fluid from the environment surrounding device 2100 from entering the device.

A valve, which includes a weight 2128 and a valve biasing mechanism 2129, typically a spring, adapted, in a closed operative orientation, to block flow through the conduit 2126 by biasing weight 2128 against the conduit thereby to pinch the conduit, and, in an open operative orientation, to remove weight 2128 from applying pressure to conduit 2126, such that, following the recovery time of the conduit, fluid may flow through the conduit.

It is a particular feature of the present invention that valve biasing mechanism 2129 is functionally associated with housing 2112 or with vibrating agitator 2114. When vibrating agitator 2114 is in an inoperative state, or is operative but not in a vibration mode of operation, valve biasing mechanism 2129 biases weight 2128 against conduit 2126, such that the valve is in the closed operative orientation and fluid cannot flow through the conduit. When vibrating agitator 2114 is in a vibration mode of operation and exerts vibrations on housing 2112, at least some of the exerted vibrations are applied to valve biasing mechanism 2129, and cause the valve biasing mechanism to move periodically. Periodic motion of the valve biasing mechanism 2129 results in corresponding periodic motion of weight 2128 away from conduit 2126, thereby transitioning the valve from the closed operative orientation to an open operative orientation and allowing fluid to flow through conduit 2126.

Due to the flexibility and resiliency of conduit 2126, when the weight 2128 is moved away from the conduit, conduit 2126 at least partially recovers such that fluid can flow through the conduit. Because reservoir biasing mechanism 2125 constantly applies pressure to reservoir 2124, upon recovery of conduit 2126, enables the flowable ingestible medicament flows through the conduit, and out of portal 2123, into an environment surrounding the device 2100. As such, delivery of the flowable ingestible medicament is triggered by, and controlled by, vibration of the vibrating agitator 2114.

The rate at which the flowable ingestible medicament is delivered into the environment surrounding device 2100 is dependent on characteristics of the conduit 2126, such as the diameter of the conduit, the thickness of the conduit walls, and the recovery time of the conduit, as well as on characteristics of the valve, such as the frequency of transitioning between the open and closed operative orientations of the valve.

It will be appreciated that the frequency at which valve biasing mechanism 2129 transitions the valve between the open and closed operative orientations, by causing weight 2128 to move towards and away from conduit 2126, is dependent on the frequency of vibrations exerted by vibrating agitator 2114 or by housing 2112, as well as on the characteristics of the valve, such as the mass of weight 2128 and the spring constant of the biasing mechanism 2129.

In some embodiments, the mass of weight 2128 and the length and spring constant of biasing mechanism 2129 may be selected such that the valve functions as a gear reducer. In such embodiments, the frequency at which the valve transitions between the closed and open operative orientations (i.e. the frequency at which the valve biasing mechanism 2129 draws weight 2128 away from conduit 2126 and then pushes the weight 2128 back to apply pressure to conduit 2126) may be smaller than the frequency of vibrations exerted by vibrating agitator 2114.

In some embodiments, valve biasing mechanism 2129 is anchored to housing 2112. In some embodiments, valve biasing mechanism 2129 may be anchored to medicament compartment housing 2121, provided that the attachment between the medicament compartment housing 2121 and housing 2112 of vibrating ingestible capsule 2110 does not dampen or reduce the frequency or intensity of vibrations applied to valve biasing mechanism 2129, and that the frequency and intensity of vibrations exerted on medicament compartment housing 2121 is substantially equal to that of vibrations exerted on housing 2112.

Relating now to characteristics of the medicament delivery compartment 2120, in some embodiments, in which the medicament delivery compartment 2120 is distinct from vibrating ingestible capsule 2110, hollow 2122 of medicament compartment housing 2121 has a volume in the range of 200 mm³ to 800 mm³, 300 mm³ to 700 mm³, or 400 mm³ to 600 mm³. In other embodiments, in which housing 2112 houses components of medicament delivery compartment 2120, the housing 2112 has a volume in the range of 1000 mm³ to 4000 mm³.

In some embodiments, medicament reservoir 2124 has a maximal volume in the range of 1 mm³ to 600 mm³, 1 mm³ to 10 mm³, 5 mm³ to 20 mm³, 15 mm³ to 50 mm³, 30 mm³ to 200 mm³, 100 mm³ to 400 mm³, or 300 mm³ to 600 mm³.

In some embodiments, medicament reservoir 2124 has elastic or elastomeric properties, and may have a low value for Young's modulus, typically smaller than 1 GPa. In some embodiments, medicament reservoir 2124 is formed of a material selected from the group consisting of: silicone rubber, natural rubber, Polyethylene, and PVC.

In some embodiments, reservoir biasing mechanism 2125 includes a reservoir spring terminating in a pressure applying surface which engages an exterior surface of medicament reservoir 2124, as illustrated in FIGS. 14 to 17. In such embodiments, the reservoir spring may be anchored to medicament compartment housing 2121.

In some embodiments, reservoir biasing mechanism 2125 has a spring constant K in the range of 1 N/m to 200 N/m.

In some embodiments, flexible and resilient conduit 2126 is integrally formed with flexible and collapsible medicament reservoir 2124. In other embodiments, conduit 2126 is formed of a different material than medicament reservoir 2124.

In order to ensure that when vibrating agitator 2114 is in the vibration mode of operation, fluid can be delivered through conduit 2126, the recovery time of the conduit must be sufficiently short for the conduit to recover its nominal diameter, and facilitate passage of fluid therethrough, before the conduit is once again pinched by weight 2128.

As such, in some embodiments, when vibrating agitator has a frequency f, a recovery time of conduit 2126 is at most equal to 1/f. In other embodiments, when valve biasing mechanism 2129 has a frequency fv of transitioning the valve between the open and closed operative orientations, a recovery time of conduit 2126 is at most equal to 1/fv.

In some embodiments, a recovery time of conduit 2126 is at most 0.1 seconds.

In some embodiments, conduit 2126 has a diameter in the range of 0.01 mm-0.9 mm.

In some embodiments, conduit 2126 has a length in the range of 3 mm-25 mm. In some embodiments, valve biasing mechanism 2129 has a spring constant in the range of 0.1 N/m to 5 N/m.

In some embodiments, weight 2128 has a mass in the range of 0.1 grams to 2 grams.

Relating specifically to capsule 2110, power source 2118 may be any suitable power source, such as one or more alkaline or silver oxide batteries, primary batteries, rechargeable batteries, capacitors and/or supercapacitors.

Intermittently activated vibrating agitator 2114 is adapted to have a vibration mode of operation (also termed the first mode of operation) and a rest mode of operation (also termed the second mode of operation). In the vibration mode of operation, intermittently activated vibrating agitator 2114 is adapted to exert forces on capsule housing 2112, such that capsule housing 2112 exerts vibrations on an environment surrounding capsule 2110 and/or device 2100.

In some embodiments, the capsule 2110 is in an inoperative state, until the receipt of an activation input, which causes control element 2116 to transition the capsule from the inoperative state to an operative state.

In some embodiments, control element 2116 is functionally associated with, or includes, a timer or timing mechanism 2130, such as a clock, universal clock, or stopwatch, powered by power source 2118 and adapted to track at least one time characteristic, such as a duration that has passed since an activation input was received, or a duration that has passed since the user ingested capsule 2110.

In some embodiments, capsule 2110 is devoid of any sensors for sensing an environment thereof. In some such embodiments, control element 2116 is adapted, in response to receipt of an activation input, to wait a predetermined delay time, and following the predetermined delay time, to activate vibrating agitator 2114 to operate in said first vibration mode of operation.

In other embodiments, such as the embodiment illustrated in FIG. 13, capsule 2110 further includes at least one sensor 2132, functionally associated with control element 2116. The at least one sensor 2132 may be adapted to sense at least one parameter within capsule 2110 or in an environment of capsule 2110, and may include a temperature sensor, an illumination sensor, a moisture sensor, a pressure sensor, an accelerometer, or any other suitable sensor. In some embodiments, the at least one sensor 2132 is adapted to identify a specific condition in capsule 2110 or in the vicinity thereof, and to provide an activation input to control element 2116 in response to identification of the condition. For example, in some embodiments the condition is indicative of the user ingesting capsule 2110.

For example, in some embodiments sensor 2132 may include an illumination sensor, adapted to identify transition of capsule 2110 from an illuminated environment (e.g. outside the human body) to a dark environment (e.g. within the human body) and to provide an activation input in response to identification of such a transition.

As another example, in some embodiments sensor 2132 may include a motion or acceleration sensor, such as an accelerometer, adapted to identify an activation motion carried out by a user on capsule 2110 or on device 2100 and to provide an activation input in response to identification of such a transition. An example of an accelerometer providing an activation input for a gastrointestinal capsule is provided in U.S. Pat. No. 10,314,514, which is incorporated by reference for all purposes as if fully set forth herein.

As another example, in some embodiments sensor 2132 may include a pressure sensor adapted identify pressure applied to the capsule 2110 or to device 2100, which pressure is indicative of the capsule moving through a pharynx of the user, and to provide an activation input in response to identification of such pressure.

As a further example, in some embodiments sensor 2132 may include a temperature sensor adapted to identify transition of capsule 2110 or of device 2100 from an area with ambient temperature (e.g. outside the human body) to an area with a human body temperature and to provide an activation input in response to identification of such a transition.

As a further example, in some embodiments sensor 2132 may include a moisture sensor adapted to identify transition of capsule 2110 or of device 2100 from a dry area (e.g. outside the human body) to a moist area (e.g. within the human body) and to provide an activation input in response to identification of such a transition.

It will be appreciated by people of skill in the art that sensor 2132 need not necessarily be disposed within capsule 2110, as illustrated in FIG. 13, and may be disposed anywhere within device 2100, for example within hollow 2122 of medicament compartment housing 2120, on an exterior of capsule 2110, or on the exterior of device 2100.

In some embodiments, device 2100 may be functionally associated with a control unit 2140, which may be remote from device 2100 and from capsule 2110, and which is adapted to provide one or more inputs to the capsule. In some such embodiments, capsule 2110 further includes a remote input receiving mechanism 2136, functionally associated with control element 2116, and adapted to receive inputs from an input providing mechanism 2142 of control unit 2140.

In some embodiments, control unit 2140 may further include a timing mechanism 2146, adapted to track at least one time characteristic, such as a duration that has passed since a control instruction was provided to capsule 2110.

In some embodiments, control unit 2140 may further include a user input receiver 2148, such as a keyboard, touch screen, or touch pad, adapted to receive input from a user, such as the user, a medical professional treating the user, or a caregiver of the user.

Control unit 2140 may be any suitable type of control unit. In some embodiments, control unit may be a suitably configured smart phone or a tablet computer.

In some such embodiments, control unit 2140 may provide inputs to capsule 2110 by remotely transmitting the inputs from input providing mechanism 2142 to remote input receiving mechanism 2136, for example using a short range wireless communication method, such as radio frequency (RF) communication or Bluetooth® communication. One example of such a mechanism for providing input to a capsule is described in U.S. Pat. No. 10,478,373, which is incorporated by reference for all purposes as if fully set forth herein.

In some embodiments, control unit 2140 is adapted to provide the activation input to control element 2116 of capsule 2110. In some such embodiments, control unit 2140 provides the activation input prior to the user ingesting device 2100 including capsule 2110, whereas in other embodiments control unit 2140 provides the activation input following ingestion of device 2100 and capsule 2110 by the user.

Relating to the characteristics of vibrating agitator 2114, the vibrating agitator may be any suitable mechanism that can be intermittently activated and can apply suitable forces onto capsule housing 2112.

In some embodiments, intermittently activated vibrating agitator 2114 may include a radial agitation mechanism adapted to exert radial forces on capsule housing 2112, in a radial direction with respect to the longitudinal axis of housing 2112. For example, the radial agitation mechanism may include an unbalanced weight attached to a shaft of an electric motor powered by said battery, substantially as described in U.S. Pat. No. 9,707,150, which is incorporated by reference for all purposes as if fully set forth herein.

In some embodiments, intermittently activated vibrating agitator 2114 may include an axial agitation mechanism adapted to exert radial forces on the capsule housing 2112, in an axial direction with respect to a longitudinal axis of housing 2112. For example, the axial agitation mechanism may include an electric motor powered by the battery and an urging mechanism, associated with, and driven by, the electric motor, such that the urging mechanism adapted to exert said axial forces, substantially as described in U.S. Pat. No. 9,707,150. In some embodiments, the urging mechanism adapted to exert the axial forces in opposite directions. In some embodiments, the urging mechanism is adapted to deliver at least a portion of the axial forces in a knocking mode.

In some embodiments, the forces exerted by intermittently activated vibrating agitator 2114 on capsule housing 2112 in the vibration mode of operation include radial forces in a radial direction with respect to the longitudinal axis of the housing and axial forces in an axial direction with respect to the longitudinal axis. In some embodiments, a single agitation mechanism exerts both the radial and the axial forces. In other embodiments, the axial forces are exerted by one agitation mechanism, and the radial forces are exerted by another, separate, agitation mechanism, where both agitation mechanisms form part of intermittently activated vibrating agitator 2114.

In some embodiments, the intermittently activated vibrating agitator 2114 may include a magnet mounted onto a rotor adapted to exert a magnetic field as well as radial forces on capsule housing 2112. For example, such a magnetic vibrating agitator is described in US Patent Application Publication No. 2016/0310357, which is incorporated by reference for all purposes as if fully set forth herein.

In some embodiments, housing 2112 may include first and second members, and vibrating agitator 2114 may include a mechanism adapted to effect a vibration by moving the first member of the housing in the opposite direction relative to the second member of the housing, substantially as described in U.S. Pat. No. 9,078,799, which is incorporated by reference for all purposes as if fully set forth herein.

In some embodiments, housing 2112 may include a vibrating agitator 2114 which makes use of a pendulum to cause vibration in the vicinity of the capsule, for example as described in CN Patent Application Number 105997466 filed on Jun. 16, 2016, which is incorporated by reference for all purposes as if fully set forth herein.

In some embodiments, or at some times, control element 2116 is adapted to control vibrating agitator 2114, and specifically to set at least one vibration parameter of vibrating agitator 2114, so as to promote delivery of the flowable ingestible medicament into an environment surrounding device 2100 and/or absorption of the ingestible medicament into the bloodstream of the user.

For example, delivery of the flowable ingestible medicament into an environment surrounding device 2100 may be promoted by controlling one or more characteristics of the vibrating agitator 2114 such that conduit 2126 is open for relatively long durations, facilitating rapid delivery of the flowable medicament into the gastrointestinal tract.

As another example, absorption of the ingestible medicament may be promoted by the vibration promoting emulsification of the ingestible medicament. As yet another example, absorption of the ingestible medicament may be promoted by the vibration causing a hydrophobic phase of the ingestible medicament to form smaller bubbles, thereby increasing the surface area of the hydrophobic phase for absorption thereof. In another example, absorption of the ingestible medicament may be promoted by the vibration causing greater exposure of the ingestible medicament to the environment.

In some embodiments, or at some times, control element 2116 may be adapted to control vibrating agitator 2114 so that the capsule applies forces to an environment thereof, such that within the gastrointestinal tract, a mechanical stimulation of the wall of the gastrointestinal tract is effected.

In some such embodiments, the at least one vibration parameter includes at least one of a vibration frequency, a cumulative vibration duration, a number of vibration cycles per time unit, a duration of a vibration duration within a vibration cycle, a duration of a repose duration within a vibration cycle, a total duration of a single vibration cycle, and a net force exerted by said housing on said environment, as explained in further detail hereinbelow.

In some embodiments, control element 2116 is adapted to control a timing or activation delay of the vibration mode of operation of the vibrating agitator 2114 such that the vibration mode of operation at least partially transpires within a region of the gastrointestinal tract in which the flowable ingestible medicament is absorbable by the body of the user, and at which it is desirable for the flowable ingestible medicament to be delivered into the gastrointestinal tract.

In some embodiments, control element 2116 is adapted to control a timing or activation delay of the vibration mode of operation of the vibrating agitator 2114 such that the vibration mode of operation at least partially transpires within an estimated absorption time period of the flowable ingestible medicament 2102 within the gastrointestinal tract of the user.

In some embodiments, control element 2116 is adapted to control a timing or activation delay of the vibration mode of operation of the vibrating agitator 2114 such that the vibration mode of operation at least partially transpires within an actual absorption time period of the flowable ingestible medicament 2102 within the gastrointestinal tract of the user.

In the vibrating mode of operation, intermittently activated vibrating agitator 2114 is adapted to have a plurality of vibration cycles, where each cycle includes a vibration duration followed by a repose duration. Forces are exerted by the vibrating agitator 2114 on capsule housing 2112 only during the vibration duration, and as such, capsule housing 2112 only exerts forces on an environment thereof during the vibration duration.

In some embodiments, the number of vibration cycles per hour is in the range of to 400, 40 to 400, 60 to 400, 80 to 400, 40 to 380, 60 to 380, 80 to 380, 40 to 360, 60 to 360, 80 to 360, 100 to 360, 100 to 330, 100 to 300, 100 to 280, 100 to 250, 100 to 220, 100 to 200, 120 to 300, 120 to 280, 120 to 250, 120 to 220, 120 to 200, 150 to 300, 150 to 280, 150 to 250, 150 to 220, 150 to 200, 170 to 300, 170 to 250, 170 to 220, or 170 to 200.

In some embodiments, the repose duration is greater than the vibration duration.

In some embodiments, the vibration duration is in the range of 0.1 second to 10 seconds, 1 second to 10 seconds, 1 second to 9 seconds, 2 seconds to 9 seconds, 3 seconds to 9 seconds, 3 seconds to 8 seconds, 3 seconds to 7 seconds, 3 seconds to 6 seconds, 4 seconds to 6 seconds, or 5 seconds to 6 seconds.

In some embodiments, the repose duration is in the range of 1 second to 180 seconds, 3 seconds to 180 seconds, 5 seconds to 180 seconds, 5 seconds to 150 seconds, seconds to 120 seconds, 8 seconds to 100 seconds, 8 seconds to 30 seconds, 10 seconds to 80 seconds, 10 seconds to 70 seconds, 10 seconds to 60 seconds, 10 seconds to 50 seconds, 10 seconds to 40 seconds, 10 seconds to 30 seconds, 10 seconds to 20 seconds, or 15 seconds to 20 seconds.

In some embodiments, the total duration of one vibration cycle is in the range of 1.1 seconds to 200 seconds, 5 seconds to 200 seconds, 10 seconds to 200 seconds, 10 seconds to 150 seconds, 10 seconds to 100 seconds, 10 seconds to 80 seconds, 10 seconds to 50 seconds, 10 seconds to 40 seconds, 10 seconds to 30 seconds, 15 seconds to 50 seconds, 15 seconds to 40 seconds, 15 seconds to 30 seconds, or 15 seconds to 25 seconds.

In some embodiments, the cumulative duration of the vibrating mode of operation, or the cumulative duration during which vibration cycles are occurring, is in the range of 1 hour to 12 hours, 2 hours to 10 hours, 2 hours to 8 hours, 2 hours to 6 hours, 2 hours to 4 hours, or 2 hours to 3 hours. It will be appreciated that the cumulative duration of vibration cycles may be dependent on properties of power source 2118.

It will be appreciated by persons skilled in the art that the vibration mode of operation may be intermittent, or interrupted, such that vibrating agitator 2114 is operative in the vibration mode for a first duration, for example 30 minutes, then does have any vibration cycles for a second duration, for example 1 hour, and then is operative in the vibration mode and has vibration cycles for a third duration, for example two hours. The cumulative duration relates to the sum of all durations during which vibrating agitator 2114 was operative in the vibration mode and included vibration cycles, including the vibration duration and the repose duration of the vibration cycle.

In some embodiments, vibrating agitator 2114 is configured to exert forces on the capsule housing 2112, such that a net force exerted by the capsule housing 2112 on the environment thereof is in the range of 50 grams force (gf) to 600 gf, 50 gf to 550 gf, 100 gf to 550 gf, 100 gf to 500 gf, 150 gf to 500 gf, 200 gf to 500 gf, or 200 gf to 450 gf.

In some embodiments, vibrating agitator 2114 is configured to exert said forces on capsule housing 2112 to attain a capsule housing 2112 vibrational frequency within a range of 10 Hz to 650 Hz, 15 Hz to 600 Hz, 20 Hz to 550 Hz, 30 Hz to 550 Hz, 50 Hz to 500 Hz, 70 Hz to 500 Hz, 100 Hz to 500 Hz, 130 Hz to 500 Hz, or 150 Hz to 500 Hz.

It will be appreciated that the exact specifications of the capsule, such as the specific frequency and force ranges applicable to a specific capsule, are dependent on the specifications of the power source 2118 and of the vibrating agitator 2114.

It will be further appreciated that a specific capsule may be controlled by the control element 2116 such that different vibrational frequencies may be attained and/or different net forces may be exerted, by the capsule in different vibration cycles of the capsule. Due to the natural distinction between users, use of multiple different parameters in different vibration cycles of a single capsule would allow the capsule to successfully treat multiple users, even if the personal optimal treatment for those users is not the same, as there is a higher chance that in at least some of the vibration cycles the activation parameters of the capsule would reach, or be close to, the optimal parameters for each specific user.

Control element 2116 is adapted to control the operation of intermittently activated vibrating agitator 2114. Such control may include control of any one or more of the force applied by the vibrating agitator 2114, the vibrational frequency reached, the times in which vibrating agitator 2114 operates in the vibration mode of operation, the vibration duration of each vibration cycle, the repose duration of each vibration cycle, the vibration cycle duration, and cumulative vibration duration of the vibrating agitators.

In some embodiments, control element 2116 is adapted to receive information relating to the desired vibration protocol from control unit 2140, prior to ingestion of device 2100 and capsule 2110 or to activation of the capsule, or during the device's and capsule's traversal of the user's GI tract. For example, the information may be remotely transmitted from control unit 2140 to control element 2116, for example using a short range wireless communication method. In some embodiments, the information is transmitted as a list of vibration parameters for effecting the vibration protocol. In some embodiments, the information is transmitted as executable code for effecting the first vibration protocol.

In some embodiments, the information includes a desired number of vibration cycles, a desired vibration duration in each vibration cycle, a desired repose duration in each vibration cycle, a desired cumulative vibration duration, and the like.

In some embodiments, the flowable ingestible medicament is absorbable or at least partially absorbable in the stomach of the user. In some embodiments, the flowable ingestible medicament is absorbable or at least partially absorbable in the small intestine of the user.

In some embodiments, the flowable ingestible medicament has a viscosity in the range of 100 Pa·s to 1000 Pa·s.

In some embodiments, the flowable ingestible medicament is suitable for treatment of one or more symptom or disease, selected from the group consisting of: Parkinsonism; Parkinson's Disease; progressive supranuclear palsy; corticobasal degeneration; multiple system atrophy; Parkinson-plus syndromes (also known as disorders of multiple system degeneration); any neurodegenerative disease in which the subject exhibits at least one (and typically at least two or three) of the classical features of Parkinson's disease: tremor, postural instability, and akinesia or bradykesia; any neurodegenerative disease in which the subject positively responds to a dopaminergic treatment; any neurodegenerative disease in which the particular subject positively responds to an anticholinergic treatment; Constipation; Crohn's disease; Gastroparesis; irritable bowel syndrome (IBS); diarrhea or loose bowel movements; colitis; Hirschsprung's disease; Dyspepsia; and dysphagia.

In some embodiments, the flowable ingestible medicament comprises or includes an ingestible medicament selected from the group consisting of: levodopa; at least one dopaminergic agent; at least one catecholamine precursor; a dopamine precursor; at least one dopamine precursor agent; (L)-3,4-dihydroxyphenylalanine; N-methyl-N-(2-propynyl)-2-methyl-1-phenylethyl-2-amine; tyrosine hydroxylase; apomorphine; at least one anticholinergic agent; at least one agent selected to antagonize at least one cholinergic receptor; benzhexol; orphenadrine; at least one selective allosteric potentiator of metabotropic glutamate receptor 4 (mGluR4); N-phenyl-7-(hydroxylimino)cyclopropa[b]chromen-1a-carboxamide; at least one osmotic agent; magnesium citrate; magnesium hydroxide; polyethylene glycol; sodium phosphate;

MiraLAX®; at least one contraction stimulating agent; bisacodyl; senna; Correctol; Ducodyl; Dulcolax; Senexon; Senokot; at least one stool softening agent; docusate sodium; Colace; Linaclotide; Lactulose; lubiprostone; plecanatide; prucaltride; loperamide; and bismuth subsalicylate.

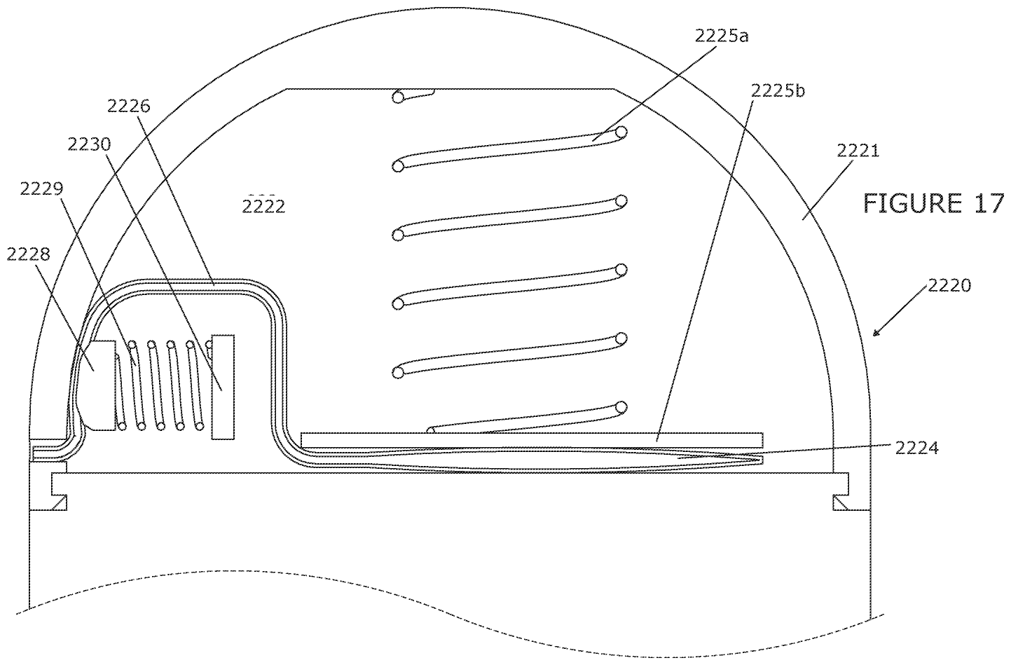

Reference is now made to FIG. 14, which is a planar sectional illustration of a device 2200 for delivering a flowable ingestible medicament 2202 into the gastrointestinal tract of a user according to another embodiment of the present invention, the device including a medicament reservoir 2224 and a valve, to FIG. 15, which is a partial perspective sectional illustration of device 2200, to FIGS. 16A and 16B, which are partial planar sectional illustrations of device 2200, where medicament reservoir 2224 is full, and the valve is in closed and open operative orientations, respectively, and to FIG. 17, which is a partial planar sectional illustrations of device 2200, where medicament reservoir 2124 is empty, and the valve is in a closed operative orientation.

As seen, device 2200, which is arranged along a longitudinal axis, includes a vibrating ingestible capsule 2210 including a housing 2212, substantially as described hereinabove with respect to FIG. 13. It will be appreciated that capsule 2210 includes at least a vibrating agitator, a control element, and a power source, as described hereinabove with respect to FIG. 13, even though these components are not explicitly shown in FIGS. 14A to 16B.

A medicament delivery compartment 2220, here illustrated as having the shape of a convex dome is formed by a medicament compartment housing 2221 which defines a hollow 2222. A portal 2223 is formed in medicament compartment housing 2221. Medicament reservoir 2224 is disposed within hollow 2222, and is biased toward capsule 2210 by a reservoir biasing mechanism. The reservoir biasing mechanism includes a spring 2225a anchored at one end thereof to medicament compartment housing 2221 and terminating, at an opposing end, in a biasing plate 2225b which engages an exterior surface of medicament reservoir 2224 and applies pressure thereto. The medicament reservoir 2224 is flexible and collapsible, and may be formed of any suitable material such as silicone rubber, natural rubber, polyethylene, and PVC.

A conduit 2226, which may be a flexible and/or resilient conduit, extends from medicament reservoir 2224 to portal 2223, and terminates with portal 2223, such that fluid can flow from medicament reservoir 2224, via conduit 2226 and portal 2223, out of the device 2200 and into an environment surrounding the device. In some embodiments, the end of conduit 2226 disposed within portal 2223 also seals the portal, so as to prevent material from the environment entering device 2200. In other embodiments, the end of conduit 2226 may be surrounded by a seal sealing the portal.

A valve 2227 disposed within hollow 2222 includes a weight 2228 attached to a compression spring 2229, which functions as a valve biasing mechanism. The spring 2229 is anchored, at an end distal to weight 2228, to a rigid anchoring shelf 2230 extending from medicament compartment housing 2221 or from housing 2212 of vibrating ingestible capsule 2210.

Biasing spring 2229 and weight 2228 are constructed such that, when vibrating ingestible capsule 2210 is in an inoperative mode, or is in an operative mode but not vibrating, biasing spring 2229 biases weight 2228 against conduit 2226, thus pinching the conduit closed, as illustrated clearly in FIG. 16A. This is the closed operative orientation of valve 2227. When valve 2227 is in the closed operative orientation, no fluid can flow through conduit 2226, and pressure applied by the reservoir biasing mechanism to medicament reservoir 2224 is at an equilibrium with forces resisting such pressure by the content of the medicament reservoir and conduit.

However, when vibrating ingestible capsule 2210 is in the vibrating mode of operation, vibration of the vibrating ingestible capsule 2210 is applied also to biasing spring 2229, via anchoring shelf 2230. As discussed hereinabove, the vibrations of the capsule 2210 are periodic, and cause the spring to periodically contract and extend. Contraction of the spring 2229 results in weight 2228 being withdrawn, or moved away, from conduit 2226, thus enabling the conduit 2226 to recover its nominal diameter and fluid to flow through the conduit 2226, as illustrated in FIG. 16B. This is the open operative orientation of valve 2227. When valve 2227 is in the open operative orientation, pressure applied by the reservoir biasing mechanism to medicament reservoir 2224 causes medicament 2202 to flow from reservoir 2224, via conduit 2226 and portal 2223, into the environment surrounding device 2200.

In use, when capsule 2210 is in the vibrating mode of operation, valve 2227 periodically transitions between the closed operative orientation and the open operative orientation, and vice versa. During such vibration times, and when the valve is in the open operative orientation, flowable ingestible medicament 2202 is delivered from reservoir 2224 to the environment surrounding device 2200. Because of the periodic opening and closing of valve 2227, such delivery occurs in bursts, or quanta, until the medicament reservoir is empty and all the flowable medicament has been delivered, as illustrated in FIG. 17.

The volume of medicament delivered in each such burst, is dependent on the pressure applied by the reservoir biasing mechanism, the recovery time of conduit 2226, the diameter of the conduit, and the duration that valve 2227 is in the open operative orientation. The duration that valve 2227 is in the open operative orientation is based on the frequency of vibrations exerted by vibrating ingestible capsule 2210, as well as on the mass of weight 2228, the length and spring constant of biasing spring 2229, and the rigidity of anchoring shelf 2230. In some embodiments, the valve 2227 functions as a gear reducer, such that the frequency at which the valve transitions between the open and closed configurations is lower than the frequency of vibration of the capsule 2210.

Medicament compartment housing 2221 of medicament delivery compartment 2220 is attached to housing 2212 of vibrating ingestible capsule 2210. In the illustrated embodiment, housing 2212 of vibrating ingestible capsule 2210 includes a first attachment mechanism in the form of a circumferential slot 2250 and a circumferential protrusion 2252 disposed adjacent a longitudinal end 2254 of capsule housing 2212. Medicament compartment housing 2221 includes a second, corresponding attachment mechanism in the form of a circumferential slot 2260 and a circumferential protrusion 2262 disposed adjacent an end 2264 of medicament compartment housing 2221. Circumferential slot 2260 corresponds in dimensions to circumferential protrusion 2252 of capsule 2210, and circumferential protrusion 2262 corresponds in dimensions to circumferential slot 2250 of capsule 2210.

In the illustrated embodiment, medicament compartment housing 2220 is fixedly attached to vibrating ingestible capsule 2210 by snap fit engagement of slot 2260 with protrusion 2252 and snap fit engagement of protrusion 2262 with slot 2250. However, any type of attachment between medicament compartment housing 2221 and vibrating ingestible capsule 2210 is considered within the scope of the present invention, including threaded engagement, engagement by soldering, engagement by adhesive, and the like.

Reference is now made to FIG. 18, which is a schematic diagram of a device 2300 for delivering a flowable ingestible medicament 2302 into the gastrointestinal tract of a user according to yet another embodiment of the present invention

As seen, device 2300, which is arranged along a longitudinal axis, includes a vibrating ingestible capsule 2310 including a housing 2312, formed of a first housing portion and a second housing portion which define a single hollow, and a vibrating agitator 2314, substantially as described hereinabove with respect to FIG. 13. It will be appreciated that capsule 2310 also includes a control element, and a power source, as described hereinabove with respect to FIG. 13, even though these components are not explicitly shown in FIG. 18.

Vibrating ingestible capsule 2310 also functions as a medicament delivery compartment, such that a portal 2323 is formed in the second housing portion of housing 2312. A medicament reservoir 2324 is disposed within a hollow of capsule 2310, and is biased toward a shelf 2320 extending radially inwardly from housing 2312 by a reservoir biasing mechanism. The reservoir biasing mechanism includes a spring 2325a anchored at one end thereof to a longitudinal end of housing 2312 and terminating, at an opposing end, in a biasing plate 2325b which engages an exterior surface of medicament reservoir 2324 and applies pressure thereto. The medicament reservoir 2324 is flexible and collapsible, and may be formed of any suitable material such as silicone rubber, natural rubber, polyethylene, and PVC.

A conduit 2326, which may be a flexible and/or resilient conduit, extends from medicament reservoir 2324 to portal 2323, and terminates with portal 2323, such that fluid can flow from medicament reservoir 2324, via conduit 2326 and portal 2323, out of the device 2300 and into an environment surrounding the device. In some embodiments, the end of conduit 2326 disposed within portal 2323 also seals the portal, so as to prevent material from the environment entering device 2300. In other embodiments, the end of conduit 2326 may be surrounded by a seal sealing the portal.

Vibrating agitator 2314 is attached to a biasing spring 2329, which is anchored to housing 2312. Vibrating agitator 2312 and compression spring 2329 form a valve, which functions in the manner described above with respect to valve 2227 of FIGS. 14 to 17, which functions as a valve biasing mechanism. The spring 2229 is anchored, at an end distal to weight 2228, to a rigid anchoring shelf 2230 extending from medicament compartment housing 2221 or from housing 2212 of vibrating ingestible capsule 2210.

As such, in a closed operative orientation of the valve, biasing spring 2329 biases vibrating agitator 2314 against conduit 2326, thus pinching the conduit closed. In this closed operative orientation, no fluid can flow through conduit 2326, and pressure applied by the reservoir biasing mechanism to medicament reservoir 2324 is at an equilibrium with forces resisting such pressure by the content of the medicament reservoir and conduit.

However, when vibrating agitator 2314 is in the vibrating mode of operation, biasing spring 2329 periodically contracts and extends, resulting in vibrating agitator 2314 being periodically withdrawn, or moved away, from conduit 2326, thus transitioning the valve to an open operative orientation, enabling conduit 2326 to recover its nominal diameter and allowing fluid to flow through conduit 2326. When the valve is in the open operative orientation, pressure applied by the reservoir biasing mechanism to medicament reservoir 2324 causes medicament 2302 to flow from reservoir 2324, via conduit 2326 and portal 2323, into the environment surrounding device 2300.

The delivery of the flowable medicament into the environment is as described hereinabove with respect to FIGS. 14 to 17.

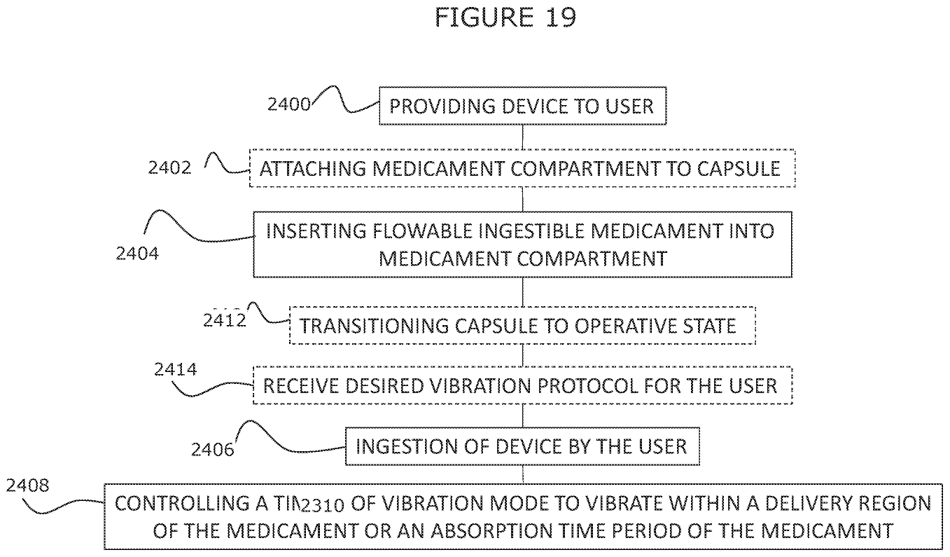

Reference is now additionally made to FIG. 19, which is a schematic flowchart of a method for delivering a flowable ingestible medicament into the gastrointestinal tract of user according to the present invention. The method may be based on the use of a device including a vibrating ingestible capsule and a flowable ingestible medicament, as described hereinabove with reference to FIGS. 13 to 18.

As seen at step 2400, a device, such as device 2100, 2200, or 2300 described hereinabove, including a vibrating ingestible capsule and a medicament delivery compartment, is provided to a user.

In some embodiments, at step 2402, the vibrating ingestible capsule and the medicament delivery compartment are attached to each other. In some embodiments, step 2402 may take place in a factory, prior to providing the device to the user at step 2400. In other embodiments, the device may be provided to the user as two separate pieces, namely the ingestible vibrating capsule and the medicament delivery compartment, and the user carries out step 2402 following receipt of the device at step 2400. In some embodiments, in which the device is constructed as a unitary structure, for example as illustrated in FIG. 18, step 2402 may be obviated.

In some embodiments, the attaching at step 2402 includes fixedly attaching the medicament delivery compartment to the vibrating ingestible capsule.

In some embodiments, the attaching at step 2402 includes removably attaching the medicament delivery compartment to the vibrating ingestible capsule.

In some embodiments, the attaching at step 2402 includes attaching the medicament delivery compartment to the vibrating ingestible capsule by one or more of snap fit engagement, threaded engagement, adhering, soldering, or any other suitable mechanism of attachment.

In some embodiments, the attaching at step 2402 includes mutually attaching a first attachment mechanism on the vibrating ingestible capsule with a corresponding attachment mechanism on the medicament delivery compartment, for example as described with respect to FIGS. 14 to 17.

In some embodiments, the device is provided to the user having the flowable ingestible medicament disposed within the medicament reservoir and within the medicament delivery compartment.

In other embodiments, at step 2404, the flowable ingestible medicament is inserted into medicament delivery compartment. Step 2404 may include filling of the medicament reservoir with the flowable ingestible medicament and/or inserting the medicament reservoir into the medicament delivery compartment or into the device.

In some embodiments, step 2404 takes place prior to attaching the ingestible vibrating capsule with the medicament delivery compartment, either in a factory or by the user. The flowable ingestible medicament inserted at step 2404 may be any suitable type of flowable ingestible medicament, as described in detail hereinabove.

Regardless of when the medicament reservoir is inserted into the device, the medicament reservoir is placed under pressure within the device, as explained hereinabove.

At step 2406, the device, including the vibrating ingestible capsule, the medicament delivery compartment, and the flowable ingestible medicament, is ingested by the user, and begins to travel through the gastrointestinal tract of the user.

At step 2408, which occurs following the user ingesting the device at step 2406, the vibrating ingestible capsule is controlled such that the vibration mode of operation (e.g., when the vibration mode is initiated, a duration of the vibration mode, etc.) at least partially transpire within an area of the gastrointestinal tract at which the flowable ingestible medicament should be delivered, or within an absorption time period of the flowable ingestible medicament within the gastrointestinal tract of the user.

The absorption time period may be an estimated absorption time period, as defined herein, and/or an actual absorption time period as defined herein.

In some embodiment, step 2408 may include controlling a timing of the vibration mode of operation such that the vibration mode at least partially transpires when the capsule is in a region of the gastrointestinal tract in which the flowable ingestible medicament is typically absorbed into the bloodstream. The region of the gastrointestinal tract may include one or more of the stomach of the user, the duodenum of the user, the small intestine of the user, the large intestine of the user, or the colon of the user.

For example, when the flowable ingestible medicament is levadopa, which is typically absorbed into the bloodstream through the stomach walls and/or the small intestine walls, the vibration mode at least partially transpires within a time period in which the device traverses, or is expected to traverse, the stomach and small intestine.

In some embodiments, step 2408 includes setting at least one vibration parameter of the vibrating ingestible capsule of the device so as to promote delivery of flowable ingestible medicament into the gastrointestinal tract of the user or absorption of the ingestible medicament into the bloodstream of the user. In some such embodiments, the at least one vibration parameter set at step 2408 includes at least one of a vibration frequency, a cumulative vibration duration, a number of vibration cycles per time unit, a duration of a vibration duration within a vibration cycle, a duration of a repose duration within a vibration cycle, a total duration of a single vibration cycle, and a net force exerted by said housing on said environment.

In some embodiments, the controlling at step 2408 includes controlling the vibrating agitator such that the vibrating mode of operation includes a plurality of cycles, each of the cycles including a vibration duration followed by a repose duration, wherein the housing exerts the vibrations during the vibration duration.

In some embodiments, the repose duration is greater than the vibration duration.

In some embodiments, the vibration duration is in the range of 0.1 second to 10 seconds, 1 second to 10 seconds, 1 second to 9 seconds, 2 seconds to 9 seconds, 3 seconds to 9 seconds, 3 seconds to 8 seconds, 3 seconds to 7 seconds, 3 seconds to 6 seconds, 4 seconds to 6 seconds, or 5 seconds to 6 seconds.

In some embodiments, the repose duration is in the range of 1 second to 180 seconds, 3 seconds to 180 seconds, 5 seconds to 180 seconds, 5 seconds to 150 seconds, seconds to 120 seconds, 8 seconds to 100 seconds, 8 seconds to 30 seconds, 10 seconds to 80 seconds, 10 seconds to 70 seconds, 10 seconds to 60 seconds, 10 seconds to 50 seconds, 10 seconds to 40 seconds, 10 seconds to 30 seconds, 10 seconds to 20 seconds, or 15 seconds to 20 seconds.

In some embodiments, a duration of each of the plurality of cycles is in the range of 1.1 seconds to 200 seconds, 5 seconds to 200 seconds, 10 seconds to 200 seconds, 10 seconds to 150 seconds, 10 seconds to 100 seconds, 10 seconds to 80 seconds, 10 seconds to 50 seconds, 10 seconds to 40 seconds, 10 seconds to 30 seconds, 15 seconds to 50 seconds, 15 seconds to 40 seconds, 15 seconds to 30 seconds, or 15 seconds to 25 seconds.

In some embodiments, the controlling at step 2408 includes controlling the vibrating agitator such that a cumulative duration of the vibrating mode of operation is in the range of 1 hour to 12 hours, 2 hours to 10 hours, 2 hours to 8 hours, 2 hours to 6 hours, 2 hours to 4 hours, or 2 hours to 3 hours.

In some embodiments, the controlling at step 2408 includes controlling the vibrating agitator to exert forces on the housing of the vibrating ingestible capsule, such that a net force exerted by the housing on the environment thereof is in the range of 50 grams force (gf) to 600 gf, 50 gf to 550 gf, 100 gf to 550 gf, 100 gf to 500 gf, 150 gf to 500 gf, 200 gf to 500 gf, or 200 gf to 450 gf.

In some embodiments, the controlling at step 2408 includes controlling the vibrating agitator to exert the forces on the housing to attain a housing vibrational frequency within a range of 10 Hz to 650 Hz, 15 Hz to 600 Hz, 20 Hz to 550 Hz, 30 Hz to 550 Hz, 50 Hz to 500 Hz, 70 Hz to 500 Hz, 100 Hz to 500 Hz, 130 Hz to 500 Hz, or 150 Hz to 500 Hz.

In some embodiments, and as described in further detail herein, the method may include a further step 2412 of transitioning the capsule (from an inoperative state) to an operative state.

The capsule may be pre-programmed with a vibration protocol. This protocol may include, by way of example, a particular or pre-determined activation time following ingestion, in which the capsule is transitioned from an inoperative state to an operative state. In such embodiments, the step 2412 may be omitted from the method.

Alternatively or additionally, the capsule may receive an activation input in an active fashion (e.g., from an external controller via RF) or in a passive fashion (e.g., a signal from a sensor to the on-board controller), as described in detail hereinabove. It will be appreciated that step 2412, in which the vibrating ingestible capsule is transitioned from the inoperative state to the operative state, may be performed prior to ingestion of the device by the user in step 2406, or following such ingestion, for example in the case of external control via RF.

Substantially as described hereinabove, step 2412 may be carried out, and the vibrating ingestible capsule may be activated, prior to the user ingesting the capsule at step 2406, for example by a signal from the control unit or by the user carrying out an activation motion. In other embodiments, the activation input, and the transitioning of the capsule from being inoperative to being operative, occurs at the time of ingestion or immediately thereafter, for example by sensors sensing a change in the environment of the capsule due to its ingestion, as described at length hereinabove. In yet other embodiments, the transitioning of the capsule at step 2412 may include the capsule receiving an activation input which is provided remotely when the capsule is already in the body of the user, for example by remote communication from control module 2140. In some embodiments, a control element of the vibrating ingestible capsule may optionally receive a desired vibration protocol for the user, at an optional step 2414. In some embodiments, the programming of the desired vibration protocol at step 2414 occurs at the time of manufacturing of the vibrating ingestible capsule or of the device, for example by pre-programming the protocol into the control element. In other embodiments, providing the desired vibration protocol for the user at step 2414 may be effected by a control unit, such as control unit 2140 of FIG. 13, as described in detail hereinabove with respect to FIG. 13.

In some embodiments, following expelling of the capsule from the subject's body, the capsule may be detected by a toilet bowl mounted sensor, thereby to confirm that the capsule has been expelled from the subject's body.

It will be appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1-6. (canceled)

7. Use of a vibrating ingestible capsule for promoting absorption of an ingested medicament into the blood stream, the vibrating ingestible capsule comprising:
a housing;
a vibrating agitation mechanism adapted such that, in a vibration mode of operation, said housing exerts vibrations on an environment surrounding said vibrating gastrointestinal capsule;
a power supply disposed within said housing and adapted to power said vibrating agitation mechanism; and
a control mechanism adapted to activate said vibrating agitation mechanism to operative in said vibration mode of operation, said control mechanism adapted to control a timing or activation delay of said vibration mode of operation such that a first occurrence of said vibration mode of operation at least partially transpires within at least one of an estimated absorption time period and an actual absorption time period of said ingested medicament within said gastrointestinal tract of said subject.

8-14. (canceled)

15. A method of using a vibrating ingestible capsule in coordination with an ingestible medicament, the method comprising:
(a) providing the vibrating ingestible capsule, the capsule including:
a housing;
a vibrating agitation mechanism adapted such that, in a vibration mode of operation, said housing exerts vibrations on an environment surrounding the vibrating ingestible capsule;
a power supply disposed within said housing and adapted to power said vibrating agitation mechanism; and
a control mechanism adapted to activate said vibrating agitation mechanism to operate in said vibration mode of operation;
(b) ingesting the ingestible medicament;
(c) ingesting the vibrating ingestible capsule; and
(d) controlling at least one of a time of said ingesting of the vibrating ingestible capsule and a timing or activation delay of said vibration mode of operation, such that a first occurrence of said vibration mode of operation at least partially transpires within at least one of an estimated absorption time period and an actual absorption time period of the ingestible medicament within the gastrointestinal tract of the subject.

16. The method of claim 15, wherein said controlling is effected such that said vibration mode of operation at least partially transpires during said actual absorption time.

17. The method of claim 15, wherein said controlling is effected such that said vibration mode of operation at least partially transpires during said estimated absorption time.

18. The method of claim 17, wherein said estimated absorption time is within a range of 0.5 to 5 hours from said ingesting of said ingestible medicament.

19. The method of claim 15, wherein said ingesting of said vibrating ingestible capsule occurs after said ingesting of said ingestible medicament, and transpires within 4 hours of said ingesting of the ingestible medicament.

20. The method of claim 15, wherein said ingesting of said vibrating ingestible capsule is simultaneous with said ingesting of the ingestible medicament.

21. The method of claim 15, further comprising timing said vibration mode of operation to at least partially transpire within 5 hours of said ingesting of the ingestible medicament.

22. The method of claim 15, wherein said first occurrence of said vibration mode of operation at least partially transpiring within at least one of an estimated absorption time period and an actual absorption time period of the ingestible medicament within the gastrointestinal tract of the subject effects an increased absorption of the ingestible medicament, so as to improve a therapeutic efficacy of the medicament.

23. The method of claim 15, wherein said first occurrence of said vibration mode of operation at least partially transpiring within at least one of an estimated absorption time period and an actual absorption time period of the ingestible medicament within the gastrointestinal tract of the subject effects an increased absorption of the ingestible medicament, so as to enable the use of a lower dosage of said medicament than a dosage used without use of said vibrating ingestible capsule, without impairing or diminishing therapeutic efficacy.

24. The method of claim 15, wherein a second occurrence of said vibration mode of operation is effected within the gastrointestinal tract so as to achieve at least one of:
stimulate the enteric nervous system of the subject;
induce at least one peristaltic wave in a wall of the gastrointestinal tract; and
effect increasing peristalsis in a wall of the gastrointestinal tract.
